SEQUENCE LISTING (36 sequences)

SEQ ID NO: 1 - DNA, 1299 bp, Escherichia coli
atggctgaaagtactgtaacggcagacagcaaactgacaagtagtgatactcgtcgccgcatttgggcgattgtgggggcctcttcaggtaatctggtcgagtggttcgatttctatgtctactcgttctgttcactctactttgcccacatcttcttcccttccgggaacacgacgactcaactactacaaacagcaggtgtttttgctgcgggattcctgatgcgcccaataggcggttggctatttggccgcatagccgataaacatggtcgcaaaaaatcgatgctgttatcggtgtgtatgatgtgtttcggatcgctggttatcgcctgcctcccaggttatgaaactataggtacgtgggctccggcattattgcttctcgctcgtttatttcagggattatctgttggcggagaatatggcaccagcgccacctatatgagtgaagttgccgttgaagggcgcaaaggtttttacgcatcatttcagtatgtgacgttgatcggcggacaactgctagccctactggttgtcgtggttttacaacacaccatggaagacgctgcactcagagagtggggatggcgtattcctttcgcgttaggagctgtgttagctgttgtggcgttgtggttacgtcgtcagttagatgaaacttcgcaacaagaaacgcgcgctttaaaagaagctggatctctgaaaggattatggcgcaatcgccgtgcattcatcatggttctcggttttaccgctgcgggctccctttgtttctataccttcactacttatatgcagaagtatctggtaaatactgcgggaatgcatgccaacgtggcgagtggcattatgactgccgcattgtttgtattcatgcttattcaaccactcattggcgcgctgtcggataagattggtcgccgtacctcaatgttatgtttcggttcgctggcagccatttttaccgttcctattctctcagcattgcaaaacgtttcctcgccttatgccgcttttggtctggtgatgtgtgccctgctgatagtgagtttttatacatcaatcagtggaatactgaaggctgagatgttcccggcacaggttcgcgcattaggcgttggtctgtcatatgcggtcgctaatgctatatttggtggttcggcggagtacgtagcgttgtcgctgaaatcaataggaatggaaacagccttcttctggtatgtgaccttgatggccgtggtggcgtttctggtttctttgatgctacatcgcaaagggaaggggatgcgtctttag

SEQ ID NO: 2 - DNA, 1686 bp, Escherichia coli
gtgaatataaacgtcgccgaattgttaaatgggaattacattctgttattatttgtggtcctcgcgcttgggctatgtctcggaaagttacgacttggttcgatccaactgggtaattccattggcgttttagtcgtatcgctgttattaggccaacaacatttcagcattaacaccgatgcgcttaatcttggctttatgctgtttattttctgcgtcggggtcgaagccggaccgaactttttttccattttttttcgcgatgggaaaaattacctaatgttagcactggtgatggttggcagtgcgctggtgatcgccttagggttaggtaagctgtttggctgggatattggcctgacggccggtatgttagcaggctctatgacgtcgacaccggttctggtcggtgctggcgatacactgcgtcattccggcatggaaagcaggcagctctcactggcactggataatctgagcctcgggtatgccttaacctatttaatcggtctggtgagtttgattgttggtgcgcgttacttgccgaaattgcagcatcaggacttacagaccagcgcccagcaaatcgcccgcgaacgtggcctggacactgatgccaaccgtaaggtttatttaccggtgatccgcgcctatcgcgtcggcccggaactggtggcctggaccgacggcaaaaatctgcgtgaactgggtatttatcgacaaaccggctgctacattgaacgtattcgacgtaacgggattctggcaaatccagacggtgatgccgtgctacaaatgggcgatgaaatagcgttggtaggctatcccgacgcccatgcccgactcgatcccagcttccgtaacggtaaagaagttttcgatcgtgaccttctcgacatgcgtatcgtcactgaagaagtggtcgttaaaaaccataacgctgtaggtaaacgtctcgcacaactgaagttgaccgatcacggttgcttccttaaccgcgtcattcgtagccagattgagatgccgatagatgacaacgtcgtgcttaacaaaggtgacgttttacaagtcagcggcgatgcccgccgcgtaaaaaccatcgccgatcgcatcggctttatctcgattcacagccaggtcactgacctgctggcattctgcgccttctttgttattgggctgatgatcgggatgatcaccttccagttcagcacattcagtttcggcatggggaacgctgccgggttgttattcgccggaattatgctgggctttatgcgtgctaaccacccgaccttcggttacattccgcagggtgcattaagcatggtgaaagagttcggcttgatggtgtttatggcaggcgttggtctgagcgccggtagcggtattaataacggcctgggcgcgattggcggtcagatgttgattgccggattgattgtcagtctggtgcccgtggttatctgtttcttgttcggtgcttatgtattgcgaatgaaccgcgcgctgttgttcggcgcaatgatgggcgcacgtacctgcgcgccggcaatggagatcatcagtgatacagctcgcagtaacatcccggcgctgggctatgcgggcacctatgcaatcgccaacgtcctgctgacgctggcagggacaatcatcgtcatggtatggccaggattaggataa

SEQ ID NO: 3 - DNA, 1203 bp, Escherichia coli
atggcaattaaattagaaattaaaaatctttataaaatatttggcgagcatccacagcgagcgttcaaatatatcgaacaaggactttcaaaagaacaaattctggaaaaaactgggctatcgcttggcgtaaaagacgccagtctggccattgaagaaggcgagatatttgtcatcatgggattatccggctcgggtaaatccacaatggtacgccttctcaatcgcctgattgaacccacccgcgggcaagtgctgattgatggtgtggatattgccaaaatatccgacgccgaactccgtgaggtgcgcagaaaaaagattgcgatggtcttccagtcctttgccttaatgccgcatatgaccgtgctggacaatactgcgttcggtatggaattggccggaattaatgccgaagaacgccgggaaaaagcccttgatgcactgcgtcaggtcgggctggaaaattatgcccacagctacccggatgaactctctggcgggatgcgtcaacgtgtgggattagcccgcgcgttagcgattaatccggatatattattaatggacgaagccttctcggcgctcgatccattaattcgcaccgagatgcaggatgagctggtaaaattacaggcgaaacatcagcgcaccattgtctttatttcccacgatcttgatgaagccatgcgtattggcgaccgaattgccattatgcaaaatggtgaagtggtacaggtcggcacaccggatgaaattctcaataatccggcgaatgattatgtccgtaccttcttccgtggcgttgatattagtcaggtattcagtgcgaaagatattgcccgccggacaccgaatggcttaattcgtaaaacccctggcttcggcccacgttcggcactgaaattattgcaggatgaagatcgcgaatatggctacgttatcgaacgcggtaataagtttgtcggcgcagtctccatcgattcgcttaaaaccgcgttaacgcagcagcaaggtcttgatgcggcgctgattgatgcgccgttagcagtcgatgcacaaacgcctcttagcgagttgctctctcatgtcggacaggcaccctgtgcggtgcccgtggtcgacgaggaccaacagtatgtcggcatcatttcgaaaggaatgctgctgcgcgctttagatcgtgagggggtaaataatggctga

SEQ ID NO: 4 - DNA, 1065 bp, Escherichia coli
atggctgatcaaaataatccgtgggataccacgccagcggcggacagtgccgcgcaatccgcagacgcctggggtacaccgacgactgcaccgactgacggcggtggtgctgactggctgaccagtacgcctgcgccaaacgtcgagcattttaatattctcgatccgttccataaaacgctgatcccgctcgacagttgggtcactgaagggatcgactgggtcgttacccatttccgtcccgtcttccagggcgtgcgcgttccggttgattatatcctcaacggtttccagcaattgctgctgggtatgcccgcaccggtggcgattatcgttttcgctctcatcgcctggcagatttccggggtcggaatgggtgtggcgacgctggtttcgctgattgccatcggcgcaatcggtgcctggtcgcaggcaatggtgactctggcgctggtgttaaccgccctgctgttctgtatcgtcatcggtttgccgttggggatatggctggcgagaagtccgcgagcggcgaaaattattcgtccactgcttgatgccatgcagaccacgccagcgtttgtttatctggtgccaatcgtcatgctatttggtatcggtaacgtgccgggcgtggtggtgacgatcatctttgctctgccgccgattatccgtctgaccattctggggattaaccaggttccggcggatctgattgaagcctcgcgctcattcggtgccagcccgcgccagatgctgttcaaagttcagttaccgctggcgatgccgaccattatggcgggcgttaaccagacgctgatgctggccctttctatggtggtcatcgcctcgatgattgccgtcggcgggttgggtcagatggtacttcgcggtatcggtcgtctggatatggggcttgccaccgttggcggcgtcgggattgtgatcctcgccattatcctcgatcgtctgacgcaggccgttgggcgcgactcacgcagtcgcggcaaccgtcgctggtacaccactggccctgttggtctgctgacccgcccattcattaagtaa

SEQ ID NO: 5 - DNA, 993 bp, Escherichia coli
atgcgacatagcgtactttttgcgacagcgtttgccacgcttatctctacacaaacttttgctgccgatctgccgggcaaaggcattactgttaatccagttcagagcaccatcactgaagaaaccttccagacgctgctggtcagtcgtgcgctggagaaattaggttataccgtcaacaaacccagcgaagtagattacaacgttggctacacctcgcttgcttccggcgatgcaaccttcaccgccgtgaactggacgccactgcatgacaacatgtacgaagctgccggtggcgataagaaattttatcgtgaaggggtatttgttaacggcgcggcacagggttacctgatcgataagaaaaccgccgaccagtacaaaatcaccaacatcgcacaactgaaagatccgaagatcgccaaactgttcgataccaacggcgacggaaaagcggatttaaccggttgtaaccctggctggggctgcgaaggtgcgatcaaccaccagcttgccgcgtatgaactgaccaacaccgtgacgcataatcaggggaactacgcagcgatgatggccgacaccatcagtcgctacaaagagggcaaaccggtgttttattacacctggacgccgtactgggtgagtaacgaactgaagccgggcaaagatgtcgtctggttgcaggtgccgttctccgcactgccgggcgataaaaacgccgataccaaactgccgaatggtgcgaattatggcttcccggtcagcaccatgcatatcgttgccaacaaagcctgggccgagaaaaacccggcagcagcgaaactgtttgccattatgcagttgccagtggcagatattaacgcccagaacgccattatgcatgacggcaaagcctcagaaggcgatattcagggacacgttgatggttggatcaaagcccaccagcagcagttcgatggctgggtgaatgaggcgctggcagcgcagaagtaa

SEQ ID NO: 6 - DNA, 699 bp, Escherichia coli
atggaaaatcaacctaagttgaatagcagtaaagaagtaatcgcgtttctggccgaacgttttccccactgtttcagtgcggaaggtgaagcgcgtccgctgaaaatcggtatttttcaggatttggtcgatcgtgttgctggggaaatgaacctgagcaaaacgcaattgcgatccgctttacgtctctacacttcgagctggcgttatctttacggtgttaaacccggcgcaacgcgtgtcgatcttgacggcaacccatgcggtgagctggacgagcaacatgtagagcatgctcgcaagcagcttgaagaagcgaaagcgcgtgttcaggcacagcgtgctgaacagcaagcgaaaaaacgcgaagctgccgcaactgctggtgagaaagaagacgcaccgcgccgcgaacgcaagccacgtccgactacgccacgccgcaaagaaggcgctgaacgtaaacctcgtgcgcaaaagccggtagagaaagcgccaaaaacagtaaaagcacctcgcgaagaacagcacaccccggtttctgacatttcagctctgactgtcggacaagccctgaaggtgaaagcgggtcaaaacgcgatggatgccaccgtattagaaatcaccaaagacggcgtccgcgtccagctgaattcgggtatgtctttgattgtgcgcgcagaacacctggtgttctga

SEQ ID NO: 7 - DNA, 210 bp, Escherichia coli
atgtctaagattaaaggtaacgttaagtggtttaatgagtccaaaggattcggtttcattactccggaagacggcagcaaagacgtgttcgtacacttctctgcaatccagactaatggttttaaaactcttgctgaaggtcagcgcgtagagttcgaaatcactaacggtgccaaaggcccttctgctgcaaacgtaatcgctctgtaa

SEQ ID NO: 8 - DNA, 1434 bp, Escherichia coli
atgaaagtaacgctgccagagtttgaacgtgcaggagtgatggtggttggtgatgtgatgctggatcgttactggtacggccccaccagtcgtatctcgccggaagcgccggtgcccgtggttaaagtgaataccatcgaagaacgtccgggcggcgcggctaacgtggcgatgaatatcgcttctctcggtgctaatgcacgcctggtcgggttgacgggcattgacgatgcagcgcgcgcgctgagtaaatctctggccgacgtcaacgtcaaatgcgacttcgtttctgtaccgacgcatccgaccattaccaaattacgggtactttcccgcaaccaacagctgatccgtctggattttgaagaaggtttcgaaggtgttgatccgcagccgctgcacgagcggattaatcaggcgctgagttcgattggcgcgctggtgctttctgactacgccaaaggtgcgctggcaagcgtacagcagatgatccaactggcgcgtaaagcgggtgttccggtgctgattgatccaaaaggtaccgattttgagcgctaccgcggcgctacgctgttaacgccgaatctctcggaatttgaagctgttgtcggtaaatgtaagaccgaagaagagattgttgagcgcggcatgaaactgattgccgattacgaactctcggctctgttagtgacccgttccgaacagggtatgtcgctgctgcaaccgggtaaagcgccgctgcatatgccaacccaagcgcaggaagtgtatgacgttaccggtgcgggcgacacggtgattggcgtcctggcggcaacgctggcagcgggtaattcgctggaagaagcctgcttctttgccaatgcggcggctggcgtggtggtcggcaaactgggaacctccacggtttcgccgatcgagctggaaaatgctgtacgtggacgtgcagatacaggctttggcgtgatgaccgaagaggaactgaagctggccgtagcggcagcgcgtaaacgtggtgaaaaagtggtgatgaccaacggtgtctttgacatcctgcacgccgggcacgtctcttatctggcaaatgcccgcaagctgggtgaccgcttgattgttgccgtcaacagcgatgcctccaccaaacggctgaaaggggattcccgcccggtaaacccactcgaacagcgtatgattgtgctgggcgcactggaagcggtcgactgggtagtgtcgtttgaagaggacacgccgcagcgcttgatcgccgggatcttgccagatctgctggtgaaaggcggcgactataaaccagaagagattgccgggagtaaagaagtctgggccaacggtggcgaagtgttggtgctcaactttgaagacggttgctcgacgaccaacatcatcaagaagatccaacaggataaaaaaggctaa

SEQ ID NO: 9 - DNA, 849 bp, Escherichia coli
atgaaacttattcctcgcagtagtgatatttcacctggcattgatggaatttgcccggggccttttcctccgaacggatttaccgtacttacagatgcagcatatggtaacggagactgtttcggtttgtactggcctattggtcaggagcataaactacctatagtgtgcgaaacgtaccatgacgaatggcgaatcgtacctgccttttccagcattaagaagttcgaagaatggctggaagtgaatgatgatgatccccatgaaaatggcatcagcatcgaagatcaagactttgcagcaaatctcttccgtgttgctcggaaatgtctttcaacgggaaggcttgatgacgcactgccactgttacagcgagcaacagaacaattgcctgaagtcagcgaatattggttagcactggcaatccagtatcgccgatgtaaaaaaacagaagctgcagcacaggctgcgcttaatgcttatcttggcaattgggcatttggtgttcctgacaataaagtcattcatttgctgtcacaggccgcagatgtcccgaatttccaggatgatcctgtgattcagtgcattaaagaacaaggattagatcttagctttggaggaacaaaggaaaataataattaccccttgatgcagatgtgtgttgatacttattttgcgcagagaaaaccgcttcaggcattaaccctgttacataattatgcctggataatgtccagtgaaacgactgcatttcaggaacgctatgattttaatatcgatgaatggcgggcaaaattcaggcagttatgtttagagtattttggcgatagtcgtacacagttcacttaa

SEQ ID NO: 10 - DNA, 264 bp, Escherichia coli
atgggggatgatgtccatcgtttcggtaacctcttcctgatctcaccaagcagcaactcgatacttagtaattattcgccagcagacaagaaaaaattctacgtcgaaacagaacgagcagagagcccaaaacaagccatcatgatgagctataaggagtggggtccggatgggcaaggcatcaacaatatagaaagccatgaacacgcgatgttgacattgctcaaagagcatcgagatatgaccctaccaaccaggaaataa

SEQ ID NO: 11 - DNA, 1041 bp, Escherichia coli
atgaaagttatgcgtaccaccgtcgcaactgttgtcgccgcgaccttatcgatgagtgctttctctgtgtttgcagaagcaagcctgacaggtgcaggtgcaaccttccctgcgccggtgtatgccaaatgggctgacacttaccagaaagaaaccggtaataaagttaactaccagggtatcggttcttccggtggcgtaaaacagattatcgctaataccgttgattttggtgcctctgacgcgccgctgtctgacgaaaaactggctcaggaaggtctgttccagttcccgaccgtgattggcggcgtggtgctggcggttaacattccagggctgaagtctggcgaactggtgctggatggtaaaaccctcggcgacatctacctgggcaaaatcaagaagtgggatgatgaagccatcgccaaactgaatccgggtctgaaactgccttcacaaaacattgctgtagtacgccgcgcagatggctccgggacttccttcgtcttcaccagctacctggcgaaagtgaacgaagagtggaaaaacaacgttggtactggctctaccgtaaaatggccgatcggtctgggcggtaaaggtaacgacggtatcgccgcgttcgttcagcgtctgccgggtgcaattggttatgttgaatatgcttacgcgaagcagaacaacctggcgtacaccaaactgatctccgctgatggtaaaccggttagtccgaccgaagaaaacttcgctaatgcagcaaaaggtgcagactggagcaaaaccttcgctcaggatctgaccaaccagaaaggcgaagatgcatggcctattacctctaccacgttcattctgatccacaaagatcagaagaaaccagaacaaggcacagaagtgctgaaattcttcgactgggcgtacaaaaccggggctaaacaggcgaacgacctggattacgccagcctgccggatagtgtagttgaacaggttcgcgctgcgtggaagaccaatattaaagacagtagcggtaagccgctgtactaa

SEQ ID NO: 12 - DNA, 716 bp, Escherichia coli
atgcgtccagcaggccgtagcaataatcaggtgcgtcccgttaccctgactcgtaactatacaaaacatgcagaaggctcggtgctggtcgaatttggcgataccaaagtgttgtgtaccgcctctattgaagaaggcgtgccgcgcttcctgaaaggtcagggccagggctggatcaccgcagagtacggcatgctgccacgttctacccacacccgtaacgctcgtgaagcggcgaaaggtaagcagggtggacgcacaatggaaatccagcgtctgatcgcccgtgctcttcgcgcggcagtagatttgaaagcgctgggtgagttcaccattacgctggactgcgacgtgcttcaggctgatggtggcacgcgtaccgcgtcgattacgggtgcctgcgtggcgctggtagatgcgctacagaagctggtggaaaacggcaagctgaaaaccaatccgatgaaagggatggtagccgcagtttctgtcggaattgtgaacggcgaagcggtttgcgatctggaatacgttgaagactctgccgcagagaccgacatgaacgtagtgatgaccgaagacgggcgcatcattgaagtgcaggggacggcagaaggcgagccgttcacccatgaagagctactcatcttgttggctctggcccgaggggaatcgaatccattgtagcgacgcagaaggcggcgctggcaaactga

SEQ ID NO: 13 - DNA, 993 bp, Escherichia coli
atgagtcagaatacgctgaaagttcatgatttaaatgaagatgcggaatttgatgagaacggagttgaggtttttgacgaaaaggccttagtagaacaggaacccagtgataacgatttggccgaagaggaactgttatcgcagggagccacacagcgtgtgttggacgcgactcagctttaccttggtgagattggttattcaccactgttaacggccgaagaagaagtttattttgcgcgtcgcgcactgcgtggagatgtcgcctctcgccgccggatgatcgagagtaacttgcgtctggtggtaaaaattgcccgccgttatggcaatcgtggtctggcgttgctggaccttatcgaagagggcaacctggggctgatccgcgcggtagagaagtttgacccggaacgtggtttccgcttctcaacatacgcaacctggtggattcgccagacgattgaacgggcgattatgaaccaaacccgtactattcgtttgccgattcacatcgtaaaggagctgaacgtttacctgcgaaccgcacgtgagttgtcccataagctggaccatgaaccaagtgcggaagagatcgcagagcaactggataagccagttgatgacgtcagccgtatgcttcgtcttaacgagcgcattacctcggtagacaccccgctgggtggtgattccgaaaaagcgttgctggacatcctggccgatgaaaaagagaacggtccggaagataccacgcaagatgacgatatgaagcagagcatcgtcaaatggctgttcgagctgaacgccaaacagcgtgaagtgctggcacgtcgattcggtttgctggggtacgaagcggcaacactggaagatgtaggtcgtgaaattggcctcacccgtgaacgtgttcgccagattcaggttgaaggcctgcgccgtttgcgcgaaatcctgcaaacgcaggggctgaatatcgaagcgctgttccgcgagtaa

SEQ ID NO: 14 - DNA, 639 bp, Echerichia coli
atggctgtcgctgccaacaaacgttcggtaatgacgctgttttccggtcctactgacatctatagccatcaggtccgcattgtgctggctgagaaaggtgtaagtttcgagatcgaacacgtggaaaaggacaatccgcctcaggatctgattgacctcaacccgaatcagagcgttccgaccctggtggatcgtgagctgaccctgtgggaatctcgcatcattatggaatatctggatgagcgtttcccgcatccgccactgatgcctgtttacccggtagctcgcggtgaaagccgtctgtacatgcatcgcatcgaaaaagactggtacacgctgatgaacaccatcatcaacggttcagcttctgaagcagatgccgcacgtaagcaactgcgcgaagaactgctggcgattgcgccggtcttcggtcagaagccgtacttcctgagcgatgagttcagcctggtcgattgctatcttgctccgctgctgtggcgtctgccgcaactgggcatcgagttcagcggcccgggtgcgaaagagctgaaaggctatatgacccgcgtctttgagcgtgactctttccttgcttctttaactgaagcagaacgtgaaatgcgtctgggccggagttaa

SEQ ID NO: 15 - DNA, 618 bp, Escherichia coli
atggcacagctatatttctactattccgcaatgaatgcgggtaagtctacagcattgttgcaatcttcatacaattaccaggaacgcggcatgcgcactgtcgtatatacggcagaaattgatgatcgctttggtgccgggaaagtcagttcgcgtataggtttgtcatcgcctgcaaaattatttaaccaaaattcatcattatttgatgagattcgtgcggaacatgaacagcaggcaattcattgcgtactggttgatgaatgccagtttttaaccagacaacaagtatatgaattatcggaggttgtcgatcaactcgatatacccgtactttgttatggtttacgtaccgattttcgaggtgaattatttattggcagccaatacttactggcatggtccgacaaactggttgaattaaaaaccatctgtttttgtggccgtaaagcaagcatggtgctgcgtcttgatcaagcaggcagaccttataacgaaggtgagcaggtggtaattggtggtaatgaacgatacgtttctgtatgccgtaaacactataaagaggcgttacaagtcgactcattaacggctattcaggaaaggcatcgccacgattaa

SEQ ID NO: 16 - DNA, 2022 bp, Escherichia coli
atgagtaaaccgttcaaactgaattccgcttttaaaccttctggcgatcagccagaggcgattcgacgtctcgaagaggggctggaagatggcctggcgcaccagacgttacttggcgtgactggctcagggaaaaccttcaccattgccaatgtcattgctgaccttcagcgcccaaccatggtacttgcgcccaacaaaacgctggcggcccagctgtatggcgaaatgaaagagttcttcccggaaaacgcggtggaatatttcgtttcctactacgactactatcagccggaagcctatgtaccgagttccgacactttcattgagaaagatgcctcggttaacgaacatattgagcagatgcgtttgtccgccaccaaagcgatgctggagcggcgtgatgtggttgtggtggcgtctgtttccgcgatttatggtctgggcgatcctgatttatatctcaagatgatgctccatctcacggtcggtatgattatcgatcagcgcgcgattctgcgccgactggcggagctgcaatacgctcgtaatgatcaagcattccagcgtggtactttccgcgttcgtggcgaggtgatagatatcttcccggcagaatcggatgacattgcacttcgcgtggaactgtttgacgaggaagtggaacgattgtcgttatttgacccgctgaccgggcagattgtttccactattccacgttttaccatctacccgaaaacgcactacgtcacaccgcgcgagcgcatcgtacaggcgatggaggagatcaaagaagagctggccgccagacgcaaagtgctgttggaaaacaacaaactgctggaagagcagcggctgacccagcgtacccagtttgatctggagatgatgaacgagctgggctactgttcggggattgaaaactactcgcgcttcctctccggtcgtggaccgggtgagccaccgccgacgctgtttgattacctgcctgccgatgggctgctggtcgtcgatgaatctcacgtcaccattccacaaattggcggcatgtatcgcggtgaccgggcgcgtaaagagacactggtggagtacggcttccgcctgccatcagcgctggataaccgtccgcttaagtttgaagagttcgaagcattagcgccgcaaaccatctatgtttcggcgacgccgggtaattacgagctggaaaaatccggcggcgatgtggtggatcaggtggtgcgtccaaccggattgcttgacccgattatcgaagtgcggccggtggcgacacaggttgatgatcttctttcggagattcgtcagcgagcggcaattaacgaacgcgtactggtcaccacactgaccaagcggatggcggaagatcttaccgaatatctcgaagaacatggcgagcgcgtgcgttatcttcactcagatatcgacaccgtcgaacgtatggagattatccgcgacttgcgtctgggtgagttcgacgtgctggtagggatcaacttactgcgcgaaggtctggatatgccggaagtgtcgctggtggcgatcctcgacgctgacaaagaaggcttcctgcgttccgaacgttcgttgatccagaccattggtcgtgcggcacgtaacgttaacggtaaagcgattctctacggcgataagatcaccccatcaatggcgaaagcgattggcgaaaccgaacgtcgccgtgagaaacagcagaagtacaacgaggaacacggaattacgccgcaaggcttgaacaagaaagtggtcgatatcctggcgctggggcagaacattgccaaaaccaaagcgaagggcagaggaaaatcgcgcccgattgttgagccggataatgtgccgatggatatgtcgcctaaagcgttgcagcagaaaatccatgagctggaagggttgatgatgcaacacgcgcagaatctggagttcgaagaagcggcgcaaattcgtgaccagttgcatcagctgcgtgagctgtttatcgcggcatcgtaa

SEQ ID NO: 17 - DNA, 966 bp, Escherichia coli
atgagaaccgttaaggtattcgaggaagcctggcccttacataccccgtttgtgattgcccggggaagtcgcagtgaagcgcgcgtggtggtggttgaactggaagaagagggtattaaaggcaccggcgaatgcacgccgtatccgcgttatggggaaagtgatgcctcggtaatggcgcaaattatgagcgtcgtgccgcaactagagaaagggctgacacgggaggagttgcaaaaaattctccctgccggcgcagcacgtaatgcgctggattgtgcattgtgggatctggccgcgcgaagacagcagcaatcgctggctgatttgatcggcataacgcttcccgagacagttatcactgcacagacggttgtcatcggtacgcctgatcagatggccaatagtgcatcaacactctggcaggcaggcgcgaaattactgaaagtgaagctggataaccatcttatcagtgagcggatggtggcaattcgcacagctgtgcccgatgcgacgctgatcgttgatgcaaatgaatcctggcgtgcagaagggttggcggcgcgttgccagctattggcggatttaggcgttgcgatgcttgaacaaccgcttcctgcgcaggacgatgcggcgctggagaattttattcatccgttgccgatttgtgctgatgaaagttgtcatactcgtagcaatttgaaggcgctgaaagggcgctatgagatggttaatattaagctcgataaaaccgggggtctgacggaagcgctggcgctggcgactgaagcgcgtgcacaaggtttcagtctgatgctgggctgcatgttgtgtacctctcgtgccattagcgccgctttaccgctggtgccgcaggtcagtttcgccgatcttgacggaccgacctggctggcggtagatgtggaaccggcgcttcagttcacgacgggcgaattgcatctttag

SEQ ID NO: 18 - DNA, 360 bp, Escherichia coli
atgcttcaaatcccacagaattatattcatacgcgctcaacgcctttctggaataaacaaactgcacctgccggaatattcgaacgtcatcttgataaaggaacgcgcccgggggtttacccacgcctttccgttatgcatggggcggtcaaatatctcggctacgctgatgaacacagtgcagagcctgatcaggtgatccttatcgaagcggggcagtttgcggtgttccctccagaaaagtggcacaacattgaagccatgactgacgatacttatttcaacattgacttcttcgtggctcctgaagtcctgatggaaggtgcgcaacaacggaaagtcattcataacgggaaatga

SEQ ID NO: 19 - DNA, 2109 bp, Escherichia coli
ttgtatctgtttgaaagcctgaatcaactgattcaaacctacctgccggaagaccaaatcaagcgtctgcggcaggcgtatctcgttgcacgtgatgctcacgaggggcaaacacgttcaagcggtgaaccctatatcacgcacccggtagcggttgcctgcattctggccgagatgaaactcgactatgaaacgctgatggcggcgctgctgcatgacgtgattgaagatactcccgccacctaccaggatatggaacagctttttggtaaaagcgtcgccgagctggtagagggggtgtcgaaacttgataaactcaagttccgcgataagaaagaggcgcaggccgaaaactttcgcaagatgattatggcgatggtgcaggatatccgcgtcatcctcatcaaacttgccgaccgtacccacaacatgcgcacgctgggctcacttcgcccggacaaacgtcgccgcatcgcccgtgaaactctcgaaatttatagcccgctggcgcaccgtttaggtatccaccacattaaaaccgaactcgaagagctgggttttgaggcgctgtatcccaaccgttatcgcgtaatcaaagaagtggtgaaagccgcgcgcggcaaccgtaaagagatgatccagaagattctttctgaaatcgaagggcgtttgcaggaagcgggaataccgtgccgcgtcagtggtcgcgagaagcatctttattcgatttactgcaaaatggtgctcaaagagcagcgttttcactcgatcatggacatctacgctttccgcgtgatcgtcaatgattctgacacctgttatcgcgtgctgggccagatgcacagcctgtacaagccgcgtccgggccgcgtgaaagactatatcgccattccaaaagcgaacggctatcagtctttgcacacctcgatgatcggcccgcacggtgtgccggttgaggtccagatccgtaccgaagatatggaccagatggcggagatgggtgttgccgcgcactgggcttataaagagcacggcgaaaccagtactaccgcacaaatccgcgcccagcgctggatgcaaagcctgctggagctgcaacagagcgccggtagttcgtttgaatttatcgagagcgttaaatccgatctcttcccggatgagatttacgttttcacaccggaagggcgcattgtcgagctgcctgccggtgcaacgcccgtcgacttcgcttatgcagtgcataccgatatcggtcatgcctgcgtgggcgcacgcgttgaccgccagccttacccgctgtcgcagccgcttaccagcggtcaaaccgttgaaatcattaccgctccgggcgctcgcccgaatgccgcttggctgaactttgtcgttagctcgaaagcgcgcgccaaaattcgtcagttgctgaaaaacctcaagcgtgatgattctgtaagcctgggccgtcgtctgctcaaccatgctttgggtggtagccgtaagctgaatgaaatcccgcaggaaaatattcagcgcgagctggatcgcatgaagctggcaacgcttgacgatctgctggcagaaatcggacttggtaacgcaatgagcgtggtggtcgcgaaaaatctgcaacatggggacgcctccattccaccggcaacccaaagccacggacatctgcccattaaaggtgccgatggcgtgctgatcacctttgcgaaatgctgccgccctattcctggcgacccgattatcgcccacgtcagccccggtaaaggtctggtgatccaccatgaatcctgccgtaatatccgtggctaccagaaagagccagagaagtttatggctgtggaatgggataaagagacggcgcaggagttcatcaccgaaatcaaggtggagatgttcaatcatcagggtgcgctggcaaacctgacggcggcaattaacaccacgacttcgaatattcaaagtttgaatacggaagagaaagatggtcgcgtctacagcgcctttattcgtctgaccgctcgtgaccgtgtgcatctggcgaatatcatgcgcaaaatccgcgtgatgccagacgtgattaaagtcacccgaaaccgaaattaa

SEQ ID NO: 20 - protein, 702 aa, Escherichia coli
MYLFESLNQLIQTYLPEDQIKRLRQAYLVARDAHEGQTRSSGEPYITHPVAVACILAEMKLDYETLMAALLHDVIEDTPATYQDMEQLFGKSVAELVEGVSKLDKLKFRDKKEAQAENFRKMIMAMVQDIRVILIKLADRTHNMRTLGSLRPDKRRRIARETLEIYSPLAHRLGIHHIKTELEELGFEALYPNRYRVIKEVVKAARGNRKEMIQKILSEIEGRLQEAGIPCRVSGREKHLYSIYCKMVLKEQRFHSIMDIYAFRVIVNDSDTCYRVLGQMHSLYKPRPGRVKDYIAIPKANGYQSLHTSMIGPHGVPVEVQIRTEDMDQMAEMGVAAHWAYKEHGETSTTAQIRAQRWMQSLLELQQSAGSSFEFIESVKSDLFPDEIYVFTPEGRIVELPAGATPVDFAYAVHTDIGHACVGARVDRQPYPLSQPLTSGQTVEIITAPGARPNAAWLNFVVSSKARAKIRQLLKNLKRDDSVSLGRRLLNHALGGSRKLNEIPQENIQRELDRMKLATLDDLLAEIGLGNAMSVVVAKNLQHGDASIPPATQSHGHLPIKGADGVLITFAKCCRPIPGDPIIAHVSPGKGLVIHHESCRNIRGYQKEPEKFMAVEWDKETAQEFITEIKVEMFNHQGALANLTAAINTTTSNIQSLNTEEKDGRVYSAFIRLTARDRVHLANIMRKIRVMPDVIKVTRNRN

SEQ ID NO: 21 - DNA, 2352 bp, Escherichia coli
gtggcgcaggcaggttttatcttaacccgacactggcgggacaccccgcaagggacagaagtctccttctggctggcgacggacaacgggccgttgcaggttacgcttgcaccgcaagagtccgtggcgtttattcccgccgatcaggttccccgcgctcagcatattttgcagggtgaacaaggctttcgcctgacaccgctggcgttaaaggattttcaccgccagccggtgtatggcctttactgtcgcgcccatcgccaattgatgaattacgaaaagcgcctgcgtgaaggtggcgttaccgtctacgaggccgatgtgcgtccgccagaacgctatctgatggagcggtttatcacctcaccggtgtgggtcgagggtgatatgcacaatggcactatcgttaatgcccgtctgaaaccgcatcccgactatcgtccgccgctcaagtgggtttctatagatattgaaaccacccgccacggtgagctgtactgcatcggcctggaaggctgcgggcagcgcatcgtttatatgctggggccggagaatggcgacgcctcctcgcttgatttcgaactggaatacgtcgccagccgcccgcagttgctggaaaaactcaacgcctggtttgccaactacgatcctgatgtgatcatcggttggaacgtggtgcagttcgatctgcgaatgctgcaaaaacatgccgagcgttaccgtcttccgctgcgtcttgggcgcgataatagcgagctggagtggcgcgagcacggctttaaaaacggcgtcttttttgcccaggctaaaggtcggctaattatcgacggtatcgaggcgctgaaatccgcgttctggaatttctcttcattctcgctggaaactgtcgctcaggagctattaggcgaaggaaaatctatcgataacccgtgggatcgaatggacgaaattgaccgccgtttcgccgaagataaacctgcgctggcaacttataacctgaaagattgcgagctggtgacgcagatcttccacaaaactgaaatcatgccatttttactcgaacgggcaacggtgaacggcctgccggtggaccgacacggcggttcggtggcggcatttggtcatctctattttccgcgaatgcatcgcgctggttatgtcgcgcctaatctcggcgaagtgccgccgcacgccagccctggcggctacgtgatggattcacggccagggctttatgattcagtgctggtgctggactataaaagcctgtacccgtcgatcatccgcacctttctgattgatcccgtcgggctggtggaaggcatggcgcagcctgatccagagcacagtaccgaaggttttctcgatgcctggttctcgcgagaaaaacattgcctgccggagattgtgactaacatctggcacgggcgcgatgaagccaaacgccagggtaacaaaccgctgtcgcaggcgctgaaaatcatcatgaatgccttttatggcgtgctcggcaccaccgcctgccgcttcttcgatccgcggctggcatcgtcgatcaccatgcgtggtcatcagatcatgcggcaaaccaaagcgttgattgaagcacagggctacgacgttatctacggcgataccgactcaacgtttgtctggctgaaaggcgcacattcggaagaagaagcggcgaaaatcggtcgtgcactggtgcagcacgttaacgcctggtgggcggaaacgctgcaaaaacaacggctgaccagcgcattagaactggagtatgaaacccatttctgccgttttctgatgccaaccattcgcggagccgataccggcagtaaaaagcgttatgccggactgattcaggagggcgacaagcagcggatggtgtttaaagggctggaaaccgtgcgcaccgactggacgccgctggcccagcagtttcagcaggagctatacctgcgcatcttccgcaacgagccatatcaggaatatgtacgcgaaaccatcgacaaactgatggcgggtgaactggatgcgcgactggtttaccgtaaacgccttcgccgtccgctgagcgagtatcagcgtaatgtgccgcctcatgtacgcgccgctcgccttgccgatgaagaaaaccaaaagcgtggtcgccccttgcaatatcagaatcgcggcaccattaagtacgtatggaccaccaacggcccggagccgctggactaccaacgttcaccactggattacgaacactatctgacccgccagctacaacccgtggcggagggaatactcccttttattgaggataattttgctacacttatgaccgggcaacttgggctattttga

SEQ ID NO: 22 - protein, 783 aa, Escherichia coli
MAQAGFILTRHWRDTPQGTEVSFWLATDNGPLQVTLAPQESVAFIPADQVPRAQHILQGEQGFRLTPLALKDFHRQPVYGLYCRAHRQLMNYEKRLREGGVTVYEADVRPPERYLMERFITSPVWVEGDMHNGTIVNARLKPHPDYRPPLKWVSIDIETTRHGELYCIGLEGCGQRIVYMLGPENGDASSLDFELEYVASRPQLLEKLNAWFANYDPDVIIGWNVVQFDLRMLQKHAERYRLPLRLGRDNSELEWREHGFKNGVFFAQAKGRLIIDGIEALKSAFWNFSSFSLETVAQELLGEGKSIDNPWDRMDEIDRRFAEDKPALATYNLKDCELVTQIFHKTEIMPFLLERATVNGLPVDRHGGSVAAFGHLYFPRMHRAGYVAPNLGEVPPHASPGGYVMDSRPGLYDSVLVLDYKSLYPSIIRTFLIDPVGLVEGMAQPDPEHSTEGFLDAWFSREKHCLPEIVTNIWHGRDEAKRQGNKPLSQALKIIMNAFYGVLGTTACRFFDPRLASSITMRGHQIMRQTKALIEAQGYDVIYGDTDSTFVWLKGAHSEEEAAKIGRALVQHVNAWWAETLQKQRLTSALELEYETHFCRFLMPTIRGADTGSKKRYAGLIQEGDKQRMVFKGLETVRTDWTPLAQQFQQELYLRIFRNEPYQEYVRETIDKLMAGELDARLVYRKRLRRPLSEYQRNVPPHVRAARLADEENQKRGRPLQYQNRGTIKYVWTTNGPEPLDYQRSPLDYEHYLTRQLQPVAEGILPFIEDNFATLMTGQLGLF

SEQ ID NO: 23 - DNA, 4029 bp, Escherichia coli
atggtttactcctataccgagaaaaaacgtattcgtaaggattttggtaaacgtccacaagttctggatgtaccttatctcctttctatccagcttgactcgtttcagaaatttatcgagcaagatcctgaagggcagtatggtctggaagctgctttccgttccgtattcccgattcagagctacagcggtaattccgagctgcaatacgtcagctaccgccttggcgaaccggtgtttgacgtccaggaatgtcaaatccgtggcgtgacctattccgcaccgctgcgcgttaaactgcgtctggtgatctatgagcgcgaagcgccggaaggcaccgtaaaagacattaaagaacaagaagtctacatgggcgaaattccgctcatgacagacaacggtacctttgttatcaacggtactgagcgtgttatcgtttcccagctgcaccgtagtccgggcgtcttctttgactccgacaaaggtaaaacccactcttcgggtaaagtgctgtataacgcgcgtatcatcccttaccgtggttcctggctggacttcgaattcgatccgaaggacaacctgttcgtacgtatcgaccgtcgccgtaaactgcctgcgaccatcattctgcgcgccctgaactacaccacagagcagatcctcgacctgttctttgaaaaagttatctttgaaatccgtgataacaagctgcagatggaactggtgccggaacgcctgcgtggtgaaaccgcatcttttgacatcgaagctaacggtaaagtgtacgtagaaaaaggccgccgtatcactgcgcgccacattcgccagctggaaaaagacgacgtcaaactgatcgaagtcccggttgagtacatcgcaggtaaagtggttgctaaagactatattgatgagtctaccggcgagctgatctgcgcagcgaacatggagctgagcctggatctgctggctaagctgagccagtctggtcacaagcgtatcgaaacgctgttcaccaacgatctggatcacggcccatatatctctgaaaccttacgtgtcgacccaactaacgaccgtctgagcgcactggtagaaatctaccgcatgatgcgccctggcgagccgccgactcgtgaagcagctgaaagcctgttcgagaacctgttcttctccgaagaccgttatgacttgtctgcggttggtcgtatgaagttcaaccgttctctgctgcgcgaagaaatcgaaggttccggtatcctgagcaaagacgacatcattgatgttatgaaaaagctcatcgatatccgtaacggtaaaggcgaagtcgatgatatcgaccacctcggcaaccgtcgtatccgttccgttggcgaaatggcggaaaaccagttccgcgttggcctggtacgtgtagagcgtgcggtgaaagagcgtctgtctctgggcgatctggataccctgatgccacaggatatgatcaacgccaagccgatttccgcagcagtgaaagagttcttcggttccagccagctgtctcagtttatggaccagaacaacccgctgtctgagattacgcacaaacgtcgtatctccgcactcggcccaggcggtctgacccgtgaacgtgcaggcttcgaagttcgagacgtacacccgactcactacggtcgcgtatgtccaatcgaaacccctgaaggtccgaacatcggtctgatcaactctctgtccgtgtacgcacagactaacgaatacggcttccttgagactccgtatcgtaaagtgaccgacggtgttgtaactgacgaaattcactacctgtctgctatcgaagaaggcaactacgttatcgcccaggcgaactccaacttggatgaagaaggccacttcgtagaagacctggtaacttgccgtagcaaaggcgaatccagcttgttcagccgcgaccaggttgactacatggacgtatccacccagcaggtggtatccgtcggtgcgtccctgatcccgttcctggaacacgatgacgccaaccgtgcattgatgggtgcgaacatgcaacgtcaggccgttccgactctgcgcgctgataagccgctggttggtactggtatggaacgtgctgttgccgttgactccggtgtaactgcggtagctaaacgtggtggtgtcgttcagtacgtggatgcttcccgtatcgttatcaaagttaacgaagacgagatgtatccgggtgaagcaggtatcgacatctacaacctgaccaaatacacccgttctaaccagaacacctgtatcaaccagatgccgtgtgtgtctctgggtgaaccggttgaacgtggcgacgtgctggcagacggtccgtccaccgacctcggtgaactggcgcttggtcagaacatgcgcgtagcgttcatgccgtggaatggttacaacttcgaagactccatcctcgtatccgagcgtgttgttcaggaagaccgtttcaccaccatccacattcaggaactggcgtgtgtgtcccgtgacaccaagctgggtccggaagagatcaccgctgacatcccgaacgtgggtgaagctgcgctctccaaactggatgaatccggtatcgtttacattggtgcggaagtgaccggtggcgacattctggttggtaaggtaacgccgaaaggtgaaactcagctgaccccagaagaaaaactgctgcgtgcgatcttcggtgagaaagcctctgacgttaaagactcttctctgcgcgtaccaaacggtgtatccggtacggttatcgacgttcaggtctttactcgcgatggcgtagaaaaagacaaacgtgcgctggaaatcgaagaaatgcagctcaaacaggcgaagaaagacctgtctgaagaactgcagatcctcgaagcgggtctgttcagccgtatccgtgctgtgctggtagccggtggcgttgaagctgagaagctcgacaaactgccgcgcgatcgctggctggagctgggcctgacagacgaagagaaacaaaatcagctggaacagctggctgagcagtatgacgaactgaaacacgagttcgagaagaaactcgaagcgaaacgccgcaaaatcacccagggcgacgatctggcaccgggcgtgctgaagattgttaaggtatatctggcggttaaacgccgtatccagcctggtgacaagatggcaggtcgtcacggtaacaagggtgtaatttctaagatcaacccgatcgaagatatgccttacgatgaaaacggtacgccggtagacatcgtactgaacccgctgggcgtaccgtctcgtatgaacatcggtcagatcctcgaaacccacctgggtatggctgcgaaaggtatcggcgacaagatcaacgccatgctgaaacagcagcaagaagtcgcgaaactgcgcgaattcatccagcgtgcgtacgatctgggcgctgacgttcgtcagaaagttgacctgagtaccttcagcgatgaagaagttatgcgtctggctgaaaacctgcgcaaaggtatgccaatcgcaacgccggtgttcgacggtgcgaaagaagcagaaattaaagagctgctgaaacttggcgacctgccgacttccggtcagatccgcctgtacgatggtcgcactggtgaacagttcgagcgtccggtaaccgttggttacatgtacatgctgaaactgaaccacctggtcgacgacaagatgcacgcgcgttccaccggttcttacagcctggttactcagcagccgctgggtggtaaggcacagttcggtggtcagcgtttcggggagatggaagtgtgggcgctggaagcatacggcgcagcatacaccctgcaggaaatgctcaccgttaagtctgatgacgtgaacggtcgtaccaagatgtataaaaacatcgtggacggcaaccatcagatggagccgggcatgccagaatccttcaacgtattgttgaaagagattcgttcgctgggtatcaacatcgaactggaagacgagtaa

SEQ ID NO: 24 - protein, 1342 aa, Escherichia coli [sequence omitted]

SEQ ID NO: 25 - DNA, 4224 bp, Escherichia coli
gtgaaagatttattaaagtttctgaaagcgcagactaaaaccgaagagtttgatgcgatcaaaattgctctggcttcgccagacatgatccgttcatggtctttcggtgaagttaaaaagccggaaaccatcaactaccgtacgttcaaaccagaacgtgacggccttttctgcgcccgtatctttgggccggtaaaagattacgagtgcctgtgcggtaagtacaagcgcctgaaacaccgtggcgtcatctgtgagaagtgcggcgttgaagtgacccagactaaagtacgccgtgagcgtatgggccacatcgaactggcttccccgactgcgcacatctggttcctgaaatcgctgccgtcccgtatcggtctgctgctcgatatgccgctgcgcgatatcgaacgcgtactgtactttgaatcctatgtggttatcgaaggcggtatgaccaacctggaacgtcagcagatcctgactgaagagcagtatctggacgcgctggaagagttcggtgacgaattcgacgcgaagatgggggcggaagcaatccaggctctgctgaagagcatggatctggagcaagagtgcgaacagctgcgtgaagagctgaacgaaaccaactccgaaaccaagcgtaaaaagctgaccaagcgtatcaaactgctggaagcgttcgttcagtctggtaacaaaccagagtggatgatcctgaccgttctgccggtactgccgccagatctgcgtccgctggttccgctggatggtggtcgtttcgcgacttctgacctgaacgatctgtatcgtcgcgtcattaaccgtaacaaccgtctgaaacgtctgctggatctggctgcgccggacatcatcgtacgtaacgaaaaacgtatgctgcaggaagcggtagacgccctgctggataacggtcgtcgcggtcgtgcgatcaccggttctaacaagcgtcctctgaaatctttggccgacatgatcaaaggtaaacagggtcgtttccgtcagaacctgctcggtaagcgtgttgactactccggtcgttctgtaatcaccgtaggtccatacctgcgtctgcatcagtgcggtctgccgaagaaaatggcactggagctgttcaaaccgttcatctacggcaagctggaactgcgtggtcttgctaccaccattaaagctgcgaagaaaatggttgagcgcgaagaagctgtcgtttgggatatcctggacgaagttatccgcgaacacccggtactgctgaaccgtgcaccgactctgcaccgtctgggtatccaggcatttgaaccggtactgatcgaaggtaaagctatccagctgcacccgctggtttgtgcggcatataacgccgacttcgatggtgaccagatggctgttcacgtaccgctgacgctggaagcccagctggaagcgcgtgcgctgatgatgtctaccaacaacatcctgtccccggcgaacggcgaaccaatcatcgttccgtctcaggacgttgtactgggtctgtactacatgacccgtgactgtgttaacgccaaaggcgaaggcatggtgctgactggcccgaaagaagcagaacgtctgtatcgctctggtctggcttctctgcatgcgcgcgttaaagtgcgtatcaccgagtatgaaaaagatgctaacggtgaattagtagcgaaaaccagcctgaaagacacgactgttggccgtgccattctgtggatgattgtaccgaaaggtctgccttactccatcgtcaaccaggcgctgggtaaaaaagcaatctccaaaatgctgaacacctgctaccgcattctcggtctgaaaccgaccgttatttttgcggaccagatcatgtacaccggcttcgcctatgcagcgcgttctggtgcatctgttggtatcgatgacatggtcatcccggagaagaaacacgaaatcatctccgaggcagaagcagaagttgctgaaattcaggagcagttccagtctggtctggtaactgcgggcgaacgctacaacaaagttatcgatatctgggctgcggcgaacgatcgtgtatccaaagcgatgatggataacctgcaaactgaaaccgtgattaaccgtgacggtcaggaagagaagcaggtttccttcaacagcatctacatgatggccgactccggtgcgcgtggttctgcggcacagattcgtcagcttgctggtatgcgtggtctgatggcgaagccggatggctccatcatcgaaacgccaatcaccgcgaacttccgtgaaggtctgaacgtactccagtacttcatctccacccacggtgctcgtaaaggtctggcggataccgcactgaaaactgcgaactccggttacctgactcgtcgtctggttgacgtggcgcaggacctggtggttaccgaagacgattgtggtacccatgaaggtatcatgatgactccggttatcgagggtggtgacgttaaagagccgctgcgcgatcgcgtactgggtcgtgtaactgctgaagacgttctgaagccgggtactgctgatatcctcgttccgcgcaacacgctgctgcacgaacagtggtgtgacctgctggaagagaactctgtcgacgcggttaaagtacgttctgttgtatcttgtgacaccgactttggtgtatgtgcgcactgctacggtcgtgacctggcgcgtggccacatcatcaacaagggtgaagcaatcggtgttatcgcggcacagtccatcggtgaaccgggtacacagctgaccatgcgtacgttccacatcggtggtgcggcatctcgtgcggctgctgaatccagcatccaagtgaaaaacaaaggtagcatcaagctcagcaacgtgaagtcggttgtgaactccagcggtaaactggttatcacttcccgtaatactgaactgaaactgatcgacgaattcggtcgtactaaagaaagctacaaagtaccttacggtgcggtactggcgaaaggcgatggcgaacaggttgctggcggcgaaaccgttgcaaactgggacccgcacaccatgccggttatcaccgaagtaagcggttttgtacgctttactgacatgatcgacggccagaccattacgcgtcagaccgacgaactgaccggtctgtcttcgctggtggttctggattccgcagaacgtaccgcaggtggtaaagatctgcgtccggcactgaaaatcgttgatgctcagggtaacgacgttctgatcccaggtaccgatatgccagcgcagtacttcctgccgggtaaagcgattgttcagctggaagatggcgtacagatcagctctggtgacaccctggcgcgtattccgcaggaatccggcggtaccaaggacatcaccggtggtctgccgcgcgttgcggacctgttcgaagcacgtcgtccgaaagagccggcaatcctggctgaaatcagcggtatcgtttccttcggtaaagaaaccaaaggtaaacgtcgtctggttatcaccccggtagacggtagcgatccgtacgaagagatgattccgaaatggcgtcagctcaacgtgttcgaaggtgaacgtgtagaacgtggtgacgtaatttccgacggtccggaagcgccgcacgacattctgcgtctgcgtggtgttcatgctgttactcgttacatcgttaacgaagtacaggacgtataccgtctgcagggcgttaagattaacgataaacacatcgaagttatcgttcgtcagatgctgcgtaaagctaccatcgttaacgcgggtagctccgacttcctggaaggcgaacaggttgaatactctcgcgtcaagatcgcaaaccgcgaactggaagcgaacggcaaagtgggtgcaacttactcccgcgatctgctgggtatcaccaaagcgtctctggcaaccgagtccttcatctccgcggcatcgttccaggagaccactcgcgtgctgaccgaagcagccgttgcgggcaaacgcgacgaactgcgcggcctgaaagagaacgttatcgtgggtcgtctgatcccggcaggtaccggttacgcgtaccaccaggatcgtatgcgtcgccgtgctgcgggtgaagctccggctgcaccgcaggtgactgcagaagacgcatctgccagcctggcagaactgctgaacgcaggtctgggcggttctgataacgagtaa

SEQ ID NO: 26 - protein, 1407 aa, Escherichia coli [sequence omitted]

SEQ ID NO: 27 - DNA, 648 bp, Escherichia coli
atgtccgataacgctcaacttaccggtctgtgcgaccgttttcgtggtttttatcctgttgtgatcgatgttgaaacagccggatttaacgccaaaaccgatgcgctgcttgagattgccgccatcaccctgaaaatggatgaacaaggctggctgatgccggacaccacattacatttccacgtcgaaccatttgtcggcgcaaatttgcaaccagaagccctcgccttcaacggcattgacccgaacgatcccgatcgcggcgcggtcagcgaatacgaggcgctgcacgaaatttttaaagttgtacgtaaaggtattaaagcgagcggctgtaaccgcgccattatggtggcgcacaatgccaattttgatcacagctttatgatggccgccgcagaacgcgcctcactgaaacgtaacccgttccaccctttcgccacttttgacactgctgcactggccgggctggcactcggacaaaccgtattgtcaaaggcttgccagaccgctggcatggacttcgacagcacccaggcgcactccgcgctgtacgacaccgaacgcactgctgtgctgttttgtgaaatcgtcaaccgctggaaacgtctgggaggctggccgctatctgccgccgaagaggtgtaa

SEQ ID NO: 28 - protein, 215 aa, Escherichia coli
MSDNAQLTGLCDRFRGFYPVVIDVETAGFNAKTDALLEIAAITLKMDEQGWLMPDTTLHFHVEPFVGANLQPEALAFNGIDPNDPDRGAVSEYEALHEIFKVVRKGIKASGCNRAIMVAHNANFDHSFMMAAAERASLKRNPFHPFATFDTAALAGLALGQTVLSKACQTAGMDFDSTQAHSALYDTERTAVLFCEIVNRWKRLGGWPLSAAEEV

SEQ ID NO: 29 - DNA, 891 bp, Escherichia coli
atgccttacgatagcgtatacagcgaaaagcgcccgccgggcacgctgcgtaccgcctggcgcaaattttatagtgacgcctctgcgatggtcggcctgtacggctgcgcggggctggctgtactgtgtatttttggcggctggtttgcgccttacggcatcgaccagcaatttctcggttatcaattattgccgccgtcatggtcgcgctatggcgaagtttctttcttcctggggactgacgacctcggacgcgatgttttaagccgtttactgagcggagctgcgccaaccgtgggtggcgcatttgtggtgacgcttgccgcgacgatctgtggcctggtactgggcaccttcgccggggcgacacacggcctccgctcagcggtgcttaaccatattctggataccttactggcgatcccttcgctgctgttggcaattatcgttgtagcgtttgccggaccgagtttgtcccacgctatgtttgctgtctggctggcattgctgccgcgtatggtacgttcgatttacagcatggtgcatgacgaactggaaaaagagtacgttatcgccgcccgtctggatggcgcatcaacgctgaatattctctggtttgctgtgatgccaaacatcaccgccgggctggtcaccgagatcacccgcgcactgtcgatggcaattctcgatatcgccgcgctgggctttctcgatctcggcgcacaactcccctcgcctgaatggggagcgatgctcggtgatgcgctggaactgatttatgtcgccccgtggactgtcatgctgccaggtgcggcaattatgattagcgtgttgttggttaacctccttggcgatggcgttcgtcgtgcgattattgcgggggtggaataa

SEQ ID NO: 30 - protein, 296 aa, Escherichia coli
MPYDSVYSEKRPPGTLRTAWRKFYSDASAMVGLYGCAGLAVLCIFGGWFAPYGIDQQFLGYQLLPPSWSRYGEVSFFLGTDDLGRDVLSRLLSGAAPTVGGAFVVTLAATICGLVLGTFAGATHGLRSAVLNHILDTLLAIPSLLLAIIVVAFAGPSLSHAMFAVWLALLPRMVRSIYSMVHDELEKEYVIAARLDGASTLNILWFAVMPNITAGLVTEITRALSMAILDIAALGFLDLGAQLPSPEWGAMLGDALELIYVAPWTVMLPGAAIMISVLLVNLLGDGVRRAIIAGVE

SEQ ID NO: 31 - DNA, 642 bp, Escherichia coli
atgaaaccatatcagcgccagtttattgaatttgcgcttagcaagcaggtgttaaagtttggcgagtttacgctgaaatccgggcgcaaaagcccctatttcttcaacgccgggctgtttaataccgggcgcgatctggcactgttaggccgtttttacgctgaagcgttggtggattccggcattgagttcgatctgctgtttggccctgcttacaaagggatcccgattgccaccacaaccgctgtggcactggcggagcatcacgacctggacctgccgtactgctttaaccgcaaagaagcaaaagaccacggtgaaggcggcaatctggttggtagcgcgttacaaggacgcgtaatgctggtagatgatgtgatcaccgccggaacggcgattcgcgagtcgatggagattattcaggccaatggcgcgacgcttgctggcgtgttgatttcgctcgatcgtcaggaacgcgggcgcggcgagatttcggcgattcaggaagttgagcgtgattacaactgcaaagtgatctctatcatcaccctgaaagacctgattgcttacctggaagagaagccggaaatggcggaacatctggcggcggttaaggcctatcgcgaagagtttggcgtttaa

SEQ ID NO: 32 - protein, 213 aa, Escherichia coli
MKPYQRQFIEFALSKQVLKFGEFTLKSGRKSPYFFNAGLFNTGRDLALLGRFYAEALVDSGIEFDLLFGPAYKGIPIATTTAVALAEHHDLDLPYCFNRKEAKDHGEGGNLVGSALQGRVMLVDDVITAGTAIRESMEIIQANGATLAGVLISLDRQERGRGEISAIQEVERDYNCKVISIITLKDLIAYLEEKPEMAEHLAAVKAYREEFGV

SEQ ID NO: 33 - DNA, 65 bp, Escherichia coli
gccttcgctcctcatcttacttttctacagacaaaaaaaaggcgactcatcagtcgccttaaaaa

SEQ ID NO: 34 - DNA, 891 bp, Escherichia coli
atggctatggttgaaatgcaaaccactgcggcgctggctgaatctcgccgcaaaatgcaggcgcgtcgccgcctcaaaaaccgtattgcgctgacgctctcgatggcgacgatggccttcggcctgttctggctgatctggattttaatgtccaccatcactcgcggtatcgacggtatgtcgctggcgctgttcactgaaatgacgccgccgcccaatacggaaggtggtggtctggcgaacgctctggcgggtagcgggctgttaattttgtgggccacggtattcggtacgccgctgggcattatggcggggatttatctggcggaatatggtcgtaaatcctggctggcagaagtgattcgcttcattaacgacattctgctctctgcgccgtcgattgtggttggtctgtttgtttacaccattgtggtggcgcagatggagcacttctccggctgggcgggcgtgattgccctggcgttgttgcaggtgccgattgttatccgcaccaccgagaacatgctgaaactggtgccgtacagcctgcgtgaagcggcttatgcgctgggtacaccgaagtggaagatgatctctgcgattacgctgaaagcgtcggtgtccgggattatgaccggtatcctgctggcgattgcccgtattgctggtgaaaccgcgccgctgctgtttaccgcgctctccaaccagttctggagcacggacatgatgcagccgatcgccaacctgccggtgacgatctttaagtttgcgatgagcccgtttgcggaatggcagcaattggcctgggccggggtattgatcattaccctgtgcgtactgctgctgaacattctggcgcgcgttgtttttgcgaagaataaacacggttga

SEQ ID NO: 35 - DNA, 774 bp, Escherichia coli
atgagtatggttgaaactgccccgagtaaaattcaggttcgtaatttgaacttctactacggcaaattccatgccctgaaaaacatcaacctggatatcgctaaaaaccaggtaacggcgtttatcgggccgtccggctgcggtaaatcgacgctgctgcgtaccttcaacaaaatgtttgaactgtacccggagcagcgtgcggaaggtgaaattctgcttgatggcgacaacatcctgaccaactctcaggatatcgcactgctgcgtgcgaaagtgggcatggtgttccagaaaccgacgccgtttccgatgtccatctacgacaacatcgcttttggcgttcgtctgtttgagaagctctcccgtgccgacatggacgagcgcgtgcagtgggcattgaccaaagccgcattgtggaacgaaaccaaagataaattgcaccagagcggttactctctctctggtggtcagcaacagcgtctgtgtattgcgcgtggtatcgccattcgcccggaagtgctgctgctcgacgaaccgtgttcggcgctcgaccctatctctaccgggcgtattgaagagctgatcaccgaactgaagcaggattacaccgtggtgatcgtcacccacaacatgcagcaggctgcgcgttgttccgaccacacggcgtttatgtacctgggcgaattgattgagttcagcaacacggacgatctgttcaccaagccagcgaagaaacaaacagaagactacatcaccggtcgttacggttga

SEQ ID NO: 36 - DNA, 960 bp, Escherichia coli
atggctgcaaccaagcctgcttttaacccaccgggtaaaaagggcgacataattttcagcgtgctggtaaaactggcggcgctgattgtgctattgatgttgggtggcattattgtctctctgatcatctcctcctggccgagcattcagaaatttggtctggctttcctatggaccaaagagtgggatgcaccgaacgatatctacggggcgctggtgccgatctacggtacgttggtgacttcgtttatcgcgctgctgatcgccgtcccggtgagtttcggtatcgccctgttcctgactgagcttgcgcctggctggctgaaacgcccgctgggtatcgccattgagctgctggcagccattccaagtatcgtttacggcatgtggggcctgtttatctttgcgccgctgttcgccgtttactttcaggagccggtcggcaatatcatgtcgaatatcccgattgttggcgcgctgttctctggccccgcatttggtatcggtatcctcgcggcaggcgtgatcctcgccatcatgattattccgtacattgcggcggtaatgcgtgatgtgttcgaacaaaccccggtgatgatgaaagagtcggcctacggtattggctgcaccacctgggaagttatctggcgtatcgttcttccgttcaccaaaaatggtgttatcggcggcatcatgctggggctgggccgcgcgctcggtgaaaccatggcggtgacctttatcatcggtaacacctaccagctcgacagcgcctcgctgtatatgccgggcaacagtatcacctctgcgctggcgaacgaatttgcggaagcggaatccggtctgcacgttgccgcactgatggaactgggcctgatcctgtttgtgattaccttcatcgtcctcgccgcatcgaagtttatgattatgcgcctggctaagaatgagggggcacgctaa

(19) United States
(12) Patent Application Publication
(10) Pub. No.: US 2023/0139445 A1
(43) Pub. Date: May 4, 2023

(54) BACTERIAL CELLS WITH IMPROVED TOLERANCE TO DIACIDS

(71) Applicant: DANMARKS TEKNISKE UNIVERSITET, Kgs. Lyngby (DK)

(72) Inventors: Rebecca Lennen, Holte (DK); Alex Toftgaard Nielsen, Rungsted Kyst (DK); Markus Herrgård, Virum (DK); Morten Sommer, Hellerup (DK); Adam Feist, San Diego, CA (US); Elsayed Tharwat Tolba Mohamed, Helsingborg (SE)

(21) Appl. No.: 16/461,170

(22) PCT Filed: Nov. 15, 2017
(86) PCT No.: PCT/EP2017/079313

§ 371 Date: May 15, 2019

Related U.S. Application Data
(60) Provisional application No. 62/422,457, filed on Nov. 15, 2016.

(30) Foreign Application Priority Data
Nov. 30, 2016 (EP) 16201310.6

(51) Int. Cl. C12P 7/46; C12N 1/20; C12N 15/52; C07K 14/245

(52) U.S. Cl. CPC C12P 7/46; C12N 1/20; C12N 15/52; C07K 14/245; C12N 2330/50

(57) ABSTRACT

The present invention relates to bacterial cells genetically modified to improve their tolerance to certain commodity chemicals, such as diacids, and to methods of preparing and using such bacterial cells for production of diacids and other compounds.

### FIELD OF THE INVENTION

The present invention relates to bacterial cells genetically modified to improve their tolerance to certain commodity chemicals, such as dicarboxylic acids (herein referred to as “diacids”) and other polycarboxylic acids, and to methods of preparing and using such bacterial cells for production of diacids and other compounds.

### BACKGROUND OF THE INVENTION

Aliphatic diacids are commonly used as precursors for nylon polymers (polyamides), typically prepared by condensing diamines with diacids. Diacids are also used as monomers for various other polymers and copolymers including polyurethanes. Different chain lengths and the presence of unsaturated bonds or branched chains within the constituent diacids imparts different physical properties to the polymer.

There has been significant recent interest in producing diacids biologically, i.e., in microbial cells. For example, as reported on their respective websites (accessed in October 2016), Myriant Corporation and BioAmber Inc. have both begun biological production of succinic acid, as a replacement molecule for petrochemical-derived adipic acid, Verdezyne Inc. is developing a process to produce adipic acid in yeast, and one of the major worldwide manufacturers of nylons, INVISTA™, is actively seeking the development of biologically produced precursors through collaborations with external parties. The production of diacids in metabolically engineered microbial cells have been reviewed and described in several publications such as, e.g., Polen et al., 2013; Adkins et al., 2013; Park et al., 2013; Yu et al., 2014; Cheong et al., 2016; Deng and Mao, 2015; WO 2011/003034 A2 (Verdezyne); Curran et al., 2013; Sengupta et al., 2015; and Zhang et al., 2015.

For production of bulk chemicals from renewable plant-based carbon feedstocks, high product titers are essential in order to minimize capital equipment and downstream separations costs for product purification. At the high titers required for economical fermentation processes, however, most chemicals exhibit significant toxicity that reduces yields and productivities by negatively affecting microbial growth (Van Dien, 2013; Zingaro et al., 2013). Escherichia coli being a suitable host for industrial applications, there has been some interest in developing E. coli strains with improved tolerance to chemicals of interest for production, such as, e.g., n-butanol, ethanol and isobutanol, or to stress conditions present during fermentation (see, e.g., Haft et al, 2014; Sandberg et al., 2014; Lennen and Herrgard, 2014; Tenaillon et al., 2012; Minty et al., 2011; Dragosits et al., 2013a,b; Winkler et al., 2014; Wu et al., 2014; LaCroix et al., 2015; Jensen et al., 2015 and 2016; Doukyu et al., 2012; Shenhar et al., 2012; and Rath and Jawali, 2006).

In addition, Byrne et al., 2012, describes computational modelling of microorganisms such as E. coli, proposing combinations of medium compositions and gene-deletion strains for six industrially important byproducts, e.g., succinate. WO 01/05959 (Ajinomoto K K) relates to production of a target substance such as glutamic acid in, e.g., E. coli strains. Finally, WO 2016/162442 (Metabolic Explorer) relates to a recombinant microorganism capable of producing 2,4-dihydroxybutyrate, which is characterized by an increased cellular export, and preferably by a decreased cellular import, of 2,4-dihydroxybutyrate.

Despite these and other advances in the art, there is still a need for bacterial cells with improved tolerance to chemicals of interest for bio-based production, such as aliphatic diacids and other compounds. It is an object of the invention to provide such bacterial cells.

### SUMMARY OF THE INVENTION

It has been found by the present inventors that certain genetic modifications unexpectedly improve the tolerance of bacterial cells, such as those of, e.g., the Escherichia genera, to certain chemical compounds, particularly aliphatic diacids (herein also referred to as “aliphatic dicarboxylic acids”).

Accordingly, the invention provides bacterial cells with improved tolerance to at least one aliphatic diacid, as well as bacterial cells which are capable of producing an aliphatic diacid and have improved tolerance to the aliphatic diacid. Particularly contemplated are glutaric acid, adipic acid, succinic acid, muconic acid, fumaric acid, itaconic acid, malic acid, malonic acid, maleic acid, glucaric acid, pimelic acid, suberic acid, sebacic acid, 2,5-furandicarboxylic acid, terephthalic acid, azelaic acid, mesaconic acid, citraconic acid, tartaric acid, tartronic acid, diaminopimelic acid, and glutaconic acid.

The invention also relates to compositions comprising such bacterial cells and one or more aliphatic diacids, methods of preparing or screening for such bacterial cells, and methods of producing aliphatic diacids using such bacterial cells.

These and other aspects and embodiments are described in more detail below.

### DETAILED DISCLOSURE OF THE INVENTION

In the present work, glutaric acid and adipic acid were selected for performing adaptive laboratory evolutions. Based on the findings reported herein, various aspects of the invention provide for genetically modified bacterial host cells with a higher tolerance to one or more diacids or other compounds. When transformed with a recombinant biosynthetic pathway for producing the diacid from a carbon source, the genetically modified bacterial host cells of the invention result in improved production of the diacid from carbon feedstock, since they maintain robust metabolic activity in the presence of higher concentrations of the diacid than the parent cells. For example, it was found that a reduced expression of kgtP improved tolerance to glutaric acid, implicating KgtP, an α-ketoglutarate importer, as being a direct importer for glutarate.

So, in a first aspect, a bacterial cell is provided, comprising a biosynthetic pathway for producing an aliphatic dicarboxylic acid and at least one genetic modification which reduces expression of an endogenous gene selected from the group consisting of kgtP, ybjL, proV, proW, proX, proQ, cspE, rfaE, yfbP, yfjM, pstS, pstA, pstB, pstC, rph, rpoS, sspA, tdk, uvrB, ycjG, and yeaR, or a combination of any thereof. In one preferred embodiment, the genetic modification reduces the expression of kgtP. In another preferred embodiment, the at least one genetic modification reduces the expression of ybjL, proV, proW, proX, sspA or a combination of any thereof. For example, the bacterial cell may comprise genetic modifications which reduce the expression of kgtP, proV and ybjL; kgtP and proV; kgtP and ybjL; or kgtP and sspA. Non-limiting examples of genetic modifications include a knock-down or knock-out of the endogenous gene. In a particular embodiment, the genetic modification is a knock-out. The genetic modification may, for example, provide for an increased growth rate, a reduced lag time, or both, of the cell in the presence of at least one of glutaric acid and adipic acid as compared to a control, e.g., the bacterial cell without the genetic modification.

In a second aspect, a bacterial cell is provided, genetically modified from a parent bacterial cell so as to comprise one or more of
(a) a mutant SpoT, comprising at least one mutation in the threonyl-tRNA synthetase GTPase and SpoT (TGS) domain corresponding to amino acid residues 1388 to T447 and/or the linker segment between the TGS and the aspartokinase, chorismate mutase and TyrA (ACT) domain corresponding to amino acid residues A448 to T621, optionally in one or more amino acid residues selected from A451, R236, V422, W457, N454, D580, M247, T442, S434, N601, 1602 and R603;
(b) a mutant PolB, comprising a mutation in amino acid residue R477;
(c) a mutant RpoC, comprising a mutation in at least one of the amino acid residues corresponding to H419 and P64;
(d) a mutant RpoB, comprising a mutation in an amino acid residue corresponding to K203;
(e) a mutant Rnt, comprising a mutation in at least one of the amino acid residues corresponding to Q179, A27, F194 and A180;
(f) a mutant SapC, comprising a mutation in the amino acid residue corresponding to G79; and
(g) increased expression of PyrE as compared to the parent bacterial cell;
   optionally in combination with a knock-down or knock-out of (i) at least one endogenous gene selected from the group consisting of kgtP, ybjL, proV, proW, proX, proQ, cspE, rfaE, yfbP, yfjM, pstS, pstA, pstB, pstC, rph, rpoS, sspA, tdk, uvrB, ycjG, and yeaR; or (ii) a combination of two or more endogenous genes selected from kgtP, proV, ybjL and sspA, such as kgtP, proV and ybjL; kgtP and proV; kgtP and ybjL; and kgtP and sspA,
   wherein the genetic modification provides for an increased growth rate, a reduced lag time, or both, in the presence of at least one of glutaric acid and adipic acid as compared to the parent bacterial cell.

The bacterial cell, may, for example, comprise (a) at least one mutant protein selected from the group consisting of SpoT-V422A, SpoT-A451D, SpoT-A451V, SpoT-W457C, SpoT-N454H, SpoT-D580Y, SpoT-R236L, SpoT-R236S, SpoT-M247K, SpoT-NIR(601-603)S, SpoT-T442I, SpoT-S434L, PolB-R477G, RpoC-H419P, RpoC-P64L, RpoB-K203T, Rnt-Q179P, Rnt-A27T, Rnt-F194L, Rnt-A180T, SapC-G79W; and/or (b) a mutation in rph or the pyrE/rph intergenic region which increases the expression of PyrE.

The bacterial cell of any aspect or embodiment may further comprise a recombinant biosynthetic pathway for producing at least one of glutaric acid, adipic acid, succinic acid, muconic acid, fumaric acid, itaconic acid, malic acid, malonic acid, maleic acid, glucaric acid, pimelic acid, suberic acid, sebacic acid, 2,5-furandicarboxylic acid, terephthalic acid, or azelaic acid, mesaconic acid, citraconic acid, tartaric acid, tartronic acid, diaminopimelic acid and glutaconic acid.

Also provided is a process for preparing a recombinant bacterial cell for producing an aliphatic dicarboxylic acid, the process comprising genetically modifying an E. coli cell to (a) introduce a recombinant biosynthetic pathway for producing the aliphatic dicarboxylic acid, and (b) knock-down or knock-out at least one endogenous gene selected from the group consisting of kgtP, ybjL, proV, proW, proX, proQ, cspE, rfaE, yfbP, yfjM, pstS, pstA, pstB, pstC, rph, rpoS, sspA, tdk, uvrB, ycjG, and yeaR; such as a combination of two or more endogenous genes selected from kgtP, proV, ybjL and sspA; such as a combination selected from kgtP, proV and ybjL; kgtP and proV; kgtP and ybjL and kgtP and sspA, and/or (c) express a mutant of at least one of SpoT, PolB, RpoC, RpoB, Rnt and SapC and/or increase the expression of PyrE; wherein steps (a), (b) and (c) can be performed in any order.

Also provided is a process for improving the tolerance of a bacterial cell to an aliphatic dicarboxylic acid comprising genetically modifying the bacterial cell to (a) knock-down or knock-out at least one endogenous gene selected from the group consisting of kgtP, ybjL, proV, proW, proX, proQ, cspE, rfaE, yfbP, yfjM, pstS, pstA, pstB, pstC, rph, rpoS, sspA, tdk, uvrB, ycjG, and yeaR; such as a combination of two or more endogenous genes selected from kgtP, proV, ybjL and sspA; such as a combination selected from kgtP, proV and ybjL; kgtP and proV; kgtP and ybjL and kgtP and sspA; and/or (b) express a mutant of at least one of SpoT, PolB, RpoC, RpoB, Rnt and SapC and/or increase the expression of PyrE, wherein steps (a) and (b) are performed in any order.

The bacterial cell may, for example, be derived from the Escherichia, Lactobacillus, Lactococcus, Bacillus, Pseudomonas, Corynebacterium, Deinococcus or Ralstonia species, such as the Escherichia coli species.

Also provided is a method for producing an aliphatic dicarboxylic acid, comprising culturing such genetically modified bacterial cells in the presence of a carbon source, and, optionally, isolating the aliphatic dicarboxylic acid.

Also provided is a composition comprising glutaric acid or adipic acid at a concentration of at least 5 g/L and a plurality of bacterial cells of the Escherichia genus genetically modified to (a) knock-down or knock-out at least one endogenous gene selected from the group consisting of kgtP, ybjL, proV, proW, proX, proQ, cspE, rfaE, yfbP, yfjM, pstS, pstA, pstB, pstC, rph, rpoS, sspA, tdk, uvrB, ycjG, and yeaR; such as a combination of two or more endogenous genes selected from kgtP, proV, ybjL and sspA; such as a combination selected from kgtP, proV and ybjL; kgtP and proV; kgtP and ybjL and kgtP and sspA; and/or (b) express a mutant of at least one of SpoT, PolB, RpoC, RpoB, Rnt and SapC and/or increase expression of PyrE.

In embodiments where the bacterial cell comprises a biosynthetic pathway for producing an aliphatic dicarboxylic acid, the pathway may, for example, comprise
(a) a lysine monooxygenase, a 5-aminovaleramidase, a 5-aminovalerate transaminase, and a glutaraldehyde semialdehyde dehydrogenase;
(b) a reversible 3-oxoadipyl-CoA thiolase, a 3-hydroxyacyl-CoA dehydrogenase, an enoyl-CoA hydratase, an enoyl-CoA reductase, and either a terminal carboxyacyl-CoA thioesterase, or a terminal carboxyacyl-CoA phosphotransferase and a reversible alkyl-1,n-dicarboxylate kinase, where n is the carbon chain length of the product; and, optionally, a malonyl-CoA or glutaryl-CoA transferase; or
(c) a 2-dehydro-3-deoxy-D-arabinoheptonate-7-phosphate synthase, a 3-dehydroquinate synthase, a 3-dehydroxyquinate dehydratase, a dehydroshikimic acid dehydratase, a protocatechuate decarboxylase, and a catechol 1,2-dioxygenase.

### Definitions

Unless otherwise indicated or contradicted by context, a “diacid” as used herein is an aliphatic dicarboxylic acid of the general formula COOH—R—COOH (I), where R is an alkyl chain. An “aliphatic diacid” or “aliphatic dicarboxylic acid” herein refers to an organic compound comprising an aliphatic carbon chain to which two or more carboxyl (—COOH) groups are attached, and includes linear aliphatic diacids, as well as derivatives thereof. Aliphatic diacids suitable for production in bacteria typically comprise from 3 to 12 carbon atoms, preferably 3 to 10 carbon atoms, more preferably 3 to 8 carbon atoms, even more preferably, 4 to 7 or 5 to 8 carbon atoms, and, most preferably, 5 to 7 carbon atoms, and optionally comprises one or more heteroatoms or other substituents. Examples of heteroatoms include oxygen (e.g., in the form of an oxo group, a.k.a. keto group), nitrogen, sulphur and halogens. Examples of other substituents include hydroxyl groups, amino groups, carboxyl groups, and alkyl groups. Preferred aliphatic diacids include, but are not limited to, glutaric acid, adipic acid, succinic acid, muconic acid, fumaric acid, itaconic acid, malic acid, malonic acid, maleic acid, glucaric acid, pimelic acid, suberic acid, sebacic acid, 2,5-furandicarboxylic acid, terephthalic acid, or azelaic acid, mesaconic acid, citraconic acid, tartaric acid, tartronic acid, diaminopimelic acid and glutaconic acid. In some embodiments, the aliphatic diacid does not comprise any heteroatom substituents. In some embodiments, the aliphatic diacid does not comprise any substituents. Glutaric, adipic, pimelic and sebacic acid are particularly preferred.

As used herein, a “recombinant biosynthetic pathway” for a compound of interest refers to an enzymatic pathway resulting in the production of a compound of interest in a host cell, wherein at least one of the enzymes is expressed from a transgene, i.e., a gene added to the host cell genome by transformation. In some cases, the recombinant biosynthetic pathway also comprises a deletion of one or more native genes in the host cell. The compound of interest is typically a diacid, and may be the actual end product or a precursor or intermediate in the production of another end product.

The terms “tolerant” or “improved tolerance”, when used to describe a genetically modified bacterial cell of the invention or a strain derived therefrom, refers to a genetically modified bacterial cell or strain that shows a reduced lag time, an improved growth rate, or both, in the presence of a diacid than the parent bacterial cell or strain from which it is derived, typically at concentrations of 1 g/L, such as at least 2 g/L or higher, such as at least 5 g/L or higher, such as at least 10 g/L or higher, such as at least 20 g/L or higher, such as at least 40 g/L or higher, such as at least 75 g/L or higher, such as at least 100 g/L or higher. An improved growth rate is at least 5%, such as at least 10%, such as at least 20%, such as at least 50%, such as at least 75% higher than that of a control, typically the parent cell or strain. A reduced lag time is at least 10%, such as at least 20%, such as at least 50%, such as at least 75%, such as at least 90% shorter than that of a control, typically the parent cell or strain.

The term “gene” refers to a nucleic acid sequence that encodes a cellular function, such as a protein, optionally including regulatory sequences preceding (5′ non-coding sequences) and following (3′ non-coding sequences) the coding sequence. An “endogenous gene” refers to a native gene in its natural location in the genome of an organism. A “transgene” is a gene, native or heterologous, that has been introduced into the genome by a transformation procedure. Genes names are herein set forth in italicised text with a lower-case first letter (e.g., metJ) whereas protein names are set forth in normal text with a capital first letter (e.g., MetJ).

As used herein the term “coding sequence” refers to a DNA sequence that encodes a specific amino acid sequence.

The term “native”, when used to characterize a gene or a protein herein with respect to a host cell, refers to a gene or protein having the nucleic acid or amino acid sequence as found in the host cell.

The term “heterologous”, when used to characterize a gene or protein with respect to a host cell, refers to a gene or protein which has a nucleic acid or amino acid sequence not normally found in the host cell.

As used herein the term “transformation” refers to the transfer of a nucleic acid fragment, such as a gene, into a host cell. Host cells containing a gene introduced by transformation or a “transgene” are referred to as “transgenic” or “recombinant” or “transformed” cells.

As used herein, a “genetic modification” or “genetically modified” refers to the introduction a genetically inherited change in the host cell genome. Examples of changes include mutations in genes and regulatory sequences, coding and non-coding DNA sequences. “Mutations” include deletions, substitutions and insertions of one or more nucleotides or nucleic acid sequences in the genome. Other genetic modifications include the introduction of heterologous genes or coding DNA sequences on a plasmid and/or into a chromosome by recombinant techniques. In one embodiment, the genetic modification is in a chromosome.

The term “expression”, as used herein, refers to the process in which a gene is transcribed into mRNA, and may optionally include the subsequent translation of the mRNA into an amino acid sequence, i.e., a protein or polypeptide.

As used herein, “reduced expression” or “downregulation” of an endogenous gene in a host cell means that the levels of the mRNA, protein and/or protein activity encoded by the gene are significantly reduced in the host cell, typically by at least 25%, such as at least 50%, such as at least 75%, such as at least 90%, such as at least 95%, as compared to a control. Typically, when the reduced expression is obtained by a genetic modification in the host cell, the control is the unmodified host cell. Sometimes, e.g., in the case of gene knock-out, the reduction of native rnRNA and functional protein encoded by the gene is higher, such as 99% or greater.

“Increased expression”, “upregulation”, “overexpressing” or the like, when used in the context of a protein or activity described herein, means increasing the protein level or activity within a bacterial cell. An up-regulation of an activity can occur through, e.g., increased activity of a protein, increased potency of a protein or increased expression of a protein. The protein with increased activity, potency or expression can be encoded by genes disclosed herein.

Genetic modifications resulting in a reduced expression of a target gene/protein can include, e.g., knock-down of the gene (e.g., a mutation in a promoter or other expression control sequence that results in decreased gene expression), a knock-out or disruption of the gene (e.g., a mutation or deletion of the gene that results in 99 percent or greater decrease in gene expression), a mutation or deletion in the coding sequence which results in the expression of non-functional protein, and/or the introduction of a nucleic acid sequence that reduces the expression of the target gene, e.g. a repressor that inhibits expression of the target or inhibitory nucleic acids (e.g. CRISPR/dCas9, antisense RNA, etc.) that reduces the expression of the target gene.

Standard recombinant DNA and molecular cloning techniques used here are well known in the art and are described by Sambrook, J., Fritsch, E. F. and Maniatis, T. Molecular Cloning: A Laboratory Manual, 4^{th} ed.; Cold Spring Harbor Laboratory: Cold Spring Harbor, N.Y., 2012; and by Silhavy, T. J., Berman, M. L. and Enquist, L. W. Experiments with Gene Fusions; Cold Spring Harbor Laboratory: Cold Spring Harbor, N.Y., 1984; and by Ausubel, F. M. et al., In Current Protocols in Molecular Biology, published by John Wiley & Sons (1995); and by Datsenko and Wanner, 2000; and by Baba et al., 2006; and by Thomason et al., 2007.

A “conservative” amino acid substitution in a protein is one that does not negatively influence protein activity. Typically, a conservative substitution can be made within groups of amino acids sharing physicochemical properties, such as, e.g., basic amino acids (arginine, lysine and histidine), acidic amino acids (glutamic acid and aspartic acid), polar amino acids (glutamine and asparagines), hydrophobic amino acids (leucine, isoleucine, valine and methionine), aromatic amino acids (phenylalanine, tryptophan and tyrosine), and small amino acids (glycine, alanine, serine, and threonine). Most commonly, substitutions can be made between Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, Asp/Gly. Other preferred substitutions are set out in Table 1 below.

TABLE 1
Examples of amino acid substitutions.

| Original | | Preferred |
|---|---|---|
| amino acid | Examples of substitutions | substitution |
| Ala (A) | val; leu; ile | Val |
| Arg (R) | lys; gln; asn | Lys |
| Asn (N) | gln; his; asp, lys; arg | Gln |
| Asp (D) | glu; asn | Glu |
| Cys (C) | ser; ala | Ser |
| Gln (Q) | asn; glu | Asn |
| Glu (E) | asp; gln | Asp |
| Gly (G) | Ala | Ala |
| His (H) | asn; gln; lys; arg | Arg |
| Ile (I) | leu; val; met; ala; phe; norleucine | Leu |
| Leu (L) | norleucine; ile; val; met; ala; phe | Ile |
| Lys (K) | arg; gln; asn | Arg |
| Met (M) | leu; phe; ile | Leu |
| Phe (F) | leu; val; ile; ala; tyr | Tyr |
| Pro (P) | Ala | Ala |
| Ser (S) | thr | Thr |
| Thr (T) | Ser | Ser |
| Trp (W) | tyr; phe | Tyr |
| Tyr (Y) | trp; phe; thr; ser | Phe |
| Val (V) | ile; leu; met; phe; ala; norleucine | Leu |

### Specific Embodiments of the Invention

As described herein, the invention provides bacterial cells with improved tolerance to one or more diacids, as well as related processes and materials for producing and using such bacterial cells.

### 1) Genetic Modifications

The genetic modifications according to the invention include those resulting in reduced expression of genes, e.g., by gene knock-down or knock-out, herein referred to as “Group 1 modifications”; as well as silent mutations in coding or non-coding regions and non-silent (i.e., coding) mutations in coding regions, herein referred to as “Group 2 modifications”; and combinations thereof.

In a preferred embodiment, the one or more genetic modifications provide for an increased growth rate, a reduced lag time, or both, of the bacterial cell in the presence of at least one of glutaric and adipic acid as compared to the bacterial cell without the genetic modification, e.g., the parent or wild-type bacterial cell. The glutaric and/or adipic acid may be present in the growth medium at, e.g., a concentration of at least about 1 g/L, such as at least about 2 g/L, such as at least about 5 g/L, such as at least about 10 g/L, such as at least about 20 g/L.

a) Group 1 Modifications

In one aspect, the bacterial cell has at least one genetic modification which reduces expression an endogenous gene selected from the group consisting of kgtP, ybjL, proV, proW, proX, proQ, cspE, rfaE, yfbP, yfjM, pstS, pstA, pstB, pstC, rph, rpoS, sspA, tdk, uvrB, ycjG, and yeaR.

For example, in one embodiment, the expression of one or more of kgtP, ybjL, proV, proW, proX, proQ and sspA, such as kgtP, ybjL, proV or sspA, is reduced. In one specific embodiment, the expression of kgtP is reduced, optionally wherein the expression of lysP is not reduced. In one specific embodiment, the expression of ybjL is reduced. In one specific embodiment, the expression of sspA is reduced. In one specific embodiment, the expression of proV, proW, proX or proQ, such as e.g. proV, is reduced, optionally wherein the expression of marR is not reduced. In another specific embodiment, the expression of cspE, rfaE, yfbP, yfjM, pstS, pstA, pstB, pstC, rph, rpoS, sspA, tdk, uvrB, ycjG, or yeaR is reduced.

In another aspect, there is provided a bacterial cell which comprises genetic modifications reducing the expression of two or more endogenous genes, wherein at least one gene is selected from the group consisting of kgtP, ybjL, proV, proW, proX, proQ, cspE, rfaE, yfbP, yfjM, pstS, pstA, pstB, pstC, rph, rpoS, sspA, tdk, uvrB, ycjG, and yeaR. In one embodiment, the bacterial cell comprises a genetic modification reducing the expression of kgtP but no genetic modification which reduces the expression of lysP. In one embodiment, the bacterial cell comprises a genetic modification reducing the expression of proV but no genetic modification which reduces the expression of marR. In one embodiment, the bacterial cell comprises genetic modifications which reduce the expression of at least two genes selected from the group consisting of kgtP, ybjL, proV, proW, proX, proQ, cspE, rfaE, yfbP, yfjM, pstS, pstA, pstB, pstC, rph, rpoS, sspA, tdk, uvrB, ycjG, and yeaR.

In one embodiment, the genetic modifications reduce the expression of kgtP and one or more other endogenous genes, optionally selected from ybjL, proV, proW, proX, proQ, cspE, rfaE, yfbP, yfjM, pstS, pstA, pstB, pstC, rph, rpoS, sspA, tdk, uvrB, ycjG and yeaR, such as from ybjL, proV, proW, proX, and sspA, optionally wherein the other endogenous genes do not comprise lysP. In separate and specific embodiments, the bacterial cell comprises genetic modifications which reduce the expression of kgtP and ybjL, kgtP and proV, proV and ybjL, or kgtP and sspA.

In one embodiment, the genetic modifications reduce the expression of kgtP and two or more endogenous genes selected from ybjL, proV, proW, proX, proQ, cspE, rfaE, yfbP, yfjM, pstS, pstA, pstB, pstC, rph, rpoS, sspA, tdk, uvrB, ycjG and yeaR, wherein at least one endogenous gene is selected from ybjL, proV, proW, proX, and sspA. In one specific embodiment, the bacterial cell comprises genetic modifications which reduce the expression of kgtP, proV and ybjL.

In other separate and specific embodiments, the bacterial cell comprises:
a first genetic modification which reduces the expression of ybjL, and a second genetic modification which reduces the expression of a gene selected from kgtP, proV, proW, proX, proQ, cspE, rfaE, yfbP, yfjM, pstS, pstA, pstB, pstC rph, rpoS, sspA, tdk, uvrB, ycjG, and yeaR;
a first genetic modification which reduces the expression of proV and a second genetic modification which reduces the expression of a gene selected from of kgtP, ybjL, proW, proX, proQ, cspE, rfaE, yfbP, yfjM, pstS, pstA, pstB, pstC, rph, rpoS, sspA, tdk, uvrB, ycjG, and yeaR;
a first genetic modification which reduces the expression of prow and a second genetic modification which reduces the expression of a gene selected from of kgtP, ybjL, proV, proX, proQ, cspE, rfaE, yfbP, yfjM, pstS, pstA, pstB, pstC, rph, rpoS, sspA, tdk, uvrB, ycjG, and yeaR;
a first genetic modification which reduces the expression of proX and a second genetic modification which reduces the expression of a gene selected from of kgtP, ybjL, proV, proW, proQ, cspE, rfaE, yfbP, yfjM, pstS, pstA, pstB, pstC, rph, rpoS, sspA, tdk, uvrB, ycjG, and yeaR;
a first genetic modification which reduces the expression of proQ and a second genetic modification which reduces the expression of a gene selected from of kgtP, ybjL, proV, proW, proX, cspE, rfaE, yfbP, yfjM, pstS, pstA, pstB, pstC, rph, rpoS, sspA, tdk, uvrB, ycjG, and yeaR;
a first genetic modification which reduces the expression of cspE and a second genetic modification which reduces the expression of a gene selected from of kgtP, ybjL, proV, proW, proX, proQ, rfaE, yfbP, yfjM, pstS, pstA, pstB, pstC, rph, rpoS, sspA, tdk, uvrB, ycjG, and yeaR;
a first genetic modification which reduces the expression of rfaE and a second genetic modification which reduces the expression of a gene selected from of kgtP, ybjL, proV, proW, proX, proQ, cspE, yfbP, yfjM, pstS, pstA, pstB, pstC, rph, rpoS, sspA, tdk, uvrB, ycjG, and yeaR;
a first genetic modification which reduces the expression of yfbP and a second genetic modification which reduces the expression of a gene selected from of kgtP, ybjL, proV, proW, proX, proQ, cspE, rfaE, yfjM, pstS, pstA, pstB, pstC, rph, rpoS, sspA, tdk, uvrB, ycjG, and yeaR;
a first genetic modification which reduces the expression of yfjM and a second genetic modification which reduces the expression of a gene selected from of kgtP, ybjL, proV, proW, proX, proQ, cspE, rfaE, yfbP, pstS, pstA, pstB, pstC, rph, rpoS, sspA, tdk, uvrB, ycjG, and yeaR;
a first genetic modification which reduces the expression of pstS and a second genetic modification which reduces the expression of a gene selected from of kgtP, ybjL, proV, proW, proX, proQ cspE, rfaE, yfbP, yfjM, rph, rpoS, sspA, tdk, uvrB, ycjG, and yeaR;
a first genetic modification which reduces the expression of pstA and a second genetic modification which reduces the expression of a gene selected from of kgtP, ybjL, proV, proW, proX, proQ, cspE, rfaE, yfbP, yfjM, rph, rpoS, sspA, tdk, uvrB, ycjG, and yeaR;
a first genetic modification which reduces the expression of pstB and a second genetic modification which reduces the expression of a gene selected from of kgtP, ybjL, proV, proW, proX, proQ, cspE, rfaE, yfbP, yfjM, rph, rpoS, sspA, tdk, uvrB, ycjG, and yeaR;
a first genetic modification which reduces the expression of pstC and a second genetic modification which reduces the expression of a gene selected from of kgtP, ybjL, proV, proW, proX, proQ, cspE, rfaE, yfbP, yfjM, rph, rpoS, sspA, tdk, uvrB, ycjG, and yeaR;
a first genetic modification which reduces the expression of rph and a second genetic modification which reduces the expression of a gene selected from of kgtP, ybjL, proV, proW, proX, proQ cspE, rfaE, yfbP, yfjM, pstS, pstA, pstB, pstC, rpoS, sspA, tdk, uvrB, ycjG, and yeaR;
a first genetic modification which reduces the expression of rpoS and a second genetic modification which reduces the expression of a gene selected from of kgtP, ybjL, proV, proW, proX, proQ cspE, rfaE, yfbP, yfjM, pstS, pstA, pstB, pstC, rph, sspA, tdk, uvrB, ycjG, and yeaR;
a first genetic modification which reduces the expression of sspA and a second genetic modification which reduces the expression of a gene selected from of kgtP, ybjL, proV, proW, proX, proQ, cspE, rfaE, yfbP, yfjM, pstS, pstA, pstB, pstC, rph, rpoS, tdk, uvrB, ycjG, and yeaR;
a first genetic modification which reduces the expression of tdk and a second genetic modification which reduces the expression of a gene selected from of kgtP, ybjL, proV, proW, proX, proQ, cspE, rfaE, yfbP, yfjM, pstS, pstA, pstB, pstC, rph, rpoS, sspA, uvrB, ycjG, and yeaR;
a first genetic modification which reduces the expression of uvrB and a second genetic modification which reduces the expression of a gene selected from of kgtP, ybjL, proV, proW, proX, proQ, cspE, rfaE, yfbP, yfjM, pstS, pstA, pstB, pstC, rph, rpoS, sspA, tdk, ycjG, and yeaR;
a first genetic modification which reduces the expression of ycjG and a second genetic modification which reduces the expression of a gene selected from of kgtP, ybjL, proV, proW, proX, proQ, cspE, rfaE, yfbP, yfjM, pstS, pstA, pstB, pstC, rph, rpoS, sspA, tdk, uvrB, and yeaR; or
a first genetic modification which reduces the expression of yeaR and a second genetic modification which reduces the expression of a gene selected from of kgtP, ybjL, proV, proW, proX, proQ, cspE, rfaE, yfbP, yfjM, pstS, pstA, pstB, pstC rph, rpoS, sspA, tdk, uvrB, and ycjG.

In one specific embodiment, either one or both of the first and second genetic modifications is a knock-out of the gene, optionally a deletion. In an alternative embodiment at least one of the first and second genetic modifications is a knock-down of the gene.

In one aspect, there is provided a bacterial cell according to any one of the preceding aspects and embodiments, wherein the genetic modification is a knock-down of the one or more endogenous genes, resulting in at least 25%, such as at least 50%, such as at least 75%, such as at least 90%, such as at least 95%, reduction in the level of mRNA encoded by the gene.

In one aspect, there is provided a bacterial cell according to any one of the preceding aspects and embodiments, wherein the genetic modification is a knock-down of the one or more endogenous genes, resulting in at least 25%, such as at least 50%, such as at least 75%, such as at least 90%, such as at least 95%, reduction in the level of protein encoded by the gene.

In one aspect, there is provided a bacterial cell according to any one of the preceding aspects and embodiments, wherein the genetic modification is a knock-out of the one or more endogenous genes.

Knock-down or knock-out of a gene can be accomplished by any method known in the art for bacterial cells, and include, e.g., lambda Red mediated recombination, P1 phage transduction, and single-stranded oligonucleotide recombineering/MAGE technologies (see, e.g., Datsenko and Wanner, 2000; Thomason et al., 2007a,b; Wang et al., 2009). Typically, a knock-down of a gene can be accomplished by, for example, a mutation in the promoter region resulting in decreased transcription, a deletion or mutation in the coding region of the gene resulting in a reduced or fully or substantially eliminated activity of the protein, or by the presence of antisense sequences that interfere with transcription or translation of the gene, resulting in reduced expression of the protein. Preferably, the knocking-down of a gene results in at least 20% reduction in the expression level of the gene product in the bacterial cell, such as at least 30%, such as at least 40%, such as at least 50%, such as at least 60%, such as at least 70%, such as at least 80%, such as at least 90%, such as at least 95% or higher.

A knock-out of a gene includes elimination of a gene's expression, such as by introducing a mutation in the coding sequence and/or promoter so that at least a portion (up to and including all) of the coding sequence and/or promoter is disrupted, shifted or deleted, resulting in loss of expression of the protein, or expression only of a non-functional mutant or non-functional fragment of the endogenous protein. As used herein, the symbol “A”, i.e., the greek uppercase letter for “delta”, denotes a deletion of an endogenous gene. Preferably, a knock-out of a gene results in 1% or less of the native gene product being detectable, such as no detectable gene product.

b) Group 2 Modifications

In certain embodiments, a mutant protein is expressed in the bacterial cell, e.g., from a mutated version of an endogenous gene, or from a transgene encoding the mutant protein.

In one aspect, the bacterial cell comprises a Group 2 modification, e.g., a mutation in one or more of SpoT, PolB, RpoC, RpoB, Rnt and SapC; an increased expression of one or more of SpoT, PolB, RpoC, RpoB, Rnt and SapC; and/or a mutation in rph or the pyrE/rph intergenic region which increases the expression of pyrE, wherein the one or more mutations improve tolerance to at least one aliphatic diacid such as, e.g. glutaric or adipic acid. Preferably, the bacterial cell further comprises a Group 1 modification according to any aspect or embodiment herein.

In one embodiment, the Group 2 modification comprises a mutant SpoT, comprising one or more mutations. The mutations may be located, for example, in the threonyl-tRNA synthetase GTPase and SpoT (TGS) domain corresponding to amino acid residues 1388 to T447, or the linker region between the TGS domain and the aspartokinase, chorismate mutase and TyrA (ACT) domain corresponding to A448 to T621 in E. coli SpoT. Without being limited to theory, since the mutations identified are near the TGS domain which is involved in nucleotide binding, at least some of the mutations may decrease the ppGpp synthetase activity of SpoT, e.g., by decreasing its binding affinity to substrates such as ATP or (p)ppGpp, or increasing its binding affinity to products such as GTP, AMP, or GDP. This may, in turn, reduce sensitivity of the cells to accumulating ppGpp and delay the onset of the stringent response. The stringent response may be activated under general stress conditions such as in high concentrations of diacids and might prevent growth under such conditions. In one embodiment, the SpoT mutant comprises a mutation in one or more amino acid residues selected from those corresponding to R236, M247, V422, S434, T442, A451, N454, W457, D580, N601, 1602 and R603 in E. coli SpoT. In one embodiment, the mutant SpoT comprises at least one amino acid substitution selected from V422A, A451D, A451V, W457C, N454H, D580Y, R236L, R236S, M247K, NIR(601-603)S, T442I, and S434L or a conservative substitution of any thereof. In a specific embodiment, the mutant SpoT comprises a mutation in A451 or R236, e.g., an amino acid substitution selected from A451D, A451V, R236L and R236S, or a conservative substitution thereof, e.g., selected from A451E, A451N, A451G, A451A, A451L, A451I, R236I, R236V, R236T, R236A, R236N and R236G.

In one embodiment, the Group 2 modification comprises a mutant PolB comprising one or more mutations. The mutation may be located, e.g., in the residue corresponding to R477 in E. coli PolB, and may be an amino acid substitution such as R477G or a conservative substitution thereof, e.g., R477A, R477D or R477S.

In one embodiment, the Group 2 modification comprises a mutant RpoC comprising one or more mutations. The mutation may be located in, e.g., the residue corresponding to H419 and/or P64 in E. coli RpoC, and may be an amino acid substitution such as H419P, P64L, or a conservative substitution thereof, e.g., H419A, P64I, P64V, P64M, P64A or P64F. Without being limited to theory, since some of these residues (e.g., H419) are close to residues involved in ppGpp-binding, at least some of them may decrease ppGpp binding to RpoC which in turn may reduce sensitivity of the cells to accumulating ppGpp and delay the onset of the stringent response.

In one embodiment, the Group 2 modification comprises a mutant RpoB comprising one or more mutations. The mutation may be located in, e.g., the residue corresponding to K203 in E. coli RpoB, and may be an amino acid substitution such as K203T or a conservative substitution thereof, e.g., K203S or K203A. Without being limited to theory, since residue K203 is near the entrance for dsDNA, it may interact with phosphate on dsDNA, possibly reducing premature transcription termination.

In one embodiment, the Group 2 modification comprises a mutant Rnt comprising one or more mutations. The mutations may be located, for example, in or close to catalytic residues of conserved exonuclease motifs corresponding to positions 23, 25, 181, and 186 in E. coli Rnt. In one embodiment, the Rnt mutant comprises a mutation in one or more amino acid residues selected from those corresponding to Q179, A27, F194 and A180. In one embodiment, the mutant Rnt comprises at least one amino acid substitution selected from Q179P, A27T, F194L and A180T or a conservative substitution of any thereof, e.g., Q179A, A27S, A27G, F1951, F195V, F195T, F195A, A180S and A180G.

In one embodiment, the Group 2 modification comprises a mutant SapC comprising one or more mutations. The mutation may be located in, e.g., the residue corresponding to G79 in E. coli SapC, and may be an amino acid substitution such as G79W or a conservative substitution thereof, e.g., G79Y or G79F.

In one embodiment, the bacterial cell comprises one or more mutations which increase(s) the expression level or activity of PyrE, optionally in combination with a Group 1 modification. E. coli K-12 MG1655 and W3110, plus their common ancestor strain W1485, are known to exhibit pyrimidine starvation in minimal media due to the presence a frameshift mutation occurring in rph relative to other E. coli strains (Jensen et al., 1993). This mutation disrupts the transcriptional/translational coupling required for efficient translation of pyrE, encoding orotate phosphoribosyltransferase in the pyrimidine biosynthesis pathway. Compensatory mutations that correct this deficiency are well-known in the art. One of these mutations is an 82 bp deletion near the 3′ terminus of rph, due to presence of two homologous GCAGAAGGC sequences flanking this 82 bp region (Conrad et al., 2009). In addition to the 82 bp deletion, a 1 bp deletion at coordinate 3815809 in the pyrE/rph intergenic region has previously been encountered in strains evolved for growth on a minimal glucose medium (LaCroix et al., 2015), and a wide array of other frameshift mutations, substitutions, and coding mutations near the 3′ terminus of rph were encountered in a short-term selection/evolution of combinatorial mutant libraries in minimal medium at an elevated temperature of 42° C. (Sandberg et al., 2014). Without being limited to theory, all of these mutations can serve the same function of increasing expression of PyrE, with the selective pressure for these mutations being even stronger in minimal media with particular imposed stresses (certain chemicals or heat) than in minimal media alone. In one embodiment, the bacterial cell comprises mutations in rph or the pyrE/rph intergenic region, such as, e.g., the 82 bp deletion near the 3′ terminus of rph, the 1 bp deletion in the intergenic region between pyrE and rph, or both. In one embodiment, increased expression of PyrE is achieved by transforming the bacterial cell with a transgene expressing the endogenous protein. Increased expression may be obtained by causing an up-regulation through increased expression of a protein, the copy number of a gene or genes encoding the protein may be increased. Alternatively, a strong and/or inducible promoter can be used to direct the expression of the gene, the gene being expressed either as a transient expression vehicle or homologously or heterologously incorporated into the bacterial genome. In another embodiment, the promoter, regulatory region and/or the ribosome binding site upstream of the gene can be altered to achieve the over-expression. The expression can also be enhanced by increasing the relative half-life of the messenger or other forms of RNA. Any one or a combination of these approaches can be used to effect upregulation of a desired target protein as needed.

In a specific embodiment, the bacterial cell comprises at least one Group 1 modification and at least one Group 2 modification. Non-limiting examples of Group 1 modifications for combination with any one or more of the overexpressed or mutant SpoT, PolB, RpoC, RpoB, Rnt, SapC and pyrE/rph include knock-down or knock-out of
i) at least one endogenous gene selected from the group consisting of kgtP, ybjL, proV, proW, proX, proQ, cspE, rfaE, yfbP, yfjM, pstS, pstA, pstB, pstC, rph, rpoS, sspA, tdk, uvrB, ycjG, and yeaR; and
ii) a combination of two or more endogenous genes selected from kgtP, proV, ybjL and sspA, such as kgtP, proV and ybjL; kgtP and proV; kgtP and ybjL; and kgtP and sspA.

In separate and specific embodiments, the bacterial cell comprises:
a mutation selected from SpoT-V422A, SpoT-A451D, SpoT-A451V, SpoT-W457C, SpoT-N454H, SpoT-D580Y, SpoT-R236L, SpoT-R236S, SpoT-M247K, SpoT-NIR(601-603)S, SpoT-T442I, SpoT-S434L, PolB-R477G, RpoC-H419P, RpoC-P64L, RpoB-K203T, Rnt-Q179P, Rnt-A27T, Rnt-F194L, Rnt-A180T, and SapC-G79W
a mutation selected from SpoT-V422A, SpoT-A451D, SpoT-A451V, SpoT-W457C, SpoT-N454H, SpoT-D580Y, SpoT-R236L, SpoT-R236S, SpoT-M247K, SpoT-NIR(601-603)S, SpoT-T442I, SpoT-S434L, PolB-R477G, RpoC-H419P, RpoC-P64L, RpoB-K203T, Rnt-Q179P, Rnt-A27T, Rnt-F194L, Rnt-A180T, and SapC-G79W, and a knock-out or knockdown of at least one of kgtP, ybjL, proV, proW, proX, proQ, cspE, rfaE, yfbP, yfjM, pstS, pstA, pstB, pstC, rph, rpoS, sspA, tdk, uvrB, ycjG, or yeaR, such as kgtP.
a mutation selected from SpoT-V422A, SpoT-A451D, SpoT-A451V, SpoT-W457C, SpoT-N454H, SpoT-D580Y, SpoT-R236L, SpoT-R236S, SpoT-M247K, SpoT-NIR(601-603)S, SpoT-T442I, SpoT-S434L, PolB-R477G, RpoC-H419P, RpoC-P64L, RpoB-K203T, Rnt-Q179P, Rnt-A27T, Rnt-F194L, Rnt-A180T, and SapC-G79W, and a knock-out or knockdown of at least two of kgtP, ybjL, and proV (or genes encoding other subunits in the same protein complex, which are proX and proW), such as kgtP and proV.
a mutation selected from SpoT-V422A, SpoT-A451D, SpoT-A451V, SpoT-W457C, SpoT-N454H, SpoT-D580Y, SpoT-R236L, SpoT-R236S, SpoT-M247K, SpoT-NIR(601-603)S, SpoT-T442I, SpoT-S434L, PolB-R477G, RpoC-H419P, RpoC-P64L, RpoB-1K203T, Rnt-Q179P, Rnt-A27T, Rnt-F194L, Rnt-A180T, and SapC-G79W, and a knock-out or knockdown of kgtP, ybjL, and proV (or genes encoding other subunits in the same protein complex, which are proX and proW) in combination, such as kgtP, proV, and ybjL.
a mutation increasing the expression of pyrE and a knock-out or knockdown of kgtP, ybjL, proV, proW, proX, proQ, cspE, rfaE, yfbP, yfjM, pstS, pstA, pstB, pstC, rph, rpoS, sspA, tdk, uvrB, ycjG, or yeaR, such as kgtP.
a mutation increasing the expression of pyrE and a knock-out or knockdown of at least two of kgtP, ybjL, and proV (or genes encoding other subunits in the same protein complex, which are proX and proW), such as kgtP and proV.
a mutation increasing the expression of pyrE and a knock-out or knockdown of kgtP, ybjL, and proV (or genes encoding other subunits in the same protein complex, which are proX and proW) in combination, such as kgtP, proV, and ybjL.

In other separate and specific embodiments, the bacterial cell comprises:
a combination of mutations SpoT-V422A and RpoC-H419P, and a knock-out or knockdown of kgtP.
a mutation SpoT-V422A, and a knock-out or knockdown of kgtP and at least one of proV, proW and proX, and a knock-out, knockdown, or reduction of function mutation in sspA.
a mutation SpoT-A451D, and a knock-out or knockdown of kgtP.
a mutation SpoT-A451D, and a knock-out or knockdown of kgtP and at least one of proV, proW and proX.
a combination of mutations SpoT-A451D and RpoC-H419P, and a knock-out or knockdown of kgtP and at least one of proV, proW and proX.
a combination of mutations SpoT-W457C and RpoC-H419P, and a knock-out or knockdown of kgtP.
a mutation SpoT-D580Y, and a knock-out or knockdown of kgtP and at least one of proV, proW and proX.
a combination of mutations SpoT-R236L and RpoB-K203T, and a knock-out or knockdown of kgtP and sspA.
a combination of mutations PolB-R477G and RpoC-P64L, and a knock-out or knockdown of kgtP and at least one of proV, proW and proX.
a knock-out or knockdown of kgtP and ybjL, and a mutation that increases the expression of PyrE.
a knock-out or knockdown of kgtP and a mutation that increases the expression of PyrE.
a knock-out of knockdown of kgtP, ybjL, and at least one of nagA or nagC.
a mutation SpoT-S434L, and a knock-out of knockdown of kgtP; at least one of proV, proW and proX; ybjL; and at least one of nagC and nagA.
a combination of mutations SpoT-S434L and ProQ-R80C, and a knock-out of knockdown of kgtP; at least one of proV, proW and proX; ybjL, and at least one of nagC and nagA.
a mutation SpoT-S434L, and a knock-out or knockdown of kgtP; at least one of proV, proW and proX; ybjL; and at least one of nagC and nagA; and proQ.
a knock-out of knockdown of kgtP and one of pstS, tdk, and rpoS, or any combination thereof.
a knock-out of knockdown of kgtP; ybjL; at least one of proV, proW and proX; and sspA.
a knock-out of knockdown of kgtP, tdk, and pstS.
2) Production Pathways

In some aspects, the bacterial cell comprises a recombinant pathway for producing an aliphatic diacid of interest, optionally providing for a production level of at least about 5 g/L of the aliphatic diacid over a predetermined period of time, e.g., about 200h, about 100h, about 72h, about 48h or about 24h. A recombinant pathway can, for example, be added to introduce the capability to produce the diacid in a bacterial cell which does not have a native pathway to do so, typically by transforming the cell with one or more heterologous enzymes catalyzing the desired reaction(s). Alternatively, in cases where the bacterial cell has a native pathway for production of the diacid of interest, a recombinant pathway can nonetheless be introduced in order to increase the production yield, e.g., by overexpressing one or more native enzymes or transforming the cell with heterologous enzymes. In separate and specific embodiments, the recombinant pathway provides for a production level of at least 5 g/L, such as at least 10 g/L or higher, such as at least 20 g/L or higher, such as at least 40 g/L or higher, such as at least 45 g/L or higher, such as at least 75 g/L or higher, such as at least 100 g/L or higher.

So, in one aspect, there is provided a bacterial cell with improved tolerance to at least one aliphatic diacid according to any aspect or embodiment described herein, wherein the bacterial cell further comprises a recombinant biosynthetic pathway for producing an aliphatic diacid of interest, such as, e.g., glutaric acid, adipic acid, succinic acid, muconic acid, fumaric acid, itaconic acid, malic acid, malonic acid, maleic acid, glucaric acid, pimelic acid, suberic acid, sebacic acid, 2,5-furandicarboxylic acid, terephthalic acid, or azelaic acid, mesaconic acid, citraconic acid, tartaric acid, tartronic acid, diaminopimelic acid or glutaconic acid. In principle, any such recombinant biosynthetic pathway which is known in the art can be introduced into the cell by standard recombinant technologies. Some specific, preferred pathways are, however, exemplified below and in Example 1—see the section entitled “Biological production of diacids” and references cited therein.

It is to be understood that, when a specific enzyme of these biosynthetic pathways is mentioned by name such as, e.g., “lysine monooxygenase”, the enzyme may be any characterized and sequenced enzyme, from any species, that have been reported in the literature so long as it provides the desired activity. In some embodiments, the enzyme is an overexpressed gene which is native to the host cell used. In some embodiments, the enzyme is a functionally active fragment or variant of an enzyme which is heterologous or native to the host cell. Also, in some embodiments, the recombinant biosynthetic pathway comprises a knock-down or a knock-out of one or more genes, typically for the purpose of avoiding competing reactions reducing the yield of the desired aliphatic diacid.

So, in one embodiment, the biosynthetic pathway is for producing glutaric acid from glucose, and comprises genes, optionally overexpressed and/or heterologous, encoding:
a lysine monooxygenase;
a 5-aminovaleramidase;
a 5-aminovalerate transaminase, and
a glutarate semialdehyde dehydrogenase.

The bacterial cell may further be modified by one or more of
(i) introducing feedback resistance mutations in native genes corresponding to DapA (4-hydroxytetrahydrodipicolinate synthase) and LysC (asparate kinase III), optionally also overexpressing the modified proteins; and
(ii) knockdown or knock-out of native genes corresponding to cadA and/dcC;
(iii) constitutive overexpression of lysine biosynthesis, e.g., via a dapA promoter replacement; and
(iv) knock-down or knock-out of native genes corresponding to speE, speG, patA and puuPA.

In one embodiment, the biosynthetic pathway is for producing adipic acid and comprises
a reversible 3-oxoadipyl-CoA thiolase (e.g., PaaJ from E. coli),
a 3-hydroxyacyl-CoA dehydrogenase (e.g., PaaH1 from Ralstonia eutropha),
an enoyl-CoA hydratase (e.g., h16_AA307 gene product from Ralstonia eutropha H16),
a trans-enoyl-CoA reductase (e.g., Ter from Euglena gracilis),
a phosphate butyryltransferase (e.g., Ptb from Clostridium acetobutylicum), and
a butyryl kinase (e.g., Buk1 from Clostridium acetobutylicum)

The bacterial cell may further be modified to knock-down or knock-out one or more native genes corresponding to ptsG, poxB, pta, sdhA, and iclR.

In one embodiment, the biosynthetic pathway is for producing adipic acid and comprises
a 3-oxoadipyl-CoA thiolase (e.g., PaaJ from E. coli),
a 3-hydroxyacyl-CoA dehydrogenase (e.g. PaaH from E. coli)
an enoyl-CoA hydratase (e.g. PaaF from E. coli)
an enoyl-CoA reductase (e.g. Ter from Treponema denticola)
an acyl-CoA thioesterase (e.g. Acot8 from Mus musculus)

The bacterial cell may further be modified to knock-down or knock-out one or more native genes corresponding to pta, poxB, ldhA, and adhE.

In one embodiment, the biosynthetic pathway is for producing pimelic acid and comprises feeding glutaric acid and
a glutaryl-CoA transferase (e.g., Cat1 from Clostridium kluyveri),
a 3-oxoadipyl-CoA thiolase (e.g., PaaJ from E. coli),
a 3-hydroxyacyl-CoA dehydrogenase (e.g. PaaH from E. coli)
an enoyl-CoA hydratase (e.g. PaaF from E. coli)
an enoyl-CoA reductase (e.g. Ter from Treponema denticola)
an acyl-CoA thioesterase (e.g. Acot8 from Mus musculus)

The bacterial cell may further be modified to knock-down or knock-out one or more native genes corresponding to pta, poxB, IdhA, adhE, and frdA.

In one embodiment, the biosynthetic pathway is for producing sebacic acid and comprises
a glutaryl-CoA transferase (e.g., Cat1 from Clostridium kluyveri),
a 3-oxoadipyl-CoA thiolase (e.g., DcaF from Acinetobacter sp. ADP1),
a 3-hydroxyacyl-CoA dehydrogenase (e.g. DcaH from Acinetobacter sp. ADP1)
an enoyl-CoA hydratase (e.g. DcaE from Acinetobacter sp. ADP1)
an enoyl-CoA reductase (e.g. Ter from Treponema denticola)
an acyl-CoA thioesterase (e.g. Acot8 from Mus musculus)

The bacterial cell may further be modified to knock-down or knock-out one or more native genes corresponding to pta, poxB, ldhA, adhE, frdA, and native acyl-CoA thioesterases including yciA, ybgC, ydiI, tesA, fadM, and tesB.

In one embodiment, the biosynthetic pathway is for producing adipic acid and comprises
a 3-oxoadipyl-CoA thiolase/p-ketothioase (e.g. from Thermobifida fusca B6),
a 3-hydroxyacyl-CoA dehydrogenase (e.g. from Thermobifida fusca B6)
an enoyl-CoA hydratase (e.g. from Thermobifida fusca B6)
an enoyl-CoA reductase (e.g. from Thermobifida fusca B6)
a succinyl-CoA synthetase (e.g. Tfu_2577 and Tfu_2576 from Thermobifida fusca B6)

The bacterial cell may further be modified to knock-down or knock-out one or more native genes corresponding to pta, poxB, ldhA, adhE, ptsG, sdhA, and iclR.

In one embodiment, the biosynthetic pathway is for producing adipic acid via whole cell bioconversion from supplied medium to long chain free fatty acids (C₁₂-C₁₆) and comprises combinations of
a heterologously expressed or increased native activity of a 6-oxohexanoic acid dehydrogenase
a heterologously expressed or increased native activity of an omega exo fatty acid dehydrogenase
a heterologously expressed or increased native activity of a 6-hydroxyhexanoic acid dehydrogenase
a heterologously expressed or increased native activity of an omega hydroxyl fatty acid dehydrogenase
a heterologously expressed or increased native activity of a hexanoate synthase
a heterologously expressed or increased native activity of a monooxygenase
a heterologously expressed or increased native activity of a monooxygenase reductase
a heterologously expressed or increased native activity of a fatty acid oxidase
a heterologously expressed or increased native activity of an acyl-CoA ligase
a heterologously expressed or increased native activity of an acyl-CoA oxidase
a heterologously expressed or increased native activity of an enoyl-CoA reductase
a heterologously expressed or increased native activity of a 3-L-hydroxyacyl-CoA dehydrogenase
a heterologously expressed or increased native activity of an acetyl-CoA C-acetyltransferase

In one embodiment, the biosynthetic pathway is for producing muconic acid and comprises
a native or heterologously expressed pathway comprising a 2-dehydro-3-deoxy-D-arabinoheptonate 7-phosphate (DAHP) synthase, a 3-dehydroquinate synthase, and a 3-dehydroxyquinate dehydratase
a dehydroshikimic acid dehydratase (e.g. PobA from Pseudomonas putida KT2440)
a protocatechuate decarboxylase (e.g. AroY from Klebsiella pneumoniae)
a catechol 1,2-dioxygenase (e.g. CatA from Acinetobacter sp. ADP1)

The bacterial cell may further be modified to increase the flux toward precursors for DAHP (erythrose 4-phosphate and phosphoenolpyruvate), such as by knock-down or knock-out of genes corresponding to E. coli ptsH, ptsI, crr, and pykF; by overexpressing genes corresponding to ubiC, aroF, aroE, and aroL (or feedback-resistant mutants thereof), or combinations thereof.

Some bacteria contain a native pathway for production of a diacid, avoiding the necessity for a recombinant pathway. These include, for example, Actinobacillus succinogenes (succinic acid), Mannheimia succiniproducens (succinic acid), Thermobifida fusca (malic acid), and Escherichia coli (fumaric and malic acids), among numerous others.

3) Processes

In one aspect, there is provided a process for preparing a recombinant bacterial cell, e.g., an E. coli cell. Also provided is a process for improving the tolerance of a bacterial cell, e.g., an E. coli cell, to a diacid. Also provided is a method of identifying a bacterial cell which is tolerant to at least one diacid. Also provided is a process for preparing a recombinant bacterial cell, e.g., an E. coli cell, for producing a diacid.

These processes may comprise one or more steps of genetically modifying a bacterial cell to knock-down or knock-out one or more endogenous genes of any aspect or embodiment of the Group 1 modifications and/or introducing one or more mutations in the endogenous protein(s) or gene(s) of any Group 2 aspect or embodiment. This can be achieved by, e.g., transforming the bacterial cell with genetic constructs, e.g., vectors, antisense nucleic acids or siRNA, which result, e.g., in the knock-out or knock-down of a gene, introduce a mutation into an endogenous gene, or which encode the mutated protein from a transgene.

The genetic constructs, particularly vectors, can also comprise suitable regulatory sequences, typically nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters (e.g., constitutive promoters or inducible promoters), translation leader sequences, introns, polyadenylation recognition sequences, RNA processing sites, effector binding sites and stem-loop structures.

Alternatively, bacterial cells can be exposed to selection pressure (as described in the Examples) or to conditions which introduce random mutations in endogenous genes, and bacterial cells which comprise one or more Group 1 and/or Group 2 modifications according to any preceding aspects and embodiments can then be identified. Typically, this involves preparing a population of the genetically modified bacterial cell, having different Group 1 and/or Group 2 modifications, and then selecting from this population any bacterial cell which has an improved tolerance to a diacid at a predetermined concentration.

In one specific embodiment, the Group 1 modification is a knock-down or knock-out of one or more endogenous genes selected from kgtP, ybjL, proV, proW, proX, proQ, cspE, rfaE, yfbP, yfjM, pstS, pstA, pstB, pstC, rph, rpoS, sspA, tdk, uvrB, ycjG, and yeaR or, e.g., a knock-down or knock-out of kgtP in combination one or more other genes, e.g., ybjL, proV and/or sspA. In one specific embodiment, the Group 2 modification is a mutation in at least one endogenous protein or gene selected from SpoT, PolB, RpoC, RpoB, Rnt or SapC, such as e.g., at least one mutant protein selected from the group consisting of SpoT-V422A, SpoT-A451D, SpoT-A451V, SpoT-W457C, SpoT-N454H, SpoT-D580Y, SpoT-R236L, SpoT-R236S, SpoT-M247K, SpoT-NIR(601-603)S, SpoT-T442I, SpoT-S434L, PolB-R477G, RpoC-H419P, RpoC-P64L, RpoB-K203T, Rnt-Q179P, Rnt-A27T, Rnt-F194L, Rnt-A180T, SapC-G79W; and/or a mutation which increases the expression of PyrE, such as, e.g. a mutation in rph or the pyrE/rph intergenic region.

The processes may further comprise
a step of selecting any bacterial cell which has an improved tolerance to a diacid at a predetermined concentration in the medium, such as at least 1 g/L, such as at least 2 g/L, or higher, such as at least 5 g/L or higher, such as at least 10 g/L or higher, such as at least 20 g/L or higher, such as at least 40 g/L or higher, such as at least 75 g/L or higher, such as at least 100 g/L or higher;
an optional step of introducing a recombinant biosynthetic pathway for producing the diacid; or
both of the above steps, in any order.

In one embodiment, the diacid is glutaric acid, and the predetermined concentration is at least 2 g/L or higher, such as at least 5 g/L or higher, such as at least 10 g/L or higher, such as at least 20 g/L or higher, such as at least 40 g/L or higher, such as at least 75 g/L or higher, such as at least 100 g/L or higher. In one embodiment, the diacid is adipic acid, and the predetermined concentration is at least 2 g/L or higher, such as at least 5 g/L or higher, such as at least 10 g/L or higher, such as at least 20 g/L or higher, such as at least 40 g/L or higher, such as at least 75 g/L or higher, such as at least 100 g/L or higher. In one embodiment, the diacid is pimelic acid, and the predetermined concentration is at least 2 g/L or higher, such as at least 5 g/L or higher, such as at least 10 g/L or higher, such as at least 20 g/L or higher, such as at least 30 g/L or higher, such as at least 45 g/L or higher, such as at least 75 g/L or higher. In one embodiment, the diacid is sebacic acid, and the predetermined concentration is at least 2 g/L or higher, such as at least 5 g/L or higher, such as at least 10 g/L or higher, such as at least 20 g/L or higher, such as at least 30 g/L or higher, such as at least 45 g/L or higher, such as at least 75 g/L or higher.

In a particular embodiment, the predetermined concentration is at most 20 g/L, such as at most 30 g/L, such as at most 50 g/L, such as at most 75 g/L, such as at most 100 g/L, such as at most 150 g/L.

Assays for assessing the tolerance of a modified bacterial cell to a diacid typically evaluate the growth rate, lag time, or both, of the bacterial cell at predetermined concentrations for the diacid in question, typically as compared to a control. Preferably, the control is the native or unmodified parent cell or strain, and an improved tolerance is identified as an improved growth rate, a reduced lag-time or both. For example, an improved growth rate can be at least 5%, such as at least 10%, such as at least 20%, such as at least 50%, such as at least 75% higher than that of the control, while a reduced lag time can be at least 10%, such as at least 20%, such as at least 50%, such as at least 75%, such as at least 90% shorter than that of the control. Specific assays are described, in detail, in the Examples.

Also provided is a method of producing a diacd, comprising culturing the bacterial cell obtained by any one of these methods, or the bacterial cell of any preceding aspect or embodiment, under conditions where the diacid is produced. Typically, these conditions include the presence of a suitable carbon source or mixes of different suitable carbon sources. Non-limiting examples of suitable carbon sources include, e.g., sucrose, D-glucose, D-xylose, L-arabinose, glycerol; raw carbon feedstocks such as crude glycerol and cane syrup; as well as hydrolysates produced from cellulosic or lignocellulosic materials. For further details see, e.g., Adkins et al., 2013; Park et al., 2013; Yu et al., 2014; Cheong et al., 2016; Deng and Mao, 2015; WO 2011/003034 A2 (Verdezyne); Curran et al., 2013; Sengupta et al., 2015; and Zhang et al., 2015.

4) Compositions

A bacterial cell which has an increased tolerance to a diacid can be useful as a production host for the diacid. Bacterial cells according to the invention may have an increased growth rate, a decreased lag time, or both, in the diacid. For example, the bacterial cell may have Group 1 and/or Group 2 modifications providing for an increased growth rate, a reduced lag time, or both, of the cell in at least one diacid, e.g., in glutaric acid, adipic acid, succinic acid, muconic acid, fumaric acid, itaconic acid, malic acid, malonic acid, maleic acid, glucaric acid, pimelic acid, suberic acid, sebacic acid, 2,5-furandicarboxylic acid, terephthalic acid, or azelaic acid, mesaconic acid, citraconic acid, tartaric acid, tartronic acid, diaminopimelic acid and/or glutaconic acid.

In one aspect, there is provided a composition comprising a plurality of bacterial cells according to any aspect or embodiment described herein, e.g., an in vitro culture of such bacterial cells, optionally in a suitable culture medium and/or a chemically-defined medium comprising a carbon source. In one embodiment, the composition is substantially homogenous with respect to the bacterial cells.

In one aspect, there is provided a composition comprising a plurality of bacterial cells according to any preceding aspect or embodiment and a diacid. In one embodiment, the diacid is present at a concentration at which the genetic modification(s) and/or mutant(s) comprised in the bacterial cells results in an improved tolerance as compared to the parent bacterial cells, e.g., wild-type or native bacterial cells. The concentrations at which bacterial cells according to the invention have improved tolerance are shown in Example 1, e.g., in “Cross-compound tolerance testing”. Typically, the concentration of the diacid is at least 1 g/L, such as at least 2 g/L, or higher, such as at least 5 g/L or higher, such as at least 10 g/L or higher, such as at least 20 g/L or higher, such as at least 40 g/L or higher, such as at least 45 g/L or higher, such as at least 75 g/L or higher, such as at least 100 g/L or higher.

In one aspect, there is provided a composition comprising
a diacid at a concentration of at least 1 g/L, such as at least 2 g/L, or higher, such as at least 5 g/L or higher, such as at least 10 g/L or higher, such as at least 20 g/L or higher, such as at least 40 g/L or higher, such as at least 45 g/L or higher, such as at least 75 g/L or higher, such as at least 100 g/L or higher; and
a plurality of bacterial cells according to any preceding aspect or embodiment.

In separate and specific embodiments, the diacid is glutaric acid, adipic acid, pimelic acid and sebacic acid, respectively. In other specific embodiments, the diacid present in the composition is at least 45 g/L fumaric acid, at least 45 g/L itaconic acid, at least 55 g/L malic acid, at least 50 g/L succinic acid, at least 45 g/L pimelic acid, and at least 38 g/L sebacic acid, respectively.

As described in Example 1; “Cross-compound tolerance testing,” genetic modifications according to the invention also conferred tolerance to other chemicals, such as to other carboxylic acids (glutarate and adipate; hexanoate, octanoate, isobutyrate, glutarate and p-coumarate), to diamines (e.g., HMDA, putrescine) and diols (2,3-butanediol, 1,2-propanediol). Accordingly, in one embodiment, there is provided a composition comprising a plurality of bacterial cells according to any preceding aspect or embodiment, and a chemical selected from the following, at at least the indicated concentration:

| | | |
|---|---|---|
| butanol | 1.4% | v/v |
| glutarate | 40 | g/L |
| p-coumarate | 7.5 | g/L |
| putrescine | 32 | g/L |
| HMDA | 32 | g/L |
| adipate | 45 | g/L |
| isobutyrate | 7.5 | g/L |
| hexanoate | 3 | g/L |
| octanoate | 8 | g/L |
| 2,3-butanediol | 6% | v/v |
| 1,2-propanediol | 6% | v/v |

| | |
|---|---|
| sodium chloride | 0.6M |

Preferably, the bacterial cells are of the Escherichia, Lactobacillus, Lactococcus, Bacillus, Pseudomonas, Corynebacterium, Deinococcus or Ralstonia genera, such as, e.g., E. coli cells, and comprise
a) at least one genetic modification which reduces expression of an endogenous gene selected From the group consisting of kgtP, ybjL, proV, proW, proX, proQ, cspE, rfaE, yfbP, yfjM, pstS, pstA, pstB, pstC, rph, rpoS, sspA, tdk, uvrB, ycjG, and yeaR, or a combination of any thereof;
b) a mutation in one or more of SpoT, PolB, RpoC, RpoB, Rnt and SapC; an increased expression of one or more of SpoT, PolB, RpoC, RpoB, Rnt and SapC; and/or a mutation in rph or the pyrE/rph intergenic region which increases the expression of pyrE, or
c) a combination of (a) and (b).

Assays for assessing the tolerance of a modified bacterial cell to a selected diacid typically evaluate the growth rate, lag time, or both, of the bacterial cell at one or more predetermined concentrations of the compound, typically as compared to a control (e.g., no compound). The predetermined concentrations(s) could be, for example, 1, 2, 5, 10, 20, 40, 45, 75, or 100 g/L. Preferably, the control is the native or unmodified parent cell or strain, and an improved tolerance is identified as an improved growth rate, a reduced lag-time or both. For example, an improved growth rate can be at least 5%, such as at least 10%, such as at least 20%, such as at least 50%, such as at least 75%, such as at least 100%, such as at least 200%, such as at least 300%, such as at least 500%, such as at least 1000%, such as at least 10000% higher than that of the control; while a reduced lag time can be at least 10%, such as at least 20%, such as at least 50%, such as at least 75%, such as at least 90% shorter than that of the control. Indeed, in some cases the native or unmodified parent cell cannot grow at all in a concentration of the diacid that the modified bacterial cell can grow in. Specific assays are described, in detail, in the Examples.

5) Bacterial Cells

Also provided are strains, clones and other progeny of the bacterial cells of these and other aspects and embodiments, as well as cell cultures of such bacterial cells or strains. Typically, as used herein, a “strain” typically refers to a group of cells which are descendants of a initial single colony of parent cells whereas a “clone” is a group of cells which are the descendants of an initial genetically modified single parent cell.

Non-limiting examples of bacterial cells suitable for modification according to any one of the aspects and embodiments described herein include bacteria of the Escherichia, Lactobaccillus, Lactococcus, Corynebacterium, Bacillus, Ralstonia, Clostridia, Deinococcus or Pseudomonas genera, such as from the Escherichia, Lactobacillus, Lactococcus, Bacillus, Pseudomonas, Corynebacterium, Deinococcus or Ralstonia genera. In one embodiment, the bacterial cell is an E. coli cell, such as a cell of the commercially available and/or fully characterized strains K-12 MG1655, BW25113, BL21, BL21(DE3), K-12 W3110, W, JM109, or Crooks (ATCC 8739). In a specific embodiment, the bacterial cell is derived from an E. coli K12 strain. In another embodiment, the bacterial cell is a Lactobacillus cell, such as a cell of the commercially available and/or fully characterized strains Lactobacillus plantarum JDM1, Lactobacillus plantarum WCFS1, and Lactobacillus plantarum NCIMB 8826. In another embodiment, the bacterial cell is a Lactococcus cell, such as a cell of the commercially available and/or fully characterized strains Lactococcus lactis lactis CV56, Lactococcus lactis lactis NIZO B40, and Lactococcus lactis cremoris NZ9000. In another embodiment, the bacterial cell is a Bacillus cell, such as a cell of the commercially available and/or fully characterized strains Bacillus subtilis 168 and Bacillus subtilis PY79. In one embodiment, the bacterial cell is a Pseudomonas cell, such as a cell of the commercially available and/or fully characterized strain Pseudomonas putida KT2440. In another embodiment, the bacterial cell is a Ralstonia cell, such as a cell of the commercially available and/or fully characterized strains Ralstonia eutropha H16 and Ralstonia eutropha JMP134. In another embodiment, the bacterial cell is a Corynebacterium cell, such as a cell of the commercially available and/or fully characterized strains 534 (ATCC 13032), K051, MB001, R, SCgG1, and SCgG2. In another embodiment, the bacterial cell is a Deinococcus cell, such as a D. radiodurans or D. geothermalis cell, such as a cell of the commercially available and/or fully characterized strain D. radiodurans R1.

While aspect and embodiments relating to bacterial cells herein typically refer to genes or proteins according to their designation in E. coli, for bacterial cells of another family or species, it is within the level of skill in the art to identify the corresponding gene or protein, i.e., the ortholog and/or paralog, in the other family or species, typically by identifying sequences having moderate or high homology to the E. coli sequence, optionally taking the function of the protein expressed by the gene and/or the locus of the gene in the genome into account, Table 2 sets out the function of the protein encoded by each specific gene, the corresponding E.C. number (if applicable), its locus in the E. coli K-12 MG1655 genome and the SEQ ID number of the coding or non-coding sequence and, where applicable, the encoded amino acid sequence.

Tables 3 and 4 set out some examples of homologs or orthologs in selected organisms, identified in a preliminary and non-limiting analysis. Indeed, homologs or orthologs of these proteins exist also in other bacteria, and other homologs or orthologs not identified in this preliminary search can exist in the species listed in Table 3. The skilled person is well-familiar with different searching and/or screening methods for identifying homologs or orthologs across different species. To briefly summarize some of the preliminary findings in Table 3:
KgtP, ProV, ProW, CspE, RfaE, PstS, pstA, pstB, pstC, UvrB, SpoT, RpoB, RpoC, SapC, and PyrE are widely conserved and were identified in all organisms. ProW and ProX appear to be fused in Lactococcus lactis and Lactobacillus plantarum.
Two YbjL homologs or orthologs were identified in Corynebacterium glutamicum.
Rph was found to be conserved in all organisms with the exception of Lactococcus lactis.
ProQ was only found in Pseudomonas putida, indicating it is likely only conserved in the Gammaproteobacteria.
RpoS is likely only conserved as an alternative sigma factor to RpoD in Gram-negative bacteria.
SspA was found to be conserved in Gram-negative organisms.
Tdk was found to be conserved between E. coli and only certain Gram-positive organisms in Table 3 (B. subtilis, L. lactis, L. plantarum)
YcjG has a conserved annotated function in Bacillus subtilis and has homology to annotated muconate/chloromuconate cycloisomerases, which may be another activity of the enzyme, in the Gram-negative organisms (P. putida, R. eutropha) and C. glutamicum.
PolB is conserved in the Gram-negative organisms (P. putida, R. eutropha)
Rnt has a high degree of identity and with similar annotated functions to a protein in P. putida, and is a homolog of annotated DNA polymerases in B. subtilis, L. plantarum, L. lactis, and R. eutropha.

TABLE 2
Protein function and Locus IDs

| E. coli gene | | E.C. | Locus | |
|---|---|---|---|---|
| designation | Protein function | number | ID | SEQ ID NO: |
| kgtP | α-ketoglutarate:H⁺ symporter | N/A | b2587 | 1 |
| ybjL | inner membrane protein YbjL | N/A | b0847 | 2 |
| proV | glycine betaine/proline ABC | 3.6.3.32 | b2677 | 3 |
| | transporter - ATP binding | | | |
| | subunit | | | |
| proW | glycine betaine/proline ABC | 3.6.3.32 | b2678 | 4 |
| | transporter - membrane | | | |
| | subunit | | | |
| proX | glycine betaine/proline ABC | 3.6.3.32 | b2679 | 5 |
| | transporter - periplasmic | | | |
| | binding protein | | | |
| proQ | RNA chaperone, involved in | N/A | b1831 | 6 |
| | posttranscriptional control of | | | |
| | ProP levels | | | |
| cspE | transcription antiterminator and | N/A | b0623 | 7 |
| | regulator of RNA stability | | | |
| rfaE | fused heptose-7-phosphate | 2.7.7.70, | b3052 | 8 |
| | kinase/heptose-1-phosphate | 2.7.1.167 | | |
| | adenyltransferase | | | |
| yfbP | predicted protein | N/A | b2275 | 9 |
| yfjM | CP4-57 prophage; predicted | N/A | b2629 | 10 |
| | protein | | | |
| pstS | phosphate ABC transporter - | 3.6.3.27 | b3728 | 11 |
| | periplasmic binding protein | | | |
| pstA | phosphate ABC transporter - | 3.6.3.27 | b3726 | 34 |
| | membrane subunit | | | |
| pstB | phosphate ABC transporter - | 3.6.3.27 | b3725 | 35 |
| | ATP binding subunit | | | |
| pstC | phosphate ABC transporter - | 3.6.3.27 | b3727 | 36 |
| | membrane subunit | | | |
| rph | RNAse PH | 2.7.7.56 | b3643 | 12 |
| rpoS | RNA polymerase, sigma S | N/A | b2741 | 13 |
| | (sigma 38) factor | | | |
| sspA | stringent starvation protein A | N/A | b3229 | 14 |
| tdk | thymidine kinase/deoxyuridine | 2.7.1.145, | b1238 | 15 |
| | kinase | 2.7.1.21 | | |
| uvrB | DNA repair; excision nuclease | 3.1.25.— | b0779 | 16 |
| | subunit B | | | |
| ycjG | L-Ala-D/L-Glu epimerase | 5.1.1.20 | b1325 | 17 |
| yeaR | conserved protein | N/A | b1797 | 18 |
| spoT | guanosine 3′-diphosphate 5′- | 3.1.7.2 | b3650 | 19 (DNA) |
| | triphosphate 3′-diphosphatase | | | 20 (protein) |
| | [multifunctional] | | | |
| polB | DNA polymerase II | 3.1.11.—, | b0060 | 21 (DNA) |
| | | 2.7.7.7 | | 22 (protein) |
| rpoB | RNA polymerase, β subunit | 2.7.7.6 | b3987 | 23 (DNA) |
| | | | | 24 (protein) |
| rpoC | RNA polymerase, β′ subunit | 2.7.7.6 | b3988 | 25 (DNA) |
| | | | | 26 (protein) |
| rnt | RNase T | 3.1.13.— | b1652 | 27 (DNA) |
| | | | | 28 (protein) |
| sapC | integral membrane protein | N/A | b1292 | 29 (DNA) |
| | SapC of predicted ABC | | | 30 (protein) |
| | transporter | | | |
| pyrE | Orotate | 2.4.2.10 | b3642 | 31(DNA) |
| | phosphoribosyltransferase | | | 32 (protein) |
| pyrE/rph | — | — | — | 33 |
| intergenic | | | | |
| region | | | | |

TABLE 3
Homologs or orthologs identified by protein BLAST (BLASTP) of E. coli K-12 MG1655 proteins against protein databases from selected
reference organisms. Hits with the largest e-value are shown, and hits are only shown when the e-value < 1.0. Hit proteins with
e-value < 0.1 (non-italicized) are deemed more probable of having the same or similar function as the E. coli protein.

| Protein | | | | | Ralstonia | Corynebacterium |
|---|---|---|---|---|---|---|
| (# of | B. subtilis | P. putida | L. plantarum | L. lactis | eutropha | glutamicum |
| residues) | 168 | KT2440 | JDM1 | KF147 | H16 | ATCC 13032 |
| KgtP | 24-27% identity | 56-70% identity | 23-28% identity | 23% identity | 33-37% identity | 28-33% identity |
| (432 aa) | (339-420 aa) | (415-416 aa) | (176-355 aa) | (347 aa) | (337-381 aa) | (392-443 aa) |
| | “metabolite | “major facilitator | “arabinose | “arabinose- | “major facilitator | “integral |
| | transporter” | superfamily | transport protein” | proton symporter” | superfamily | membrane |
| | (NP_388707.1), | metabolite/H+ | (YP_003064425.1), | (NP_003354045.1) | transporter MHS | transport |
| | “metabolite | symporter” | “transport | | family protein” | protein” |
| | transport | (NP_743537,1, | protein” | | (YP_725964.1, | (NP_602106.1). |
| | protein YwtG” | NP_743559.1) | (YP_003063609.1), | | YP_725210.1), | “proline- |
| | (NP_391464.2), | | “sugar transport | | “MFS family | betaine |
| | “metabolite | | protein” | | transporter” | transporter” |
| | transport | | (YP_003064460.1) | | (YP_725057.1) | (NP_602258.1), |
| | protein YncC” | | | | | “major |
| | (NP_389645.2), | | | | | facilitator |
| | “major myo- | | | | | superfamily |
| | inositol | | | | | permease” |
| | transporter | | | | | (NP_599668.1, |
| | IolT” | | | | | NP_599535.2, |
| | (NP_388504.1) | | | | | NP_600684.1) |
| YbjL | 28% identity | 31% identity | 34% identity | 25% identity | 31% identity | 25% identity |
| (561 aa) | (116 αα) | (123 aa) | (68 αα) | (76 αα) | (566 aa) | (548 aa) |
| | “sulfate | “potassium/ | “sugar | “chloride | “aspartate: | “permease” |
| | transporter | proton | transport protein” | channel protein” | alanine | (NP_601414.1); |
| | YvdB” | antiporter” | (YP_003064460.1) | (YP_003354224.1) | antiporter” | 25% identity |
| | (NP_391346.1) | (NP_747167.2) | | | (YP_726110.1); | (550 aa) |
| | | | | | 31% identity | “hypothetical |
| | | | | | (550 aa) | protein |
| | | | | | “permease” | NCgl0565” |
| | | | | | (YP_725451.1) | (NP_599826.1) |
| ProV | 51% identity | 36-51% identity | 34-44% identity | 39-48% identity | 33-44% identity | 35-40% identity |
| (400 aa) | (390 aa) | (222-352 aa) | (224-361 aa) | (247-392 aa) | (195-354 aa) | (226-283 aa) |
| | “glycine/ | “glycine | “glycine/betaine/ | “glycine | “ABC transporter | “ABC transporter |
| | betaine ABC | betaine/L-proline | carnitine/choline | betaine/carnitine/ | ATPase” | ATPase” |
| | transporter | ABC transporter | ABC transporter | choline ABC | (YP_724876.1, | (NP_599870.1, |
| | ATP-binding | ATP-binding | ATP-binding | transporter ATP- | YP_726702.1, | NP_601662.1, |
| | protein” | subunit” | protein” | binding protein” | YP_725457.1, | NP_599673.1, |
| | (NP_388180.2) | (NP_742461.1), | (YP_003062931.1, | (YP_003353988.1), | YP_725326.1, | NP_599959.1, |
| | | “glycine | YP_003061916.1) | “glycine/betaine | YP_726845.1, | NP_600605.1, |
| | | betaine/L-proline | | ABC transporter | YP_724565.1, | NP_600550.1), |
| | | ABC transporter | | ATP-binding | YP_727745.1, | “glutamate ABC |
| | | ATPase/permease | | protein” | YP_727463.1, | transporter |
| | | fusion protein” | | (YP_003353316.1) | YP_725974.1, | ATPase” |
| | | (NP_744918.1), | | | YP_724993.1, | (NP_601157.1), |
| | | “glycine | | | YP_727203.1, | “ABC |
| | | betaine/L-proline | | | YP_725812.1, | transporter |
| | | ABC transporter | | | YP_726707.1), | duplicated |
| | | ATPase” | | | “ABC-type | ATPase” |
| | | (NP_743029.1) | | | transporter, | (NP_601199.1) |
| | | | | | ATPase | |
| | | | | | component” | |
| | | | | | (YP_727764.1) | |
| ProW | 48% identity | 40-53% identity | 34-40% identity | 35-47% identity | 27-34% identity | 25-30% identity |
| (354 aa) | (275 aa) | (206-265 aa) | (161-169 aa) | (155-284 aa) | (152-194 aa) | (157-232 aa) |
| | “glycine | “glycine | “glycine | “glycine | “ABC | “ABC |
| | betaine | betalne/L-proline | betaine/carnitine/ | betaine/carnitine/ | transporter | transporter |
| | transport | ABC transporter | choline ABC | choline ABC | permease” | permease” |
| | system | permease” | transporter, | transporter | (YP_725456.1, | (NP_600676.1, |
| | permease | (NP_742462.1), | substrate binding | permease/substrate- | YP_725454.1, | NP_600445.1) |
| | protein OpuAB” | “binding protein- | and permease | binding protein” | YP_726844.1, | |
| | (NP_388181.1) | dependent | protein” | (YP_003353987.1), | YP_724987.1), | |
| | | transport system | (YP_003061915.1), | “glycine betaine | “ABC-type | |
| | | inner membrane | “glycine | ABC transporter | transporter, fused | |
| | | protein” | betaine/carnitine/ | permease/substrate- | periplasmic and | |
| | | (NP_745696.1), | choline ABC | binding protein | permease | |
| | | “glycine | transporter, | (YP_003353317.1) | components” | |
| | | betaine/L-proline | permease protein” | | (YP_726088.1) | |
| | | ABC transporter | (YP_003062932.1) | | | |
| | | ATPase/permease | | | | |
| | | fusion protein” | | | | |
| | | (NP_744918.1) | | | | |
| ProX | 25% identity | 21-27% identity | Not found | 22% identity | 29% identity | Not found |
| (330 aa) | (124 aa) | (155-327 aa) | | (279 aa) | (75 αα) | |
| | “glycine | “glycine betaine | | “glycine | “RND | |
| | betaine-binding | ABC transporter | | betaine/carnitine/ | superfamily | |
| | protein OpuAC” | substrate- | | choline ABC | exporter” | |
| | (NP_388182.1) | binding protein” | | transporter | (YP_725351.1) | |
| | | (NP_745695.1, | | permease/ | | |
| | | NP_744919.1), | | substrate-binding | | |
| | | “glycine/betaine- | | protein” | | |
| | | binding protein” | | (YP_003353987.1) | | |
| | | (NP_742246.1) | | | | |
| ProQ | 24% identity | 27% identity | Not found | Not found | 29% identity | 33% identity |
| (232 aa) | (128 αα) | (115 aa) | | | (76 αα) | (66 αα) |
| | “hypothetical | “ProQ | | | “dehydrogenase” | “elongation |
| | protein | activator of | | | (YP_724959.1) | factor Ts” |
| | BSU32070” | osmoprotectant | | | | (NP_601230.1) |
| | (NP_391087.1) | transporter | | | | |
| | | ProP” | | | | |
| | | (NP_744331.1) | | | | |
| CspE | 61-68% identity | 54-62% identity | 65-69% identity | 62-66% identity | 62% identity | 63-65% identity |
| (69 aa) | (62-64 aa) | (61-68 aa) “cold | (61-62 aa) | (61-62 aa) | (61 aa) | (65 aa) |
| | “cold shock | shock protein | “cold shock | “cold-shock | “cold shock | “cold shock |
| | protein | CspA” | protein | protein” | protein, DNA- | protein” |
| | CspB” | (NP_743679.1), | CspP” | (YP_003352748.1, | binding” | (NP_599426.1, |
| | (NP_388791.1), | “cold-shock | (YP_003062538.1), | YP_003353646.1, | (YP_727497.1) | NP_599560.1) |
| | “cold shock | domain-contain | “cold shock | YP_003352635.1, | | |
| | protein CspC” | protein” | protein CspL” | YP_003354678.1) | | |
| | (NP_388393.1), | (NP_743146.1, | (YP_003061635.1, | | | |
| | “cold shock | NP_743260.1), | YP_003061614.1), | | | |
| | protein CspD” | “cold shock | “cold shock | | | |
| | (NP_390076.1) | protein CspA” | protein CspC” | | | |
| | | (NP_744611.1), | (YP_003062410.1) | | | |
| | | “cold-shock | | | | |
| | | domain-contain | | | | |
| | | protein, partial” | | | | |
| | | (NP_743369.1), | | | | |
| | | “cold-shock | | | | |
| | | protein CspD” | | | | |
| | | (NP_746140.1) | | | | |
| RfaE | 32% identity | 57% identity | 34% identity | 35% identity | 49% identity | 28% identity |
| (477 aa) | (133 aa) | (474 aa) | (128 aa) | (98 aa) | (312 aa) | (120 aa) |
| | “glycerol-3- | “bifunctional | “glycerol-3- | “glycerol-3- | “D-beta-D- | “ribokinase |
| | phosphate | heptose-7- | phosphate | phosphate | heptose 7- | sugar kinase” |
| | cytidylyltrans- | phosphate | cytidylyltransferase” | cytidylyltransferase” | phophosphate | (NP_599410.1) |
| | ferase”, 24-26% | kinase/heptose- | (YP_003062640.1), | (YP_003352686.1); | kinase” | |
| | identity | 1-phosphate | 24% identity | 33% identity | (YP_725318.1) | |
| | (228-327 aa) | adenyltransferase” | (240-309 aa) | (147 aa) | | |
| | “ribokinase” | (NP_747037.1) | “ribokinase” | “glycerol-3- | | |
| | (NP_391473.1), | | (YP_003064511.1, | phosphate | | |
| | “sugar kinase | | YP_003063470.1) | cytidyltransferase” | | |
| | YdjE” | | | (YP_003353407.1) | | |
| | (NP_388498.1), | | | | | |
| | “fructosamine | | | | | |
| | kinase FrlD” | | | | | |
| | (NP_391137.1) | | | | | |
| YfbP | 28% identity | 24% identity | Not found | 24% identity | 29% identity | 33% identity |
| (282 aa) | (68 αα) | (148 αα) | | (106 αα) | (106 αα) | (39 aa) |
| | “sensor | “tryptophan | | “multimodular | “O-linked N- | “hypothetical |
| | histidine | synthase subunit | | transpeptidase- | acetylglucosamine | protein |
| | kinase” | alpha” | | transglycosylase | transferase OGT” | NCgl0374” |
| | (NP_391844.1) | (NP_742252.1) | | Pbp2A” | (YP_724894.1) | (NP_599633.1) |
| | | | | (YP_003354748.1) | | |
| YfjM | Not found | 30% identity | 26% identity | Not found | Not found | Not found |
| (87 aa) | | (54 αα) | (53 αα) | | | |
| | | “ABC transporter | “small heat | | | |
| | | substrate- | shock protein” | | | |
| | | binding protein” | (YP_003064271.1) | | | |
| | | (NP_744413.1) | | | | |
| PstS | 26% identity | 39% identity | 27-28% identity | 26% identity | 64% identity | 30% identity |
| (346 aa) | (229 aa) | (339 aa) | (224-318 aa) | (232-251 aa) | (347 aa) | (282 aa) |
| | “phosphate- | “phosphate | “phosphate | “phosphate | “ABC | “ABC |
| | binding protein | ABC transporter | ABC transporter | ABC transporter | transporter | transporter |
| | PstS” | substrate- | substrate-binding | substrate-binding | periplasmic | periplasmic |
| | (NP_390378.1) | binding protein” | protein” | protein” | protein” | component” |
| | | (NP_744800.1) | (YP_003062200.1, | (YP_003354292.1, | (YP_726901.1) | (NP_601773.1) |
| | | | YP_003062190.1) | YP_003354291.1) | | |
| Rph | 58% identity | 69% identity | Not found | 24-27% identity | 62% identity | 59% identity |
| (228 aa) | (222 aa) | (228 aa) | | (59-207 aa in | (221 aa) | (217 aa) |
| | “ribonuclease | “ribonuclease | | stretches) | “ribonuclease | “ribonuclease |
| | PH” | PH” | | “polyribo- | PH” | PH” |
| | (NP_390715.1) | (NP_747395.1) | | nucleotide | (YP_725462.1) | (NP_601703.2) |
| | | | | nucleotidyltrans- | | |
| | | | | ferase” | | |
| | | | | (YP_003354448.1) | | |
| RpoS | 43% identity | 76% identity | 44% identity | 40% identity | 52% identity | 39% identity |
| (330 aa) | (321 aa) “RNA | (277 aa) “RNA | (271 aa) “RNA | (288 aa) “RNA | (280 aa) “RNA | (316 aa) “RNA |
| | polymerase | polymerase | polymerase | polymerase | polymerase | polymerase |
| | sigma factor | sigma factor | sigma factor | sigma factor | sigma factor | sigma factor |
| | RpoD” | RpoS” | RpoD” | RpoD” | RpoS” | SigB” |
| | (NP_390399.2) | (NP_743780.1) | (YP_003063237.1) | (YP_003352999.1) | (YP_726836.1) | (NP_601125.1), |
| | | | | | | 36% identity |
| | | | | | | (304 aa) “RNA |
| | | | | | | polymerase |
| | | | | | | sigma factor” |
| | | | | | | (NP_601117.2) |
| SspA | Not found | 57% identity | 45% identity | Not found | 46% identity | 56% identity |
| (212 aa) | | (200 aa) | (22 αα) | | (203 aa) | (16 αα) |
| | | “stringent | “hypothetical | | “stringent | “hypothetical |
| | | starvation | protein | | starvation protein | protein |
| | | protein A” | JDM1_{—}0823” | | A” (YP_727831.1) | NCgl2333” |
| | | (NP_743480.1) | (YP_003062407.1) | | | (NP_601617.1) |
| Tdk | 30% identity | Not found | 45% identity | 49% identity | 25% identity | Not found |
| (205 aa) | (184 aa) | | (152 aa) | (187 aa) | (132 αα) | |
| | “thymidine | | “thymidine | “thymidine | “prolyl-tRNA | |
| | kinase” | | kinase” | kinase” | synthetase” | |
| | (NP_391587.1) | | (YP_003063573.1) | (YP_003353039.1) | (YP_727689.1) | |
| UvrB | 59% identity | 69% identity | 59% identity | 55% identity | 66% identity | 56% identity |
| (673 aa) | (666 aa) | (670 aa) | (663 aa) | (693 aa) | (673 aa) | (668 aa) |
| | “UvrABC | “excinuclease | “exonuclease | “excinuclease | “excinuclease | “excinuclease |
| | system protein | ABC subunit B” | ABC subunit B” | ABC subunit B” | ABC subunit B” | ABC subunit B” |
| | B” | (NP_744125.1) | (YP_003062224.1) | (YP_003353008.1) | (YP_725661.1) | (NP_600587.1) |
| | (NP_391397.1) | | | | | |
| YcjG | 31% identity | 26% identity | 31% identity | 24% identity | 26% identity | 27% identity |
| (321 aa) | (322 aa) | (337 aa) | (91 αα) | (338 aa) “O- | (324 aa) | (290 aa) |
| | “L-Ala- | “muconate and | “phosphopyruvate | succinylbenzoate | “muconate | “chloromuconate |
| | D/L-Glu | chloromuconate | hydratase” | synthase” | cyclo-isomerase” | cycloisomerase” |
| | epimerase” | cycloisomerase” | (YP_003063198.1) | (YP_003353203.1) | (YP_726435.1) | (NP_601602.2) |
| | (NP_389181.1) | (NP_745848.1) | | | | |
| YeaR | 26% identity | 24% identity | Not found | Not found | Not found | Not found |
| (119 aa) | (91 αα) | (41 αα) | | | | |
| | “hypothetical | “30S ribosomal | | | | |
| | protein | protein S12” | | | | |
| | BSU13060” | (NP_742615.1) | | | | |
| | (NP_389189.1) | | | | | |
| SpoT | 40% identity | 37-55% identity | 38% identity | 40% identity | 36-47% identity | 38% identity |
| (702 aa) | (719 aa) “GTP | (681-701 aa) | (741 aa) “GTP | (725 aa) “GTP | (674-720 aa) “GTP | (723 aa) |
| | pyrophosphokinase” | “(p)ppGpp | pyrophosphokinase” | pyrophosphokinase/ | pyrophosphokinase” | “guanosine |
| | (NP_390638.2) | synthetase I | (YP_003063260.1) | guanosine-3,5- | (YP_725468.1, | polyphosphate |
| | | SpoT/RelA” | | bis(diphosphate) 3- | YP_725845.1) | pyrophospho- |
| | | (NP_747403.1, | | pyrophospho- | | hydrolase/ |
| | | NP_743813.1) | | hydrolase” | | synthetase” |
| | | | | (YP_003352549.1) | | (NP_600866.1) |
| PolB | Not found | 68% identity | Not found | 32% identity | 68% identity | 35% identity |
| (783 aa) | | (784 aa) “DNA | | (63 αα) | (784 aa) “DNA | (55 αα) |
| | | polymerase II” | | “O-succinylbenzoate | polymerase II” | “rhodanese- |
| | | (NP_744541.1) | | synthase” | (YP_726151.1) | related |
| | | | | (YP_003353203.1) | | sulfurtransferase” |
| | | | | | | (NP_599306.1) |
| RpoB | 59% identity | 72% identity | 47-52% identity | 46-47% identity | 66% identity | 41-56% identity |
| (1342 aa) | (533 aa) “DNA- | (1360 aa) “DNA- | (304-953 aa in | (307-951 aa in | (1370 aa) | (238-616 aa in |
| | directed RNA | directed RNA | stretches) “DNA- | stretches) “DNA- | “DNA-directed | stretches) |
| | polymerase | polymerase | directed RNA | directed RNA | RNA polymerase | “DNA-directed |
| | subunit beta” | subunit beta” | polymerase | polymerase | subunit beta” | RNA |
| | (NP_387988.2) | (NP_742613.1) | subunit beta” | subunit beta” | (YP_727933.1) | polymerase |
| | | | (YP_003062426.1) | (YP_003354373.1) | | subunit beta” |
| | | | | | | (NP_599733.1) |
| RpoC | 50% identity | 75% identity | 44-51% identity | 48-51% identity | 67% identity | 46-50% identity |
| (1407 aa) | (1134 aa) | (1399 aa) “DNA- | (235-1061 aa in | (238-1043 aa in | (1397 aa) | (206-819 aa in |
| | “DNA-directed | directed RNA | stretches) “DNA- | stretches) “DNA- | “DNA-directed | stretches) |
| | RNA | polymerase | directed RNA | directed RNA | RNA polymerase | “DNA-directed |
| | polymerase | subunit beta'” | polymerase | polymerase | subunit beta'” | RNA |
| | subunit beta'” | (NP_742614.1) | subunit beta'” | subunit beta'” | (YP_727932.1) | polymerase |
| | (NP_387989.2) | | (YP_003062427.1) | (YP_003354372.1) | | subunit beta'” |
| | | | | | | (NP_599734.1) |
| Rnt | 27% identity | 63% identity | 28% identity | 29% identity | 27% identity | Not found |
| (215 aa) | (191 aa) “DNA | (198 aa) | (180 aa) “DNA | (206 aa) “DNA | (180 aa) “DNA | |
| | polymerase III | “Ribonuclease T” | polymerase III | polymerase III | polymerase III | |
| | PolC-type” | (NP_743246.1) | PolC” | subunit alpha” | subunit epsilon” | |
| | (NP_389540.1) | | (YP_003063293.1) | (YP_003354765.1) | (YP_726924.1) | |
| SapC | 28-30% identity | 42% identity | 25% identity | 23-27% identity | 34-37% identity | 31-38% identity |
| (296 aa) | (286-288 aa) | (281 aa) | (302 aa) | (219-306 aa) | (216-238 aa) | (215-230 aa) |
| | “oligopeptide | “binding- | “peptide ABC | “peptide ABC | “ABC | “ABC |
| | transport system | protein- | transporter | transporter | transporter | transporter |
| | permease | dependent | permease” | permease” | permease” | permease” |
| | protein AppC” | transport system | (YP_003062653.1) | (YP_003354430.1, | (YP_725975.1, | (NP_601522.1, |
| | (NP_389022.1), | inner membrane | | YP_003352873.1) | YP_726565.1, | NP_601635.1, |
| | “dipeptide | protein” | | | YP_726551.1, | NP_601198.1) |
| | transport system | (NP_743041.1) | | | YP_727395.1) | |
| | permease | | | | | |
| | protein DppC” | | | | | |
| | (NP_389177.1), | | | | | |
| | “oligopeptide | | | | | |
| | transport system | | | | | |
| | permease | | | | | |
| | protein OppC” | | | | | |
| | (NP_389027.1) | | | | | |
| PyrE | 25-34% identity | 67% identity | 29% identity | 30% identity | 56% identity | 29% identity |
| (213 aa) | (in stretches) | (213 aa) | (138 aa) | (131 aa) | (215 aa) | (139 aa) |
| | “orotate | “orotate | “orotate | “orotate | “orotate | “orotate |
| | phosphoribosyl- | phosphoribosyl- | phosphoribosyl- | phosphoribosyl- | phosphoribosyl- | phosphoribosyl- |
| | transferase” | transferase” | transferase” | transferase” | transferase” | transferase” |
| | (NP_389439.1) | (NP_747392.1) | (YP_003063746.1) | (YP_003353548.1) | (YP_724744.1) | (NP_601967.1) |

TABLE 4
Homologs or orthologs identified by protein BLAST (BLASTP) of E. coli K-12 MG1655 proteins against
protein databases from the two chromosomes and two endogenous plasmids of Deinococcus radiodurans
R1. Hits with the largest e-values are shown, and hits are only shown when the e-value < 1.0.

| Protein | D. radiodurans R1 | D. radiodurans R1 | D. radiodurans R1 | D. radiodurans R1 |
|---|---|---|---|---|
| (# of | chromosome 1 | chromosome 2 | circular plasmid 1 | megaplasmid 1 |
| residues) | (NC_001263) | (NC_001264) | (NC_000959) | (NC_000958) |
| KgtP | 36% identity (56 aa) | 30% identity (107 aa) | 32% identity (73 aa) | 58% identity (17 aa) |
| (432 aa) | “hypothetical protein | “sugar transporter | “hypothetical protein | “hypothetical protein |
| | DR_1056” | putative” | DR_C0021” | DR_B0098” |
| | (NP_294780.1) | (NP_285594.1) | (NP_051691.1) | (NP_051631.1) |
| YbjL | 24% identity (169 aa) | 33% identity (52 aa) | Not found | Not found |
| (561 aa) | “sodium/sulfate symporter | “transcriptional | | |
| | family protein” (NP_295134.1) | regulator” (NP_285659.1) | | |
| ProV | 35-41% identity (196-265 aa) | 27-38% identity (215-317 aa) | 36% identity (28 aa) | 32% identity (186-231 aa) |
| (400 aa) | “spermidine/putrescine ABC | “amino acid ABC | “transposase-related” | “iron ABC transporter |
| | transporter ATP-binding | transporter ATP-binding | (NP_051690.1) | ATP-binding protein” |
| | protein” (NP_295026.1), “ABC | protein” (NP_285461.1), | | (NP_051651.1), “ABC |
| | transporter ATP-binding | “phosphate ABC | | transporter, ATP-binding |
| | protein” (NP_295079.1, | transporter ATP-binding | | protein” (NP_051588.1) |
| | NP_295920.1, NP_293788.1), | protein” (NP_285484.1), | | |
| | “amino acid ABC transporter | “branched-chain amino | | |
| | ATP-binding protein” | acid ABC transporter | | |
| | (NP_295371.1), “sugar ABC | ATP-binding protein” | | |
| | transporter, ATP-binding | (NP_285584.1), “ABC | | |
| | protein” (NP_295876.1), | transporter ATP-binding | | |
| | “peptide ABC transporter | protein” (NP_285331.1, | | |
| | ATP-binding protein” | NP_285672.1), | | |
| | (NP_295290.1) | “urea/short-chain amide | | |
| | | ABC transporter | | |
| | | ATP-binding protein” | | |
| | | (NP_285647.1) | | |
| ProW | 26-31% identity (116-183 aa) | 30-35% identity (114-158 aa) | 29% identity (62 aa) | 28% identity (57 aa) |
| (354 aa) | “ABC transporter ATP-binding | “amino acid ABC | “transposase, putative” | “iron ABC transporter |
| | protein” (NP_294234.1), “ABC | transporter permease” | (NP_051699.1) | permease” |
| | transporter permease” | (NP_285462.1, | | (NP_051652.1) |
| | (NP_295919.1) | NP_285460.1) | | |
| ProX | Not found | 29% identity (68 aa) | 29% identity (41 aa) | 65% identity (17 aa) |
| (330 aa) | | “putative FAD-binding | “phosphoenolpyruvate | “hypothetical protein |
| | | dehydrogenase” | synthase-related protein” | DR_B0023” |
| | | (NP_285561.1) | (NP_051677.1) | (NP_051564.1) |
| ProQ | 31% identity (58 aa) | Not found | Not found | Not found |
| (232 aa) | “hypothetical protein | | | |
| | DR_2622” (NP_296341.1) | | | |
| CspE | 59% identity (64 aa) “CSD | 36-39% identity (31-36 aa) | 22-33% identity (45-50 aa) | Not found |
| (69 aa) | family cold shock protein” | “methyl-accepting | “hypothetical protein | |
| | (NP_294631.1) | chemotaxis-like protein” | DR_C0022” | |
| | | (NP_285676.1), “methyl- | (NP_051692.1), | |
| | | accepting chemotaxis | “nodulation protein- | |
| | | protein” (NP_285677.1) | related protein” | |
| | | | (NP_051705.1) | |
| RfaE | 26% identity (274 aa) | 33% identity (78 aa) | 30% identity (54 aa) | 26% identity (248 aa) |
| (477 aa) | “carbohydrate kinase” | “ribokinase” | “coenzyme PQQ synthesis | “1-phospho- |
| | (NP_296273.1) | (NP_285378.1) | protein, putative” | fructokinase” |
| | | | (NP_051702.1) | (NP_051610.1) |
| YfbP | Not found | 39% identity (61 aa) | 21% identity (85 aa) | Not found |
| (282 aa) | | “hypothetical protein | “oxidative cyclase, | |
| | | DR_A0109” | putative” | |
| | | (NP_285432.1) | (NP_051704.1) | |
| YfjM | Not found | Not found | Not found | Not found |
| (87 aa) | | | | |
| PstS | Not found | 44% identity (336 aa) | Not found | 36% identity (47 aa) |
| (346 aa) | | “phosphate ABC | | “hypothetical protein |
| | | transporter periplasmic | | DR_B0023” |
| | | phosphate-binding | | (NP_051564.1) |
| | | protein” (NP_285481.1) | | |
| Rph | 44% identity (215 aa) | 33% identity (62 aa) | Not found | 35% identity (31 aa) “iron- |
| (228 aa) | “ribonuclease PH” | “uroporphyrin-III C- | | chelator utilization |
| | (NP_295308.1) | methyltransferase/ | | protein” |
| | | uroporphyrinogen-III | | (NP_051560.1) |
| | | synthase (NP_285335.1) | | |
| RpoS | 42% identity (288 aa) “RNA | 30% identity (47 aa) | 34% identity (50 aa) | Not found |
| (330 aa) | polymerase sigma-A factor” | “hypothetical protein | “hypothetical protein | |
| | (NP_294640.1) | DR_A0192” | DR_C0027” | |
| | | (NP_285515.1) | (NP_051697.1) | |
| SspA | 24-29% identity (48-99 aa) | 35% identity (46 aa) “P49 | 24% identity (71 aa) | Not found |
| (212 aa) | “glutaredoxin” (NP_295808.1), | secreted protein” | “hypothetical protein | |
| | “hypothetical protein | (NP_285686.1) | DR_C0009” | |
| | DR_0390” (NP_294113.1) | | (NP_051682.1) | |
| Tdk | 27% identity (191 aa) | 24% identity (80 aa) | 27% identity (52 aa) | 38% Identity (50 aa) |
| (205 aa) | “thymidine kinase” | “urea/short-chain amide | “putative transposase” | “ABC transporter, |
| | (NP_295707.1) | ABC transporter | (NP_277100.1) | ATP-binding |
| | | periplasmic urea/short- | | protein” |
| | | chain amide-binding | | (NP_051588.1) |
| | | protein” (NP_285643.1) | | |
| UvrB | 56% identity (661 aa) | 33% identity (96 aa) | Not found | Not found |
| (673 aa) | “excinuclease ABC subunit B” | “hypothetical protein | | |
| | (NP_295996.1) | DR_A0131” | | |
| | | (NP_285455.1) | | |
| YcjG | 24-30% identity (285-306 aa) | Not found | Not found | 29% identity (201 aa) |
| (321 aa) | “chloromuconate | | | “N-acylamino acid |
| | cyclosisomerase” | | | racemase” |
| | (NP_295594.1), “N-acylamino | | | (NP_051613.1) |
| | acid racemase” | | | |
| | (NP_293770.1) | | | |
| YeaR | Not found | 37% identity (35 aa) | 29% identity (68 aa) | 30% identity (89 aa) |
| (119 aa) | | “exopolyphosphatase” | “nodulation protein- | “KdpD-related protein” |
| | | (NP_285509.1) | related protein” | (NP_051621.1) |
| | | | (NP_051705.1) | |
| SpoT | 38% identity (734 aa) “GTP | 30% identity (53 aa) | Not found | Not found |
| (702 aa) | pyrophosphokinase” | “long-chain-fatty-acid- | | |
| | (NP_295561.1) | CoA ligase” | | |
| | | (NP_296364.1) | | |
| PolB | 24-32% identity (65-135 aa) | 34% identity (65 aa) | 54% identity (13 aa) | 30% identity (69 aa) |
| (783 aa) | “excinuclease ABC subunit A” | “P49 secreted protein” | “hypothetical protein | “hypothetical protein |
| | (NP_295494.1), “hypothetical | (NP_285686.1) | DR_C0014” | DR_B0054” |
| | protein DR_2521” | | (NP_051686.1) | (NP_051592.1) |
| | (NP_296241.1) | | | |
| RpoB | 40-53% identity (in stretches, | 29% identity (63 aa) | 33% identity (40 aa) | 34% identity (44 aa) |
| (1342 aa) | 227-620 aa) “DNA-directed | “hypothetical protein | “modification methylase, | “hypothetical protein |
| | RNA polymerase subunit beta” | DR_A0017” | putative” | DR_B0144” |
| | (NP_ 294636.1) | (NP_285341.1) | (NP_277101.1) | (NP_051673.1) |
| RpoC | 45-58% identity (in stretches, | 37% identity (30 aa) | 48% identity (27 aa) | 32% identity (62 aa) |
| (1407 aa) | 156-937 aa) “DNA-directed | “succinate-semialdehyde | “putative transposase” | “hypothetical protein |
| | RNA polymerase subunit | dehydrogenase” | (NP_277100.1) | DR_B0013” |
| | beta'” (NP_294635.1) | (NP_285327.1) | | (NP_051556.1) |
| Rnt | 25% identity (192 aa) “DNA | 38% identity (37 aa) | 22% identity (79 aa) | Not found |
| (215 aa) | polymerase III subunit | “transcriptional | “hypothetical protein | |
| | epsilon” | regulator” | DR_C0027” | |
| | (NP_294580.1) | (NP_285659.1) | (NP_051697.1) | |
| SapC | 26-39% identity (215-230 aa) | 32% identity (248 aa) | Not found | 39% identity (23 aa) |
| (296 aa) | “peptide ABC transporter | “peptide ABC transporter | | “sensor histidine kinase, |
| | permease” (NP_294682.1, | permease” | | copper metabolism” |
| | NP_295292.1, NP_294088.1) | (NP_285531.1) | | (NP_051623.1) |
| PyrE | 31% identity (154 aa) “orotate | 32% identity (86 aa) | Not found | 38% identity (39 aa) |
| (213 aa) | phosphoribosyltransferase” | “serine protease” | | “hypothetical protein |
| | (NP_294170.1) | (NP_285606.1) | | DR_B0068” (NP_051604.1) |

So, in one aspect, there is provided a bacterial cell according to any one of the preceding aspects and embodiments, wherein each recited gene is instead (i) a gene encoding the corresponding (homolog or ortholog) protein in Table 3 or 4 (ii) a gene located at the corresponding locus, or (iii) both.

### Example 1

### Methods

### Screening for Tolerance in Wild-Type Cells

Escherichia coli K-12 MG1655 was grown overnight in M9 minimal medium+1% glucose and subcultured the following morning to an initial OD₆₀₀ of 0.05 in M9+1% glucose. Cells were grown to mid-exponential phase (OD₆₀₀ 0.7-1.0) and were back-diluted with fresh medium to an OD₆₀₀ of 0.7. The diluted cells were used to inoculate M9+1% glucose containing varying concentrations of glutaric acid or adipic acid which were neutralized to pH 7.0 with sodium hydroxide, and growth was measured in FlowerPlates in a Biolector microbioreactor system (m2p-labs) at 37° C. with 1000 rpm shaking. The culture volume in each well was 1.4 mL.

Adaptive Laboratory Evolution of Tolerant Strains

Based on the screening results, E. coli K-12 MG1655 was grown overnight in M9 minimal medium and 150 μL was transferred the next day into 8 tubes containing 15 mL of M9+1% glucose+20 g/L glutaric acid or 25 g/L adipic acid on a Tecan Evo robotic platform custom-designed for performing adaptive laboratory evolutions (ALE). Cells were cultured on a 37° C. heat block with stirring by magnetic stir bars. Culture OD₆₀₀ was monitored at times determined by a predictive custom script, and when the OD₆₀₀ reached approximately 0.3, 150 μL of culture was inoculated into a new tube with the same media concentration. Instrument downtime would occasionally result in cells overgrowing to saturation or an OD₆₀₀ greater than 0.3, and reinoculations were occasionally performed from cryogenic stocks of the population. When the growth rate was observed to substantially increase, the media concentration was changed. These concentration changes for glutaric acid were to 30 g/L, 40 g/L, and 45 g/L, and 47.5 g/L, while the changes adipic acid were to 35 g/L, 40 g/L, 45 g/L and 50 g/L. Approximately 100 μL of each population (8 per chemical) were plated on LB agar and incubated at 37° C. overnight.

Primary Screening of ALE Isolates

Five colonies from wild-type K-12 MG1655 and 10 individual colonies deriving from each population were inoculated into 300 μL M9+1% glucose in 96 well deepwell plates and incubated in a 300 rpm plate shaker at 37° C. The next day, cells were diluted 10× in M9+1% glucose and 30 μL was transferred into clear-bottomed 96 well half-deepwell plates (with rectangular wells) containing M9+1% glucose and M9+1% glucose+52.78 g/L glutaric acid or 55.56 g/L adipic acid, such that the final concentration of glutaric acid or adipic acid was 47.5 g/L or 50 g/L, respectively. In addition, cryogenic glycerol stocks of the overnight culture were saved in a 96 well plate format. Half deepwell plates were incubated at 37° C. with 225 rpm shaking in a Growth Profiler (Enzyscreen), with optical scans of the plates taken at 15 minute intervals. Green pixel values integrated over a 1 mm diameter circular area in each well were converted to OD₆₀₀ values using a previously determined calibration between OD₆₀₀ and green pixel values. Resulting growth curves were visually inspected for isolates exhibiting the most robust or unique growth patterns within each population. In general, it was attempted to select three isolates per population for further analysis, and all populations were represented in the resequenced isolates.

Secondary Screening of ALE Isolates

Selected isolates from the primary screen were restruck onto LB agar from the cryogenic stock made from the overnight culture plate for the primary screen. Five K-12 MG1655 colonies and three individual colonies from each isolate were inoculated as biological replicates into a new 96 well deepwell plate containing 300 μL of M9+1% glucose, and grown overnight as for the primary screen. The next day, a cryogenic stock and half deepwell plates containing M9+1% glucose with or without glutaric acid or adipic acid were inoculated using the plate of overnight cultures, and growth was measured as described for the primary screen. Resulting growth curves were visually inspected for isolates exhibiting robust and reproducible growth between replicates in high concentrations of glutaric acid or adipic acid.

Re-Sequencing of ALE Isolates

A total of 20 isolates were selected from the secondary screen for whole-genome resequencing. An individual colony was taken from the LB agar plates prepared following the primary screen, inoculated into 2 mL LB, and grown overnight at 37° C. in a 250 rpm shaker. The following morning, 0.5 mL of cells were transferred to microcentrifuge tubes and centrifuged at 16000×g for 2 minutes. The supernatant was removed and pellets were stored at −20° C. until further processing. Genomic DNA was extracted from thawed cell pellets using a PureLink genomic DNA extraction kit, with further concentration and purification performed by ethanol precipitation. To generate libraries for sequencing, the Illumina TruSeq Nano kit was used according to the manufacturers' directions using an input quantity of 200 ng of genomic DNA from each isolate. Sequencing was performed on an Illumina MiSeq sequencer, with a minimum 20×average genomic coverage ensured for each isolate based on the number of reads. Fastq output files were analyzed for variants compared to the K-12 MG1655 reference genome (accession number NC_000913.3) using breseq (www-adress barricklab.org/twiki/bin/view/Lab/ToolsBacterialGenomeResequencing).

Sole Carbon Source Plate Growth Assay

M9 agar plates lacking glucose and instead containing 10 g/L of glutaric acid or 10 g/L of adipic acid (both neutralized to pH 7.0 with sodium hydroxide) were prepared, and strains were struck onto wedges of the plate from a colony on an LB plate. Plates were incubated for up to 4 weeks at 37° C.

Construction of Gene Knockouts

Probable important losses-of-function were determined by identifying genes across all isolates that harboured mutations, especially those occurring in multiple populations, and by the presence of at least one mutation that either generated a premature stop codon, a frameshift mutation, or the presence of an insertion element sequence within the gene. For those genes, the corresponding knockout strain from the Keio collection of single knockout mutants (where each gene is replaced with a cassette consisting of a kanamycin resistance gene flanked by FRT sites) was used as a donor strain for P1vir phage transduction. Briefly, the Keio strain was grown to early exponential phase in LB+5 mM CaCl₂ and 80 μL of a P1vir stock raised on K-12 MG1655 was added. After significant lysis was observed after 1.5 to 2 hours, the lysate was filter-sterilized to remove cells and stored at 4° C. Strain K-12 MG1655 was grown overnight in LB+5 mM CaCl₂) and 100 μL of the overnight culture was mixed with 100 μL of the P1vir lysate of the Keio collection mutant, and the mixture was incubated at 37° C. without shaking for 20 minutes. The entire mixture was then plated on LB agar containing 1.25 mM sodium pyrophosphate as a chelating agent and 25 μg/mL kanamycin. One colony was then restruck on LB+1.25 mM Na₂P₄O₇+25 μg/mL kanamycin plate and analyzed for presence of the Keio cassette in place of the wild-type gene by colony PCR. When further knockouts were constructed in the same strain, the Keio cassette was flipped out to generate a scar sequence such that Kan^{R} marker could be recycled. This was performed by transforming with pCP20, which constitutively expresses a flippase recombinase, and plating cells on LB agar+100 μg/mL ampicillin and incubating at 30° C. The next day, one or more colonies was tested by colony PCR for loss of the Keio cassette, and successful mutants were then cured of pCP20 by elevated temperature curing at 40° C. Strains were verified to be cured of plasmid by plating on LB agar+100 μg/mL ampicillin and incubation at 30° C. P1vir transductions were then performed using these mutant strains as recipients.

### Biolector Growth Screening of Evolved Isolates and Reconstructed Mutants

Biological triplicate cultures of each strain were grown to saturation overnight in 96 well deepwell plates containing 300 μL M9+1% glucose. The next day, cells were diluted 1:10 in deionized water in a clear 96 well plate and the OD₆₀₀ was measured on a BioTek plate reader. 48 well FlowerPlates containing a final volume of 1.4 mL of M9+1/o glucose (plus relevant chemical) were inoculated to OD₆₀₀ 0.03 (with plate reader pathlength, 200 μL volume) with the overnight culture and sealed with Breathseal film. Light backscatter intensity was monitored in a Biolector microbioreactor system at 37° C. with 1000 rpm shaking.

Keio Collection Screening for Loss-of-Function Mutations

For primary screening, Keio collection mutants were inoculated directly from a cryogenic stock of the Keio collection into 300 μL LB medium containing 25 μg/mL kanamycin in 96 well deepwell plates and grown at 37° C. with 300 rpm shaking overnight. The Keio background strain, BW25113, was also inoculated into wells of this plate as a control. A cryogenic stock was made from each plate, and the cryogenic stock was replica plated into another 96 well deepwell plate containing 300 μL M9+1% glucose and grown overnight. The next day, cells were inoculated 1:100 into clear bottomed 96 well half-deepwell plates containing M9+1% glucose plus 40 g/L and 47.5 g/L putrescine, or 45 and 50 g/L adipic acid, and cultivated in a Growth Profiler as previously described for screening of ALE isolates.

As a secondary screen, promising Keio collection mutants were struck on LB+25 μg/mL kanamycin from the cryogenic stock plate prepared during primary screening above and biological triplicate colonies were inoculated into a 96 well deepwell plate containing 300 μL M9+1% glucose. The next day, cells were inoculated into plates for cultivation on the Growth Profiler as described above.

### Cross-Compound Tolerance Screening

96 well deepwell plates containing 300 μL of M9+1% glucose were inoculated directly from cryogenic stocks made from precultures for the secondary screening of ALE isolates and were grown overnight at 37° C. with 300 rpm shaking. The next day, cells were diluted 1:100 into 96 well half-deepwell plates containing the following final concentrations of each chemical in M9+1% glucose:

| | | |
|---|---|---|
| butanol | 1.4% | v/v |
| glutarate | 40 | g/L |
| p-coumarate | 7.5 | g/L |
| putrescine | 32 | g/L |
| HMDA | 32 | g/L |
| adipate | 45 | g/L |
| isobutyrate | 7.5 | g/L |
| hexanoate | 3 | g/L |
| octanoate | 8 | g/L |
| 2,3-butanediol | 6% | v/v |
| 1,2-propanediol | 6% | v/v |

| | |
|---|---|
| sodium chloride | 0.6M |

Plates were cultivated in a Growth Profiler for 48 hours as described for screening of ALE isolates. Green pixel integrated values from each well were converted to OD₆₀₀ values using a calibration curve and the resulting OD₆₀₀ vs. elapsed time data was processed using custom scripts to determine the time required for each culture to reach an OD of 1.0 (t_{OD1}). This value is a combined measure of growth rate and lag time in each culture. The median value was taken for biological triplicates of each isolate and was normalized to the median t_{OD1} for K-12 MG1655 controls (5 replicates). The ratio of t_{OD1(evolved)}/t_{OD1(wild-type)} is presented.

### Results

### Wild-Type Tolerance to Diacids

The maximum measured concentration of glutaric acid at which exponentially growing K-12 MG1655 can grow was found to be 50 g/L with severe inhibition (Table 5). Increasing inhibition of growth was observed from 10 to 50 g/L.

TABLE 5
Growth of K-12 MG1655 in varying concentrations of
glutaric acid (neutralized with sodium hydroxide).

| | |
|---|---|
| Mean | std. error |

| glutaric acid | μ | t_{lag} | μ | t_{lag} |
|---|---|---|---|---|
| (g/L) | (h⁻¹) | (h) | (h⁻¹) | (h) |
| 0 | 0.661 | 0.7 | 0.011 | 0.1 |
| 10 | 0.526 | 2.0 | 0.019 | 0.1 |
| 20 | 0.377 | 1.9 | 0.006 | 0.3 |
| 30 | 0.234 | 0.6 | 0.034 | 1.1 |
| 40 | 0.132 | 0.7 | 0.028 | 1.7 |
| 50 | 0.096 | 15.9 | 0.062 | 0.8 |
| 75 | 0.000 | — | 0.000 | — |

The maximum measured concentration of adipic acid at which exponentially growing K-12 MG1655 can grow was found to be 75 g/L with an extensive lag phase of 27 hours (Table 6). Growth rates dropped sharply as a function of concentration between 10 and 50 g/L.

TABLE 6
Growth of K-12 MG1655 in varying concentrations of
adipic acid (neutralized with sodium hydroxide).

| | |
|---|---|
| Mean | std. error |

| adipic acid | μ | t_{lag} | μ | t_{lag} |
|---|---|---|---|---|
| (g/L) | (h⁻¹) | (h) | (h⁻¹) | (h) |
| 0 | 0.653 | 0.6 | 0.007 | 0.0 |
| 10 | 0.575 | 1.0 | 0.018 | 0.1 |
| 20 | 0.494 | 1.3 | 0.017 | 0.2 |
| 30 | 0.367 | 1.8 | 0.030 | 0.7 |
| 40 | 0.263 | 4.0 | 0.012 | 0.4 |
| 50 | 0.120 | 2.4 | 0.009 | 1.6 |
| 75 | 0.203 | 27.2 | 0.082 | 0.6 |
| 100 | 0.000 | — | 0.000 | — |

Growth was also tested in pimelic acid (C₇) and sebacic acid (C₁₀) (Table 7). Robust growth was still observed in pimelic acid at 45 g/L, however inhibition was observed as a function of increasing concentration. Sebacic acid was more toxic, with nearly no growth detected above 40 g/L concentration.

TABLE 7
Growth of K-12 MG1655 in varying concentrations of pimelic
and sebacic acids (neutralized with sodium hydroxide).

| | pimelic acid | | | | sebacic acid | | | |
|---|---|---|---|---|---|---|---|---|
| | mean | | std. error | | Mean | | std. error | |
| concentration | μ | t_{lag} | μ | t_{lag} | μ | t_{lag} | μ | t_{lag} |
| (g/L) | (h⁻¹) | (h) | (h⁻¹) | (h) | (h⁻¹) | (h) | (h⁻¹) | (h) |
| 0 | 0.650 | 0.5 | 0.027 | 0.2 | 0.668 | 0.5 | 0.016 | 0.0 |
| 10 | 0.694 | 1.9 | 0.021 | 0.2 | 0.707 | 1.9 | 0.048 | 0.2 |
| 20 | 0.656 | 2.4 | 0.022 | 0.1 | 0.578 | 2.7 | 0.057 | 0.3 |
| 25 | 0.572 | 2.5 | 0.021 | 0.2 | 0.413 | 3.3 | 0.003 | 0.2 |
| 30 | 0.560 | 3.3 | 0.021 | 0.2 | 0.386 | 5.7 | 0.054 | 0.2 |
| 35 | 0.534 | 4.1 | 0.006 | 0.0 | 0.306 | 10.8 | 0.026 | 0.4 |
| 40 | 0.439 | 4.3 | 0.008 | 0.3 | 0.222 | 22.5 | 0.030 | 0.6 |
| 45 | 0.280 | 4.6 | 0.007 | 0.3 | 0.051 | 50.6 | 0.020 | 3.4 |

Aiming for a starting growth rate between 0.3 to 0.4 h⁻¹, it was decided to begin evolutions at a concentration of 20 g/L glutaric acid and 25 g/L adipic acid.

### Resequencing of Tolerant Isolates

Variants detected in glutaric and adipic acid evolved strains are presented in Tables 8 and 9. Each strain name corresponds to the chemical the strain was isolated from, the population the strain was isolated from, and the original number of the strain assigned during primary screening (e.g. GLUT1-3 is a glutaric acid-evolved strain isolated from population 1). In each table, strains are arranged such that all that were isolated from the same population are presented in the same rows. Strains with an asterisk (*) following their name are hypermutator strains, and only the mutation identified that can be associated with generating the hypermutator phenotype (here only in mutS or mutT in 1 isolate from 1 glutaric acid population and all isolates from 1 adipic acid population) and those mutations that are shared with other mutations in the same gene in other strains are shown.

Mutations that occur independently across multiple populations, or that appear fixed in a highly variable population are likely causative and of highest interest. For glutaric acid, these include mutations in kgtP or its promoter region (24 out of 24 isolates), spoT (all isolates except in population GLUT8), rpoC (9 isolates in 5 populations), proV (6 isolates in 3 populations) and proX (2 isolates in 1 population), rnt (5 isolates in 3 populations), nagC and nagA (4 isolates in 2 populations). In place of mutations in spoT, a coding mutation in polB was found in all 3 isolates of population GLUT8. Mutations in rpoB, encoding another subunit of RNA polymerase in addition to rpoC, were found in all 3 isolates from population GLUT5. Of these mutations, those of kgtP, proV and proX, and nagC are likely loss-of-function mutations, due to the presence of frameshift mutations, premature stop codons, or IS element insertions in at least one population of individual isolate that possesses mutations in that gene. Other mutations are likely gain-of-function or weakening of function, for example coding mutations in genes encoding subunits of RNA polymerase (RpoC and RpoB), SpoT (plus an in-frame deletion in one population), and PolB.

For adipic acid, mutated genes that occurred across multiple populations included those in kgtP (19 out of 19 isolates), ybjL (12 isolates in 6 populations), proV or its promoter region (11 isolates in 5 populations; plus 3 isolates in 1 population that possessed a large deletion spanning proV, proX, and proW plus other neighboring genes), sspA (7 isolates in 4 populations; also found in 1 isolate from glutaric acid), the intergenic region between pyrE and rph (6 isolates in 3 populations), nagC (5 isolates in 2 populations), yicC (5 isolates in 2 populations), spoT (4 isolates in 2 populations), and pstS or its promoter region (4 isolates in 2 populations). Notably lacking were mutations in any subunit of RNA polymerase. Of these mutations, those of kgtP, ybjL, proV, sspA, and nagC are likely loss-of-function mutations, due to the presence of frameshift mutations, premature stop codons, or IS element insertions in at least one population of individual isolate that possesses mutations in that gene. Coding mutations in SpoT are likely gain-of-function or weakening of function, as for glutaric acid. Mutations in sspA are either coding SNPs or an in-frame (21 bp) deletion, therefore it is unclear whether this mutation is a loss-of-function. PstS is one subunit of a transporter complex complex PstBACS which is involved in the import of inorganic phosphate under phosphate starvation conditions.

TABLE 8
Variants detected in glutaric acid-evolved isolates

| coordinate | gene | change | coordinate | gene | change | coordinate | gene | change |
|---|---|---|---|---|---|---|---|---|
| GLUT1-3 | | | GLUT1-9 | | | GLUT1-10 | | |
| 1347104 | rnb | C603* (G→T) | 1347104 | rnb | C603* (G→T) | 2725668 | kgtP | 1 bp deletion |
| 1630841 | ydfl | V25F (C→A) | 2725668 | kgtP | 1 bp deletion | 2726161 | [rrfG] | 4703 bp deletion |
| | | | | | | | [rrsG] | |
| 2725668 | kgtP | 1 bp deletion | 2726129 | [rrfG] | 4742 bp deletion | 2805532 | proV | 1 bp insertion (→T) |
| | | | | [rrsG] | | | | |
| 2726147 | [rrfG] | 4703 bp deletion | 3823664 | spoT | V422A (T→C) | 3328463 | greA | IS4 element insertion |
| | [rrsG] | | | | | | | |
| 3823664 | spoT | V422A (T→C) | 4186605 | rpoC | H419P (A→C) | 3377214 | sspA | 21 bp amplification |
| | | | | | | | | (X2) |
| 4186605 | rpoC | H419P (A→C) | | | | 3522182 | hofM | noncoding SNP |
| | | | | | | | | (C→A) |
| | | | | | | 3751884 | yiaT/yiaU | IS5 element insertion |
| | | | | | | 3823664 | spoT | V422A (T→C) |
| GLUT2-1 | | | GLUT2-9 | | | GLUT2-10 | | |
| 481075 | tomB/acrB | noncoding SNP | 1347882 | rnb | 1 bp deletion | 1654069 | rspA | T358S (G→C) |
| | | (A→G) | | | | | | |
| 2725642 | kgtP | 1 bp deletion | 2725642 | kgtP | 1 bp deletion | 2725642 | kgtP | 1 bp deletion |
| 2804858 | proV | 13 bp deletion | 3823751 | spoT | A451D (C→A) | 2804858 | proV | 13 bp deletion |
| 3636414 | ygjP | D152A (A→C) | | | | 3823751 | spoT | A451D (C→A) |
| 3823751 | spoT | A451D (C→A) | | | | 4186605 | rpoC | H419P(A→C) |
| GLUT3-5 | | | GLUT3-7 | | | GLUT3-9 | | |
| 2724971 | kgtP | F259C (A→C) | 2724971 | kgtP | F259C (A→C) | 318484 | ykgl | noncoding SNP |
| | | | | | | | | (C→T) |
| 3823770 | spoT | W457C (G→T) | 3823770 | spoT | W457C (G→T) | 996768 | ssuA | noncoding SNP |
| | | | | | | | | (G→A) |
| 4186605 | rpoC | H419P (A→C) | 4186605 | rpoC | H419P (A→C) | 1728882 | rnt | Q179P (A→C) |
| | | | | | | 2725518 | kgtP | 1 bp insertion (→C) |
| | | | | | | 3823759 | spoT | N454H (A→C) |
| | | | | | | 3969048 | wzzE | 1 bp insertion (→G) |
| GLUT4-1 | | | GLUT4-4 | | | GLUT4-10 | | |
| 2724611 | kgtP | A379V(G→A) | 2390019 | yfbP/nuoN | G→A | 2724971 | kgtP | F259C (A→C) |
| 2807193 | proX | 8 bp deletion | 2724611 | kgtP | A379V (G→A) | 2788702 | ygaQ/csiD | IS5 element insertion |
| 3824137 | spoT | D580Y (G→T) | 2807193 | proX | 8 bp deletion | 3823751 | spoT | A451V (C→T) |
| | | | 3195220 | ibsE/rfaE | IS186 element insertion | 4186605 | rpoC | H419P (A→C) |
| | | | 3824137 | spoT | D580Y (G→T) | | | |
| GLUT5-4 | | | GLUT5-5 | | | GLUT5-9 | | |
| 303119 | yagU | 1 bp deletion | 2725374 | kgtP | 1 bp deletion | 2725374 | kgtP | 1 bp deletion |
| 2725374 | kgtP | 1 bp deletion | 3377491 | sspA/rpsI | IS2 element insertion | 3377491 | sspA/rpsI | IS2 element insertion |
| 3377359 | sspA | 18 bp deletion | 3823106 | spoT | R236L (G→T) | 3823106 | spoT | R236L (G→T) |
| 3823106 | spoT | R236L (G→T) | 4181852 | rpoB | K203T (A→C) | 4181852 | rpoB | K203T (A→C) |
| 4181852 | rpoB | K203T (A→C) | 4451123 | ytfR | noncoding SNP | 4451123 | ytfR | noncoding SNP |
| | | | | | (C→A) | | | (C→A) |
| 4451123 | ytfR | noncoding SNP | | | | | | |
| | | (C→A) | | | | | | |
| GLUT6-4 | | | GLUT6-5 | | | GLUT6-10 | | |
| 657215 | pagP/cspE | 25 bp deletion | 657215 | pagP/cspE | 25 bp deletion | 1728425 | rnt | A27T (G→A) |
| 700680 | nagC | IS1 element insertion | 700680 | nagC | IS1 element insertion | 2672970 | hcaD | 6 bp deletion |
| 2725370 | kgtP | L126* (A→C) | 2725370 | kgtP | L126* (A→C) | 2724725 | kgtP | 6 bp insertion |
| | | | | | | | | (→CAAAAG) |
| 2765412 | yfjL/yfjM | 8 bp deletion | 2765412 | yfjL/yfjM | 8 bp deletion | 3823139 | spoT | M247K (T→A) |
| 3823105 | spoT | R236S (C→A) | 3823105 | spoT | R236S (C→A) | | | |
| GLUT7-2 | | | GLUT7-6 | | | GLUT7-7* | | |
| 701396 | nagC | Q67* (G→A) | 1636300 | ydfJ | T→G | 701377 | nagC | 1 bp deletion |
| 1728926 | rnt | F194L (T→C) | 1728926 | rnt | F194L (T→C) | 1728884 | rnt | A180T (G→A) |
| 2724848 | kgtP | G300V (C→A) | 2724848 | kgtP | G300V (C→A) | 2725668 | kgtP | 1 bp deletion |
| 3824201 | spoT | 6 bp detection | 3824201 | spoT | 6 bp deletion | 2859432 | mutS | noncoding SNP |
| | | | | | | | | (C→T) |
| | | | | | | 3823724 | spoT | T442I (C→T) |
| GLUT8-5 | | | GLUT8-6 | | | GLUT8-9 | | |
| 64352 | polB | R477G (G→C) | 64352 | polB | R477G (G→C) | 64352 | polB | R477G (G→C) |
| 2725818 | kgtP/rrfG | noncoding SNP | 702331 | nagA | 1 bp insertion (→T) | 1354284 | sapC | G79W (C→A) |
| | | (A→G) | | | | | | |
| 2804858 | proV | 13 bp deletion | 1354284 | sapC | G79W (C→A) | 1907448 | yobF | IS5 element insertion |
| 2810987 | mprA | IS1 element insertion | 2725232 | kgtP | G172V (C→A) | 2725208 | kgtP | 9 bp deletion |
| 4185540 | rpoC | P64L (C→T) | 2804858 | proV | 13 bp deletion | 2804921 | proV | 1 bp deletion |
| | | | 4185540 | ropC | P64L (C→T) | 2927703 | ygdH/sdaC | intergenic SNP |
| | | | | | | | | (G→A) |
| | | | | | | 4185540 | rpoC | P64L (C→T) |

TABLE 9
Variants detected in adipic acid-evolved isolates

| coordinate | gene | change | coordinate | gene | change | coordinate | gene | change |
|---|---|---|---|---|---|---|---|---|
| ADIP1-1 | | | ADIP1-9 | | | | | |
| 814029 | uvrB | D168E (T→G) | 546309 | allD | A19T (C→T) | | | |
| 889562 | ybjL | 1 bp deletion | 702405 | nagA | 36 bp amplification | | | |
| | | | | | (X2) | | | |
| 2725207 | kgtP | IS1 element | 889562 | ybjL | 1 bp deletion | | | |
| | | insertion | | | | | | |
| 2804648 | nrdF/proV | 38 bp deletion | 2530235 | ligA/ZipA | noncoding SNP | | | |
| | | | | | (G→T) | | | |
| 3377068 | sspA | 21 bp deletion | 2725207 | kgtP | IS1 element | | | |
| | | | | | insertion | | | |
| 3815823 | pyrE/rph | noncoding SNP | 2804648 | nrdF/proV | 38 bp deletion | | | |
| | | (C→A) | | | | | | |
| 3816848 | yicC | T58M (C→T) | 3377068 | sspA | 21 bp deletion | | | |
| 4294366 | nrfG/gltP | noncoding SNP | 3815823 | pyrE/rph | noncoding SNP | | | |
| | | (A→T) | | | (C→A) | | | |
| | | | 3816848 | yicC | T58M (C→T) | | | |
| | | | 4294366 | nrfG/gltP | noncoding SNP | | | |
| | | | | | (A→T) | | | |
| ADIP2-5 | | | ADIP2-6 | | | ADIP2-10 | | |
| 362830 | lacY | C117F (C→A) | 362830 | lacY | C117F (C→A) | 700628 | nagC | IS5 element |
| | | | | | | | | insertion |
| 700529 | nagC | IS1 element | 2725613 | kgtP | S45L (G→A) | 1530007 | ydcD | S29* (C→A) |
| | | insertion | | | | | | |
| 2725613 | kgtP | S45L (G→A) | 2798606 | alaE-ygaY | 10942 bp deletion | 2725613 | kgtP | S45L (G→A) |
| 2798606 | alaE-ygaY | 10942 bp deletion | 3815883 | rph | 2 bp deletion | 2798606 | alaE-ygaY | 10942 bp deletion |
| 3815883 | rph | 2 bp deletion | | | | 3815809 | pyrE/rph | 1 bp deletion |
| ADIP3-2 | | | ADIP3-4 | | | ADIP3-8 | | |
| 889534 | ybjL | IS5 element | 233954 | mltD | 2 bp insertion | 889534 | ybjL | IS5 element |
| | | insertion | | | (→TG) | | | insertion |
| 2614996 | yfgO | noncoding SNP | 889534 | ybjL | IS5 element | 2725207 | kgtP | IS1 element |
| | | (A→G) | | | insertion | | | insertion |
| 2725207 | kgtP | IS1 element | 1879829 | yeaR | IS186 element | 2804648 | nrdF/proV | 38 bp deletion |
| | | insertion | | | insertion | | | |
| 2804648 | nrdF/proV | 38 bp deletion | 2725207 | kgtP | IS1 element | 3377068 | sspA | 21 bp deletion |
| | | | | | insertion | | | |
| 3377068 | sspA | 21 bp deletion | 2804648 | nrdF/proV | 38 bp deletion | 3815823 | pyrE/rph | noncoding SNP |
| | | | | | | | | (C→A) |
| 3815823 | pyrE/rph | noncoding SNP | 3377068 | sspA | 21 bp deletion | 3816848 | yicC | T58M (C→T) |
| | | (C→A) | | | | | | |
| 3816848 | yicC | T58M (C→T) | 3633911 | yhiL | IS5 element | | | |
| | | | | | insertion | | | |
| | | | 3815823 | pyrE/rph | noncoding SNP | | | |
| | | | | | (A→C) | | | |
| | | | 3816848 | yicC | T58M (C→T) | | | |
| ADIP4-8 | | | | | | | | |
| 702405 | nagA | 36 bp amplification | | | | | | |
| | | (X2) | | | | | | |
| 889488 | ybjL | IS1 element | | | | | | |
| | | insertion | | | | | | |
| 1728708 | rnt | N121S(A→G) | | | | | | |
| 2675452 | yphC | P104A(G→C) | | | | | | |
| 2693818 | purL | V576L(C→G) | | | | | | |
| 2701175 | pdxJ | noncoding SNP | | | | | | |
| | | (G→C) | | | | | | |
| 2713302 | srmB | R136L(G→T) | | | | | | |
| 2724590 | kgtP | G386D(C→T) | | | | | | |
| 3548179 | malQ | A631D(G→T) | | | | | | |
| 4130167 | metL | 462 bp deletion | | | | | | |
| ADIP5-2* | | | ADIP5-6* | | | | | |
| 111305 | mutT | E88* (G→T) | 111305 | mutT | E88* (G→T) | | | |
| 888480 | ybjL | F447C (A→C) | 888948 | ybjL | E291A (T→G) | | | |
| 2724576 | kgtP | Y391D (A→C) | 2615642 | yfgO | noncoding SNP | | | |
| | | | | | (T→G) | | | |
| 2805674 | proV | L287* (T→G) | 2724576 | kgtP | Y391D (A→C) | | | |
| 3549583 | malQ | D163A (T→G) | 2805674 | proV | L287* (T→G) | | | |
| | | | 3377387 | sspA | T12P (T→G) | | | |
| ADIP6-3 | | | ADIP6-9 | | | ADIP6-10 | | |
| 700928 | nagC | G223* (C→A) | 700928 | nagC | G223* (C→A) | 700928 | nagC | G223* (C→A) |
| 889534 | ybjL | IS5 element | 889534 | ybjL | IS5 element | 889534 | ybjL | IS5 element |
| | | insertion | | | insertion | | | insertion |
| 1915297 | proQ | R80C (G→A) | 1196319 | icd | noncoding SNP | 1915297 | proQ | R80C (G→A) |
| | | | | | (C→A) | | | |
| 2725155 | kgtP | R198S (G→T) | 1389396 | ycjG | L156P (T→C) | 2725155 | kgtP | R198S (G→T) |
| 2804858 | proV | 13 bp deletion | 1915297 | proQ | R80C (G→A) | 2805493 | proV | E227* (G→T) |
| 3823700 | spoT | S434L (C→T) | 2725155 | kgtP | R198S (G→T) | 3823700 | spoT | S434L (C→T) |
| 4019173 | ubiE | K107E (A→G) | 2804831 | proV | 7 bp deletion | | | |
| | | | 3823700 | spoT | S434L (C→T) | | | |
| ADIP7-2 | | | ADIP7-5 | | | | | |
| 293574 | yagL | noncoding SNP | 889540 | ybjL | IS5 element | | | |
| | | (A→G) | | | insertion | | | |
| 1293038 | hns/tdk | IS1 element | 2725642 | kgtP | 1 bp deletion | | | |
| | | insertion | | | | | | |
| 1598223 | yneO/lsrK | IS5 element | 2804858 | proV | 13 bp deletion | | | |
| | | insertion | | | | | | |
| 2725329 | kgtP | G140* (C→A) | 3377240 | sspA | T61P (T→G) | | | |
| 2867354 | rpoS | 9 bp deletion | 4490689 | idnR | IS1 element | | | |
| | | | | | insertion | | | |
| 3910996 | pstS | 4 bp insertion | | | | | | |
| | | (→CTTT) | | | | | | |
| ADIP8-3 | | | ADIP8-7 | | | ADIP8-10 | | |
| 1293015 | hns/tdk | IS1 element | 1293015 | hns/tdk | IS1 element | 280003 | insl1 | IS5 element |
| | | insertion | | | insertion | | | insertion |
| 2192447 | yehD/yehE | 1 bp deletion | 2192447 | yehD/yehE | 1 bp deletion | 2192447 | yehD/yehE | 1 bp deletion |
| 2724588 | kgtP | G387S (C→T) | 2724588 | kgtP | G387S (C→T) | 2724588 | kgtP | G387S (C→T) |
| 3911563 | pstS/glmS | IS1 element | 3911563 | pstS/glmS | IS1 element | 3911563 | pstS/glmS | IS1 element |
| | | insertion | | | insertion | | | insertion |

### Characterization of Selected Isolates

Each re-sequenced isolate was characterized using the Biolector system for growth at the screening concentration of chemical (47.5 g/L glutaric acid or 50 g/L adipic acid) in biological triplicates. The average growth rates with standard errors for the three replicates are shown in Tables 10 and 11.

Variations in growth behavior amongst evolved isolates can be noted. Better growing strains are defined by both the slope of the curve (higher growth rate) and at what time the cultures begin growing (reduced lag time). Some isolates exhibit poorer improvements in growth rates (e.g. ADIP7-2 and ADIP8 isolates) but especially reduced lag times. The phenotype to genotype relationship infers mutations that are of highest interest and those that are not of interest. For example, GLUT2-10 was the best performing isolate from population GLUT2, indicating that either the RpoC-H419P and/or RpsA-T358S mutations are causative for higher growth rate, or the lack of other mutations found in the other two isolates is beneficial. Another example of this would, for example, be when multiple isolates from one population are growing nearly identically (e.g. GLUT4-1 and GLUT4-4). This indicates that any differences in mutations between these two isolates are not important for tolerance, in this case the intergenic mutations between yfbP and nuoN, and between ibsE and rfaE, found in GLUT4-4.

TABLE 10
Growth rates and lag times of re-sequenced glutaric
acid evolved isolates in M9 + 47.5 g/L glutaric
acid (neutralized with sodium hydroxide).

| | |
|---|---|
| mean | std. error |

| | μ | t_{lag} | μ | t_{lag} |
|---|---|---|---|---|
| strain | (h⁻¹) | (h) | (h⁻¹) | (h) |
| MG1655 | 0.103 | 10.7 | 0.020 | 4.7 |
| GLUT1-3 | 0.304 | 5.1 | 0.012 | 1.8 |
| GLUT1-9 | 0.319 | 6.4 | 0.012 | 0.7 |
| GLUT1-10 | 0.458 | 11.3 | 0.085 | 0.1 |
| GLUT2-1 | 0.276 | 6.8 | 0.077 | 0.6 |
| GLUT2-9 | 0.298 | 6.5 | 0.014 | 1.5 |
| GLUT2-10 | 0.378 | 6.5 | 0.051 | 0.5 |
| GLUT3-5 | 0.284 | 5.6 | 0.021 | 2.1 |
| GLUT3-7 | 0.277 | 5.6 | 0.044 | 3.1 |
| GLUT3-9 | 0.324 | 8.3 | 0.014 | 3.8 |
| GLUT4-1 | 0.279 | 6.2 | 0.007 | 1.1 |
| GLUT4-4 | 0.287 | 6.6 | 0.015 | 1.1 |
| GLUT4-10 | 0.297 | 6.9 | 0.020 | 2.8 |
| GLUT5-4 | 0.438 | 17.0 | 0.098 | 18.0 |
| GLUT5-5 | 0.349 | 14.5 | 0.054 | 13.8 |
| GLUT6-4 | 0.277 | 5.9 | 0.013 | 0.7 |
| GLUT6-5 | 0.300 | 6.5 | 0.025 | 0.7 |
| GLUT6-10 | 0.341 | 6.7 | 0.093 | 0.9 |
| GLUT7-2 | 0.318 | 6.6 | 0.027 | 0.2 |
| GLUT7-6 | 0.318 | 7.1 | 0.010 | 1.2 |
| GLUT7-7 | 0.283 | 7.8 | 0.023 | 0.8 |
| GLUT8-5 | 0.322 | 6.2 | 0.018 | 0.9 |
| GLUT8-6 | 0.347 | 7.1 | 0.013 | 0.9 |
| GLUT8-9 | 0.345 | 7.4 | 0.042 | 2.4 |

TABLE 11
Growth rates and lag times of re-sequenced adipic
acid evolved isolates in M9 + 50 g/L adipic
acid (neutralized with sodium hydroxide).

| | |
|---|---|
| mean | std. error |

| | μ | t_{lag} | μ | t_{lag} |
|---|---|---|---|---|
| strain | (h⁻¹) | (h) | (h⁻¹) | (h) |
| MG1655-1 | 0.133 | 21.4 | 0.017 | 2.1 |
| ADIP1-1 | 0.315 | 16.6 | 0.037 | 5.6 |
| ADIP1-9 | 0.274 | 5.8 | 0.013 | 1.4 |
| ADIP2-5 | 0.332 | 20.1 | 0.019 | 2.4 |
| ADIP2-6 | 0.337 | 22.1 | 0.038 | 2.0 |
| ADIP2-10 | 0.326 | 20.8 | 0.007 | 1.6 |
| ADIP3-2 | 0.295 | 16.6 | 0.011 | 4.1 |
| ADIP3-4 | 0.305 | 23.3 | 0.017 | 11.5 |
| ADIP3-8 | 0.295 | 20.1 | 0.032 | 4.7 |
| ADIP4-8 | 0.332 | 6.8 | 0.022 | 0.7 |
| ADIP5-2 | 0.401 | 4.7 | 0.015 | 1.3 |
| ADIP5-6 | 0.370 | 14.4 | 0.130 | 7.9 |
| ADIP6-3 | 0.280 | 6.5 | 0.003 | 0.4 |
| ADIP6-9 | 0.279 | 6.8 | 0.014 | 0.5 |
| ADIP6-10 | 0.284 | 7.1 | 0.023 | 0.2 |
| ADIP7-2 | 0.189 | 7.6 | 0.024 | 4.4 |
| ADIP7-5 | 0.292 | 19.3 | 0.006 | 12.7 |
| ADIP8-3 | 0.201 | 6.0 | 0.011 | 1.4 |
| ADIP8-7 | 0.206 | 7.2 | 0.011 | 0.5 |
| ADIP8-10 | 0.200 | 6.8 | 0.016 | 1.9 |

Sole Carbon Source Plate Growth Assay Wild-type, glutaric acid, and adipic acid evolved strains were struck on M9 agar containing glutarate or adipate as a sole carbon source. No growth was observed on adipic acid plates, indicating that E. coli cannot utilize adipic acid as a sole carbon source. Robust but very slow growth of wild-type K-12 MG1655 was observed after a few weeks (Table 12), indicating the ability of E. coli to use glutarate as a sole carbon source, almost certainly through promiscuous activity of pathway enzymes (because glutarate is not a natural metabolite in E. coli). Growth on this compound as a sole carbon source has not been previously reported in the literature. Evolved isolates generally could not grow on glutaric acid, with the exception of weak growth exhibited by GLUT8-9. The only common genetic feature between all evolved isolates relative to the wild-type are probable loss-of-function mutations in kgtP, implicating KgtP, an α-ketoglutarate importer, as being a direct importer for glutarate. GLUT8-9 notably features an in-frame 9 bp deletion in kgtP that is unique among all the isolates. Without being limited to theory, this mutation may result in a reduced activity of KgtP, rather than a full loss-of-function.

TABLE 12
Qualitative growth score of glutarate-evolved isolates
on M9 agar plates containing 10 g/L glutaric acid
(neutralized) as a sole carbon source.

| | growth score |
|---|---|
| MG1655 | +++ |
| GLUT1-3 | None |
| GLUT1-9 | None |
| GLUT1-10 | None |
| GLUT2-1 | None |
| GLUT2-9 | None |
| GLUT2-10 | None |
| GLUT3-5 | None |
| GLUT3-7 | None |
| GLUT3-9 | None |
| GLUT4-1 | None |
| GLUT4-4 | None |
| GLUT4-10 | None |
| GLUT5-4 | None |
| GLUT5-5 | None |
| GLUT6-4 | None |
| GLUT6-5 | None |
| GLUT6-10 | None |
| GLUT7-2 | None |
| GLUT7-6 | None |
| GLUT7-7 | None |
| GLUT8-5 | None |
| GLUT8-6 | None |
| GLUT8-9 | + |

### Knockout Strain Growth Performance

### High Glutaric Acid Concentrations:

Probable loss-of-function mutations were identified from re-sequencing results as described in methods and in the results of the resequencing analysis. Because there was probable loss-of-function of kgtP in all resequenced isolates, this was the only single knockout tested, and additional knockouts were selected to be tested as double combinations together with kgtP (Table 13). Only the triple knockout in kgtP, proV, and nagC was tested initially due to one isolate (GLUT8-6) possessing probable loss-of-function mutations in kgtP, proV, and nagA, and due to previous studies indicating similar phenotypes and likely the same mechanism of action for improved tolerance in high osmotic pressures (or due to high Na⁺ concentrations) from both nagC and nagA knockouts (Lennen and Herrgard, 2014). All strains with kgtP knockouts exhibited higher growth rates than the wild-type in 23.8 g/L and 47.5 g/L glutaric acid, however none of the multiple knockout strains exhibited significantly improved growth relative to the single kgtP knockout strain alone. K-12 MG1655 ΔkgtP nagC::kan exhibited reduced growth relative to K-12 MG1655 kgtP::kan in 47.5 g/L glutaric acid.

TABLE 13
Growth rates and lag times of preliminary selections of single, double,
and triple gene knockout mutants in M9 + 23.8 g/L or 47.5 g/L glutaric
acid (neutralized), as measured in the Growth Profiler testing format.

| | 23.8 g/L glutarate | | | | 47.5 g/L glutarate | | | |
|---|---|---|---|---|---|---|---|---|
| | mean | | std. error | | mean | | std. error | |
| | μ | t_{lag} | μ | t_{lag} | μ | t_{lag} | μ | t_{lag} |
| strain | (h⁻¹) | (h) | (h⁻¹) | (h) | (h⁻¹) | (h) | (h⁻¹) | (h) |
| MG1655 | 0.466 | 7.4 | 0.010 | 0.1 | 0.101 | 21.8 | 0.032 | 0.7 |
| MG1655 kgtP::kan | 0.487 | 6.1 | 0.019 | 0.0 | 0.191 | 13.5 | 0.019 | 0.4 |
| MG1655 ΔkgtP proV::kan | 0.520 | 6.2 | 0.019 | 0.1 | 0.251 | 13.0 | 0.019 | 0.2 |
| MG1655 ΔkgtP nagC::kan | 0.491 | 6.7 | 0.019 | 0.1 | 0.179 | 15.0 | 0.007 | 0.5 |
| MG1655 ΔkgtP rnb::kan | 0.505 | 6.3 | 0.012 | 0.0 | 0.183 | 13.6 | 0.017 | 0.8 |
| MG1655 ΔkgtP sspA::kan | 0.473 | 6.1 | 0.007 | 0.1 | 0.225 | 12.2 | 0.008 | 0.4 |
| MG1655 ΔkgtP ΔproV nagC::kan | 0.541 | 6.6 | 0.006 | 0.2 | 0.215 | 14.0 | 0.016 | 0.9 |

A second group of selected single, double, and triple knockout mutants was tested in the Biolector testing format in M9+47.5 g/L glutaric acid (Table 14). The ybjL loss-of-function which had been identified from resequencing of adipic acid evolved isolates was included, to determine if that mutation would also confer tolerance toward glutaric acid. The proV and ybjL mutations did not increase growth rates alone, but in double combinations with the kgtP mutation, were found to increase the growth rate over that of the kgtP single mutant. K-12 MG1655 ΔkgtP sspA::kan additionally exhibited an increased growth rate over that of the kgtP single knockout mutant. The triple knockout mutant in kgtP, proV, and ybjL exhibited a growth rate higher than that of the tested double knockout combinations, with a growth rate nearly equivalent to two of the evolved isolates tested alongside in the same experiment (GLUT1-3 and GLUT4-1), and exceeding the growth rate of many other evolved isolates in Table 6.

TABLE 14
Growth rates and lag times of selected single, double, and
triple gene knockout mutants in M9 + 47.5 g/L glutaric
acid (neutralized), as measured in the Biolector testing format.

| | mean | | std. error | |
|---|---|---|---|---|
| | μ | t_{lag} | μ | t_{lag} |
| Strain | (h⁻¹) | (h) | (h⁻¹) | (h) |
| MG1655 | 0.100 | 10.4 | 0.018 | 2.8 |
| GLUT1-3 | 0.278 | 4.6 | 0.014 | 0.6 |
| GLUT1-10 | 0.442 | 10.8 | 0.009 | 0.1 |
| GLUT4-1 | 0.312 | 7.1 | 0.009 | 0.1 |
| GLUT8-6 | 0.402 | 7.8 | 0.001 | 0.2 |
| MG1655 kgtP::kan | 0.181 | 5.8 | 0.005 | 0.6 |
| MG1655 proV::kan | 0.085 | 0.6 | 0.008 | 2.4 |
| MG1655 ybjL::kan | 0.126 | 12.8 | 0.006 | 0.7 |
| MG1655 ΔkgtP proV::kan | 0.244 | 6.5 | 0.000 | 0.2 |
| MG1655 ΔkgtP sspA::kan | 0.256 | 5.7 | 0.007 | 0.9 |
| MG1655 ΔkgtP ybjL::kan | 0.213 | 6.0 | 0.008 | 0.3 |
| MG1655 ΔkgtP ΔproV ybjL::kan | 0.300 | 7.0 | 0.013 | 0.2 |

### High Adipic Acid Concentrations

Probable loss-of-function mutations were identified as previously described. As for glutarate, probable loss-of-function of kgtP was identified in all resequenced isolates, therefore this single knockout was tested with additional double and triple combinations all containing the kgtP knockout. Of the tested strains, K-12 MG1655 kgtP::kan exhibited slightly improved tolerance in 25 g/L adipate, and a much larger improvement in growth in 50 g/L adipate (Table 15). The only tested combinatorial knockout with a higher growth rate than the kgtP single knockout strain was MG1655 ΔkgtP proV::kan. K-12 MG1655 ΔkgtP sspA::kan exhibited greatly reduced tolerance relative to the wild-type, indicating that the sspA mutations isolated in resequenced mutants are likely either gain-of-function mutations or that they only result in weakened activity of the gene product.

TABLE 15
Growth rates and lag times of preliminary selections of single, double,
and triple gene knockout mutants in M9 + 25 g/L or 50 g/L adipic
acid (neutralized), as measured in the Growth Profiler testing format.

| | 25 g/L adipate | | | | 50 g/L adipate | | | |
|---|---|---|---|---|---|---|---|---|
| | mean | | std. error | | mean | | std. error | |
| | μ | t_{lag} | μ | t_{lag} | μ | t_{lag} | μ | t_{lag} |
| strain | (h⁻¹) | (h) | (h⁻¹) | (h) | (h⁻¹) | (h) | (h⁻¹) | (h) |
| MG1655 | 0.467 | 6.9 | 0.015 | 0.1 | 0.115 | 25.7 | 0.004 | 0.7 |
| MG1655 kgtP::kan | 0.534 | 6.4 | 0.003 | 0.2 | 0.192 | 17.5 | 0.002 | 0.6 |
| MG1655 ΔkgtP proV::kan | 0.583 | 6.2 | 0.029 | 0.2 | 0.264 | 16.5 | 0.005 | 0.2 |
| MG1655 ΔkgtP pstS::kan | 0.465 | 7.6 | 0.006 | 0.2 | 0.139 | 21.3 | 0.005 | 0.3 |
| MG1655 ΔkgtP sspA::kan | 0.525 | 6.8 | 0.006 | 0.4 | 0.117 | 49.2 | 0.029 | 12.4 |
| MG1655 ΔkgtP ΔproV nagC::kan | 0.501 | 6.8 | 0.008 | 0.2 | 0.213 | 19.7 | 0.009 | 0.7 |

In a second experiment, additional single, double, and triple knockout combinations were tested in 25 g/L and 50 g/L adipate (Table 16).

TABLE 16
Growth rates and lag times of additional selections of single, double,
and triple gene knockout mutants in M9 + 25 g/L or 50 g/L adipic
acid (neutralized), as measured in the Growth Profiler testing format.

| | 25 g/L adipate | | | | 50 g/L adipate | | | |
|---|---|---|---|---|---|---|---|---|
| | mean | | std. error | | mean | | std. error | |
| | μ | t_{lag} | μ | t_{lag} | μ | t_{lag} | μ | t_{lag} |
| strain | (h⁻¹) | (h) | (h⁻¹) | (h) | (h⁻¹) | (h) | (h⁻¹) | (h) |
| MG1655 | 0.558 | 7.5 | 0.030 | 0.1 | 0.080 | 52.5 | 0.024 | 2.8 |
| MG1655 kgtP::kan | 0.575 | 6.3 | 0.017 | 0.2 | 0.189 | 16.0 | 0.002 | 0.6 |
| MG1655 ΔkgtP proV::kan | 0.608 | 6.2 | 0.032 | 0.2 | 0.255 | 15.1 | 0.010 | 0.7 |
| MG1655 ybjL::kan | 0.516 | 7.4 | 0.020 | 0.1 | 0.142 | 26.1 | 0.010 | 1.0 |
| MG1655 ΔkgtP ybjL::kan | 0.552 | 6.3 | 0.029 | 0.1 | 0.254 | 13.5 | 0.006 | 0.5 |
| MG1655 ΔkgtP ΔproV ybjL::kan | 0.586 | 6.3 | 0.011 | 0.2 | 0.326 | 12.6 | 0.019 | 0.7 |

The ybjL single knockout strain was found to moderately improve tolerance, but below the levels conferred by deletion of kgtP. K-12 MG1655 ΔkgtP ybjL::kan exhibited growth rates similar to K-12 MG1655 ΔkgtP proV::kan, and the triple knockout combination found in K-12 MG1655 ΔkgtP ΔproV ybjL::kan exhibited an improved growth rate over either double knockout combination. Selected single, double, and triple knockout strains were then tested in the Biolector testing format (Table 17). Results were similar to that observed in Table 16, with a higher growth rate observed for K-12 MG1655 ΔkgtP ΔproV ybjL::kan than for some ALE isolates. K-12 MG1655 ΔkgtP sspA::kan was additionally tested, however growth rates were not improved for media containing adipate as they were for glutarate.

TABLE 17
Growth rates and lag times of selected single, double, and
triple gene knockout mutants in M9 + 50 g/L adipic acid
(neutralized), as measured in the Biolector testing format.

| | mean | | std. error | |
|---|---|---|---|---|
| | μ | t_{lag} | μ | t_{lag} |
| strain | (h⁻¹) | (h) | (h⁻¹) | (h) |
| MG1655 | 0.188 | 13.8 | 0.030 | 1.4 |
| ADIP1-9 | 0.284 | 8.1 | 0.011 | 9.0 |
| ADIP4-8 | 0.351 | 6.1 | 0.006 | 0.2 |
| ADIP6-3 | 0.311 | 6.3 | 0.008 | 0.2 |
| ADIP8-3 | 0.208 | 4.5 | 0.004 | 2.7 |
| MG1655 kgtP::kan | 0.225 | 11.5 | 0.004 | 0.4 |
| MG1655 proV::kan | 0.153 | 19.2 | 0.003 | 0.5 |
| MG1655 ybjL::kan | 0.195 | 10.9 | 0.005 | 0.0 |
| MG1655 ΔkgtP proV::kan | 0.262 | 11.0 | 0.007 | 0.1 |
| MG1655 ΔkgtP ybjL::kan | 0.229 | 10.4 | 0.007 | 0.7 |
| MG1655 ΔkgtP sspA::kan | 0.164 | 20.5 | 0.056 | 10.0 |
| MG1655 ΔkgtP ΔproV ybjL::kan | 0.298 | 9.7 | 0.034 | 2.2 |

### Screening of a Single Deletion Mutant Collection for Diacid Tolerance

To determine if any additional single gene deletion candidates were overlooked, screening on elevated glutarate and adipate concentrations was also conducted using the Keio collection of gene knockouts, which is a commercial collection of knockouts in nearly all non-essential genes and ORFs in E. coli strain BW25113. This strain is a K-12 derivative and possesses known mutations relative to the K-12 MG1655 background. All Keio collection strains with knockouts in genes that were found to be mutated in Tables 8 and 9 were screened for growth against the BW25113 control in M9+40 g/L or 47.5 g/L glutaric acid, or M9+45 g/L or 50 g/L adipic acid (neutralized with sodium hydroxide) in the Growth Profiler screening format. Primary screening hits were measured again in a secondary screen in biological replicates, with averaged growth curves for 3 biological replicate cultures shown individually for each strain in Tables 18 and 19, For glutaric acid (Table 18), BW25113 cspE::kan and BW25113 proX::kan exhibited the largest increases in growth rate at 47.5 g/L glutarate, with small improvements also seen with 40 g/L glutarate. ProX is a subunit with ProV in the ProVWX ABC transporter. In M9+47.5 g/L glutarate, additional knockout strains with smaller improvements in growth rates were the rfaE, yfbP, and yfjM knockout strains.

For adipic acid (Table 19), a number of single deletion mutants exhibited moderate increases in growth rate in 50 g/L adipate. These were knockouts in proQ, pstS, rph, rpoS, sspA, tdk, uvrB, ycjG, and yeaR. In 47.5 g/L adipate, smaller percentage improvements in growth rate were observed, however all of these mutants similar exhibited significant increases in growth rate.

TABLE 18
Growth rates of Keio collection knockouts in M9 + 40
g/L and 47.5 g/L glutaric acid (neutralized with sodium
hydroxide) as measured in the Growth Profiler testing format.

| | 40 g/L glutarate | | 47.5 g/L glutarate | |
|---|---|---|---|---|
| | μ | std. | μ | std. |
| Strain | (h⁻¹) | error | (h⁻¹) | error |
| BW25113 | 0.227 | 0.010 | 0.090 | 0.011 |
| BW25113 kgtP::kan | 0.346 | — | 0.278 | — |
| BW25113 cspE::kan | 0.248 | 0.012 | 0.138 | 0.008 |
| BW25113 greA::kan | 0.228 | 0.017 | 0.094 | 0.003 |
| BW25113 mprA::kan | 0.219 | 0.017 | 0.096 | 0.015 |
| BW25113 polB::kan | 0.205 | 0.012 | 0.097 | 0.005 |
| BW25113 proX::kan | 0.261 | 0.005 | 0.140 | 0.011 |
| BW25113 rfaE::kan | 0.220 | 0.006 | 0.121 | 0.007 |
| BW25113 ssuA::kan | 0.207 | 0.007 | 0.093 | 0.005 |
| BW25113 yfbP::kan | 0.216 | 0.009 | 0.118 | 0.009 |
| BW25113 yfjM::kan | 0.228 | 0.006 | 0.118 | 0.004 |
| BW25113 ygdH::kan | 0.215 | 0.014 | 0.098 | 0.007 |
| BW25113 yiaU::kan | 0.235 | 0.005 | 0.111 | 0.009 |

TABLE 19
Growth rates of Keio collection knockouts in M9 +
45 g/L and 50 g/L adipic acid (neutralized with sodium
hydroxide) as measured in the Growth Profiler testing format.

| | |
|---|---|
| 45 g/L adipate | 50 g/L adipate |

| | μ | std. | μ | std. |
|---|---|---|---|---|
| strain | (h⁻¹) | error | (h⁻¹) | error |
| BW25113 | 0.158 | 0.009 | 0.072 | 0.010 |
| BW25113 kgtP::kan | 0.230 | — | 0.218 | — |
| BW25113 idnR::kan | 0.165 | 0.014 | 0.070 | 0.001 |
| BW25113 lsrK::kan | 0.154 | 0.006 | 0.070 | 0.017 |
| BW25113 malQ::kan | 0.162 | 0.017 | 0.077 | 0.002 |
| BW25113 metL::kan | 0.167 | 0.003 | 0.067 | 0.036 |
| BW25113 nrfG::kan | 0.174 | 0.007 | 0.064 | 0.005 |
| BW25113 proQ::kan | 0.189 | 0.016 | 0.096 | 0.006 |
| BW25113 pstS::kan | 0.201 | 0.011 | 0.107 | 0.011 |
| BW25113 rph::kan | 0.206 | 0.005 | 0.119 | 0.012 |
| BW25113 rpoS::kan | 0.189 | 0.002 | 0.113 | 0.001 |
| BW25113 sspA::kan | 0.186 | 0.003 | 0.116 | 0.021 |
| BW25113 tdk::kan | 0.205 | 0.006 | 0.113 | 0.003 |
| BW25113 uvrB::kan | 0.197 | 0.016 | 0.113 | 0.019 |
| BW25113 ycjG::kan | 0.195 | 0.015 | 0.127 | 0.019 |
| BW25113 yeaR::kan | 0.197 | 0.011 | 0.101 | 0.023 |

A list of all gene disruption mutants in both the K-12 MG1655 and BW25113 background strains that exhibited increased tolerance to glutaric acid is shown in Table 20. A similar table for adipic acid is shown in Table 21.

TABLE 20
Summary of knockout strains with improved growth
over the wild-type strain in glutaric acid

| | Growth rate | Lag time |
|---|---|---|
| Strain genotype | improvement | improvement |
| K-12 MG1655 kgtP::kan | Moderate | large |
| K-12 MG1655 ybjL::kan | Small | none |
| K-12 MG1655 ΔkgtP proV::kan | Moderate | large |
| K-12 MG1655 ΔkgtP ybjL::kan | Moderate | large |
| K-12 MG1655 ΔkgtP sspA::kan | Moderate | large |
| K-12 MG1655 ΔkgtP ΔproV ybjL::kan | Large | large |
| BW25113 kgtP::kan | Large (40 and 47.5 g/L) | not quantified |
| BW25113 cspE::kan | small (40 and 47.5 g/L) | not quantified |
| BW25113 proX::kan | small (40 and 47.5 g/L) | not quantified |
| BW25113 rfaE::kan | minor (47.5 g/L) | not quantified |
| BW25113 yfbP::kan | minor (47.5 g/L) | not quantified |
| BW25113 yfjM::kan | minor (47.5 g/L) | not quantified |

TABLE 21
Summary of knockout strains with improved growth
over the wild-type strain in adipic acid

| | Growth rate | Lag time |
|---|---|---|
| Strain genotype | improvement | improvement |
| K-12 MG1655 kgtP::kan | Moderate | large |
| K-12 MG1655 ybjL::kan | Small | none |
| K-12 MG1655 ΔkgtP proV::kan | Moderate | large |
| K-12 MG1655 ΔkgtP ybjL::kan | Moderate | large |
| K-12 MG1655 ΔkgtP ΔproV ybjL::kan | Large | large |
| BW25113 kgtP::kan | Large (45 and 50 g/L) | not quantified |
| BW25113 proQ::kan | small (45 and 50 g/L) | not quantified |
| BW25113 pstS::kan | moderate (45 and 50 g/L) | not quantified |
| BW25113 rph::kan | moderate (45 and 50 g/L) | not quantified |
| BW25113 rpoS::kan | small (45 and 50 g/L) | not quantified |
| BW25113 sspA::kan | small (45 and 50 g/L) | not quantified |
| BW25113 tdk::kan | small (45 and 50 g/L) | not quantified |
| BW25113 uvrB::kan | small (45 and 50 g/L) | not quantified |
| BW25113 ycjG::kan | small (45 g/L), moderate (50 g/L) | not quantified |
| BW25113 yeaR::kan | small (45 and 50 g/L) | not quantified |

### Sole Carbon Source Plate Growth Assay of Knockout Strains

The single knockout strains in kgtP, proV, and ybjL, plus the double and triple combination knockout strains, were struck as previously described on glutarate as a sole carbon source, together with wild-type K-12 MG1655 and a selection of ALE evolved isolates as controls. Robust growth was again observed from K-12 MG1655 after a few weeks incubation, with reduced growth of GLUT8-9 and greatly reduced or no growth in other evolved isolates (Table 22). A larger inoculum was spread on the plates, which likely explains why very weak growth was observed for GLUT8-6 and GLUT1-10. K-12 MG1655 kgtP::kan exhibited no growth, indicating that loss-of-function of kgtP is explicitly responsible for the weak or absent growth of evolved strains on glutarate, and suggesting that KgtP is indeed a direct importer of glutrate. The proV and ybjL single knockout strains did not exhibit reduced growth relative to K-12 MG1655, and double and triple knockout combination strains with kgtP did not exhibit any growth, as would be expected from the loss of kgtP.

TABLE 22
Qualitative growth scores of selected controls (K-12 MG1655 and
glutarate evolved isolates) plus selected single, double, and
triple knockout mutants isolates on M9 agar plates containing
10 g/L glutaric acid (neutralized) as a sole carbon source.

| strain | growth score |
|---|---|
| MG1655 | +++ |
| GLUT1-3 | none |
| GLUT1-10 | + |
| GLUT4-1 | none |
| GLUT8-6 | + |
| GLUT8-9 | + |
| MG1655 kgtP::kan | none |
| MG1655 proV::kan | +++ |
| MG1655 ybjL::kan | +++ |
| MG1655 ΔkgtP proV::kan | none |
| MG1655 ΔkgtP ybjL::kan | none |
| MG1655 ΔkgtP ΔproV ybjL::kan | none |

### Cross-Compound Tolerance Testing

Every secondary screened evolved isolate from the glutaric acid and adipic acid evolutions was grown in the presence of every other compound in the study as indicated in the Methods. The normalized t_{OD1(evolved strain)}/t_{OD1(wild-type)} are shown in Tables 23 and 24 for the glutaric acid and adipic acid evolved isolates, respectively. Lower values are indicative a larger improvement in growth of the evolved isolate (left column) in that chemical condition (top row), whereas higher values are indicative of a lower improvement or decrease in growth compared to the wild-type. Averaged ratios across conditions and strains shown at the right and bottom of the plot allow for overall by-chemical and by-strain trends to be observed. Strain names that are followed by an asterisk (*) were not re-sequenced, and strain names in italics were found to be hypermutator strains.

The majority of glutaric acid-evolved isolates exhibit cross-tolerance to adipic acid (notable exceptions were isolates from the GLUT5 population, GLUT1-3, GLUT1-9, and GLUT2-10). Likewise, the majority of adipic acid evolved isolates exhibit cross-tolerance to glutaric acid (notable exceptions were most isolates in the ADIP1, ADIP2, and ADIP3 populations, plus a couple isolates from other populations (ADIP4-4, ADIP5-5, neither of which were resequenced). Isolates with the highest degree of cross-tolerance were GLUT4-10, GLUT8-6, and GLUT8-9. The GLUT8 population was notable for possessing coding mutations in polB and lacking coding mutations in spoT. Adipic acid evolved isolates exhibited a lower overall degree of cross tolerance, with the best performing isolate being ADIP6-9. This isolate most likely has loss-of-function of kgtP, proV, and ybjL, plus coding mutations in proQ (suggested to be loss-of-function from Keio mutant screen) and spoT. The ADIP6 population specifically exhibited a high level of cross-tolerance toward all other acid salts in the study (hexanoate, octanoate, isobutyrate, glutarate, and p-coumarate). Acid cross-tolerance was also evident from many glutaric acid evolved isolates, however high cross-tolerance toward the diamine HMDA and the diols 2,3-butanediol and 1,2-propanediol was evident in a number of isolates. Cross-tolerance toward HMDA in population GLUT8 can be inferred to be due to a mutation present in GLUT8-6 and GLUT8-9 that is not found in GLUT8-5, which in this case is a coding mutation in sapC that is likely causative for HMDA cross-tolerance.

TABLE 23
Normalized t_{OD1(evolved)}/t_{OD1(wild-type)} values for glutaric acid-evolved isolates
grown in the presence of inhibitory concentrations of 12 different chemicals.

| | Buta- | | | 2,3- | putres- | | | iso- | | octa- | 1,2- | | |
|---|---|---|---|---|---|---|---|---|---|---|---|---|---|
| | nol | glutarate | coumarate | butanediol | cine | HMDA | adipate | butyrate | hexanoate | noate | propanediol | NaCl | average |
| GLUT1-3 | 1.31 | 1.94 | 2.24 | 3.06 | 4.02 | 1.00 | 1.97 | 1.58 | 0.78 | 0.86 | 2.03 | 3.16 | 2.00 |
| GLUT1-9 | 1.31 | 1.74 | 2.24 | 3.06 | 4.02 | 1.00 | 1.75 | 1.39 | 0.92 | 0.88 | 1.97 | 3.16 | 1.96 |
| GLUT1-10 | 1.31 | 0.72 | 1.4S | 0.65 | 1.42 | 1.00 | 0.58 | 2.92 | 0.68 | 0.79 | 0.78 | 1.39 | 1.14 |
| GLUT2-1 | 1.12 | 0.68 | 1.47 | 1.32 | 1.10 | 0.99 | 0.63 | 1.61 | 0.86 | 0.92 | 1.00 | 1.54 | 1.10 |
| GLUT2-9 | 1.22 | 0.68 | 1.27 | 1.24 | 0.96 | 1.00 | 0.63 | 1.47 | 0.84 | 0.91 | 0.94 | 1.08 | 1.02 |
| GLUT2-10 | 1.31 | 1.42 | 2.24 | 3.06 | 4.02 | 1.00 | 1.40 | 1.29 | 0.86 | 0.86 | 1.75 | 3.16 | 1.87 |
| GLUT3-5 | 1.31 | 0.58 | 1.05 | 0.98 | 0.90 | 1.00 | 0.53 | 1.21 | 0.70 | 0.52 | 0.81 | 2.28 | 0.99 |
| GLUT3-7 | 1.31 | 0.58 | 0.97 | 1.05 | 0.98 | 0.96 | 0.53 | 1.34 | 0.81 | 0.59 | 0.84 | 1.28 | 0.94 |
| GLUT3-9 | 1.24 | 0.68 | 1.55 | 0.76 | 1.06 | 1.00 | 0.85 | 1.63 | 0.73 | 0.77 | 0.97 | 2.80 | 1.17 |
| GLUT4-1 | 1.31 | 0.76 | 1.43 | 3.06 | 1.15 | 0.53 | 0.68 | 1.34 | 1.14 | 1.34 | 1.41 | 0.92 | 1.26 |
| GLUT4-4 | 1.31 | 0.76 | 1.63 | 3.06 | 1.15 | 0.52 | 0.73 | 1.32 | 1.11 | 1.51 | 1.44 | 1.00 | 1.29 |
| GLUT4-10 | 1.31 | 0.56 | 0.79 | 0.76 | 0.90 | 0.73 | 0.52 | 1.37 | 0.70 | 0.45 | 0.84 | 1.07 | 0.83 |
| GLUT5-4 | 1.31 | 2.82 | 1.17 | 3.06 | 4.02 | 1.00 | 2.48 | 5.08 | 0.81 | 0.96 | 1.56 | 3.16 | 2.29 |
| GLUT5-5 | 1.31 | 2.48 | 0.87 | 3.06 | 4.02 | 1.00 | 1.88 | 2.82 | 0.73 | 0.64 | 1.28 | 3.16 | 1.94 |
| GLUT5-9 | 1.31 | 2.78 | 1.26 | 3.06 | 4.02 | 1.00 | 2.47 | 5.08 | 0.76 | 0.41 | 1.47 | 3.16 | 2.23 |
| GLUT6-4 | 1.16 | 0.70 | 1.01 | 1.14 | 1.17 | 1.00 | 0.67 | 1.37 | 0.86 | 0.81 | 0.94 | 1.70 | 1.04 |
| GLUT6-5 | 1.27 | 0.74 | 1.05 | 1.38 | 1.33 | 0.90 | 0.67 | 1.39 | 0.95 | 0.83 | 1.06 | 1.82 | 1.12 |
| GLUT6-10 | 1.25 | 0.68 | 0.84 | 1.51 | 1.00 | 1.00 | 0.63 | 1.29 | 0.97 | 0.81 | 1.06 | 0.98 | 1.00 |
| GLUT7-2 | 1.22 | 0.64 | 0.64 | 1.48 | 0.88 | 0.60 | 0.57 | 1.21 | 0.84 | 0.79 | 1.00 | 0.85 | 0.89 |
| GLUT7-6 | 1.20 | 0.62 | 0.77 | 1.03 | 0.96 | 0.39 | 0.65 | 1.29 | 0.92 | 0.70 | 1.09 | 1.00 | 0.88 |
| GLUT7-7 | 1.16 | 0.62 | 1.12 | 0.83 | 1.04 | 0.75 | 0.58 | 1.71 | 0.68 | 0.55 | 0.88 | 1.13 | 0.92 |
| GLUT8-5 | 1.31 | 0.94 | 1.00 | 1.03 | 2.29 | 1.00 | 0.83 | 0.92 | 0.70 | 0.80 | 0.94 | 3.05 | 1.23 |
| GLUT8-6 | 1.31 | 0.60 | 1.00 | 1.21 | 0.90 | 0.33 | 0.53 | 1.18 | 0.70 | 0.48 | 0.84 | 0.90 | 0.83 |
| GLUT8-9 | 1.31 | 0.60 | 0.91 | 0.94 | 0.96 | 0.34 | 0.52 | 1.03 | 0.68 | 0.48 | 0.88 | 0.95 | 0.80 |
| average | 1.27 | 1.06 | 1.25 | 1.74 | 1.84 | 0.83 | 0.97 | 1.79 | 0.82 | 0.78 | 1.16 | 1.86 | 1.28 |
| # > wt | 0 | 18 | 7 | 6 | 8 | 11 | 18 | 1 | 22 | 22 | 11 | 5 | |
| % > wt | 0.0 | 75.0 | 29.2 | 25.0 | 33.3 | 45.8 | 75.0 | 4.2 | 91.7 | 91.7 | 45.8 | 20.8 | |

TABLE 24
Normalized t_{OD1(evolved)}/t_{OD1(wild-type)} values for adipic acid-evolved isolates
grown in the presence of inhibitory concentrations of 12 different chemicals.

| | buta- | | | 2,3- | putres- | | | iso- | | octa- | 1,2- | | |
|---|---|---|---|---|---|---|---|---|---|---|---|---|---|
| | nol | glutarate | courmarate | butanediol | cine | HMDA | Adipate | butyrate | hexanoate | noate | propanediol | NaCl | average |
| ADIP1-1 | 1.22 | 1.38 | 1.75 | 0.59 | 3.22 | 3.51 | 1.37 | 4.20 | 0.74 | 1.38 | 5.68 | 2.64 | 2.31 |
| ADIP1-6* | 1.25 | 0.96 | 1.75 | 0.59 | 3.22 | 3.51 | 0.73 | 3.13 | 1.00 | 1.24 | 0.74 | 2.64 | 1.73 |
| ADIP1-9 | 1.27 | 1.32 | 1.75 | 0.58 | 3.22 | 3.51 | 1.34 | 4.20 | 0.81 | 1.38 | 0.79 | 2.64 | 1.90 |
| ADIP2-5 | 1.17 | 1.09 | 1.06 | 0.87 | 2.77 | 3.51 | 1.28 | 0.87 | 0.67 | 1.38 | 5.68 | 2.64 | 1.92 |
| ADIP2-6 | 1.22 | 0.95 | 1.09 | 0.71 | 2.48 | 3.51 | 0.96 | 0.87 | 0.63 | 1.38 | 0.79 | 2.64 | 1.44 |
| ADIP2-10 | 1.18 | 1.32 | 0.99 | 1.10 | 2.87 | 3.51 | 1.16 | 0.91 | 0.65 | 1.38 | 3.26 | 2.64 | 1.75 |
| ADIP3-2 | 1.27 | 0.90 | 1.42 | 0.51 | 3.22 | 3.51 | 0.94 | 3.67 | 0.74 | 1.38 | 0.68 | 2.64 | 1.74 |
| ADIP3-4 | 1.27 | 1.35 | 1.75 | 0.59 | 3.22 | 3.51 | 1.16 | 4.20 | 0.84 | 1.38 | 1.24 | 2.64 | 1.93 |
| ADIP3-8 | 1.18 | 0.85 | 1.35 | 0.50 | 3.22 | 3.51 | 1.37 | 3.91 | 0.74 | 1.38 | 0.85 | 2.64 | 1.79 |
| ADIP4-1 | 1.27 | 0.50 | 1.15 | 0.73 | 0.93 | 1.00 | 0.47 | 1.30 | 0.84 | 0.94 | 1.15 | 0.95 | 0.94 |
| ADIP4-4* | 1.27 | 0.96 | 0.83 | 1.01 | 1.02 | 1.07 | 0.71 | 1.28 | 1.05 | 1.03 | 1.79 | 2.64 | 1.22 |
| ADIP4-8 | 1.26 | 0.53 | 0.76 | 0.83 | 1.00 | 1.11 | 1.80 | 1.33 | 0.88 | 0.78 | 1.03 | 0.86 | 1.01 |
| ADIP5-2 | 1.12 | 0.41 | 0.96 | 1.87 | 0.75 | 0.73 | 0.32 | 1.13 | 0.91 | 0.91 | 5.29 | 2.64 | 1.42 |
| ADIP5-5* | 1.27 | 1.06 | 0.98 | 1.87 | 1.02 | 1.11 | 0.93 | 1.15 | 1.26 | 0.89 | 1.18 | 1.22 | 1.16 |
| ADIP5-6 | 1.18 | 1.37 | 0.77 | 0.77 | 3.22 | 3.51 | 1.17 | 2.07 | 0.88 | 0.81 | 1.97 | 2.64 | 1.70 |
| ADIP6-3 | 1.16 | 0.42 | 0.55 | 1.87 | 0.90 | 1.27 | 0.40 | 1.91 | 0.70 | 0.53 | 1.12 | 0.79 | 0.97 |
| ADIP6-9 | 1.09 | 0.45 | 0.55 | 0.62 | 0.88 | 1.11 | 0.38 | 1.30 | 0.70 | 0.56 | 0.91 | 0.84 | 0.78 |
| ADIP6-10 | 1.12 | 0.58 | 0.89 | 0.68 | 1.02 | 1.11 | 0.47 | 3.35 | 0.84 | 0.89 | 1.47 | 1.42 | 1.15 |
| ADIP7-2 | 1.27 | 0.53 | 0.63 | 0.73 | 1.22 | 1.55 | 0.47 | 0.91 | 0.84 | 1.38 | 1.00 | 1.07 | 0.96 |
| ADIP7-5 | 1.27 | 0.68 | 1.13 | 0.59 | 1.92 | 1.93 | 0.56 | 3.93 | 0.77 | 1.06 | 1.29 | 2.47 | 1.47 |
| ADIP7-10* | 1.27 | 0.54 | 0.89 | 0.46 | 2.12 | 2.13 | 0.53 | 3.50 | 0.72 | 0.99 | 0.71 | 1.81 | 1.30 |
| ADIP8-3 | 1.27 | 0.63 | 0.55 | 1.10 | 1.35 | 1.96 | 0.56 | 1.41 | 1.00 | 1.38 | 1.38 | 2.64 | 1.27 |
| ADIP8-7 | 1.27 | 0.63 | 0.46 | 0.73 | 1.02 | 1.40 | 0.47 | 1.52 | 0.88 | 1.29 | 1.97 | 2.64 | 1.19 |
| ADIP8-10 | 1.27 | 0.60 | 0.48 | 0.73 | 1.42 | 1.55 | 0.45 | 1.09 | 0.81 | 1.38 | 1.06 | 1.03 | 0.99 |
| average | 1.22 | 0.83 | 1.02 | 0.86 | 1.97 | 2.25 | 0.83 | 2.21 | 0.83 | 1.13 | 1.79 | 2.06 | 1.42 |
| # > wt | 0 | 17 | 14 | 18 | 4 | 1 | 16 | 4 | 20 | 9 | 7 | 4 | |
| % > wt | 0.0 | 70.8 | 58.3 | 75.0 | 16.7 | 4.2 | 66.7 | 16.7 | 83.3 | 37.5 | 29.2 | 16.7 | |

Additionally, each evolved isolate was tested for cross-tolerance toward other dicarboxylic acids of interest. First, K-12 MG1655 was tested in the Growth Profiler screening format for growth in the presence of a range of concentrations of each compound (note that this had been done in the Biolector format previously for pimelic acid and sebacic acid (Table 7) thus was not repeated here): fumaric acid, itaconic acid, malic acid, and succinic acid. All diacids tested were either the neutral sodium salts, or the free diacid was neutralized with sodium hydroxide to pH 7.0 for testing. Variable concentrations of these compounds elicited growth inhibition in E. coli K-12 MG1655 (Table 25). Based on these results, a screening concentration was selected for the evolved isolates for which wild-type cells could achieve at a growth rate of 0.15-0.3 h⁻¹ (versus uninhibited growth at 0.7-0.9 h⁻¹ in M9 glucose minimal medium). These concentrations were: 45 g/L fumaric acid, 45 g/L itaconic acid, 55 g/L malic acid, 50 g/L succinic acid, 45 g/L pimelic acid, and 38 g/L sebacic acid. The results of glutaric acid and adipic acid-evolved isolates grown in these concentrations of fumaric, itaconic, and malic acid are shown in are shown in Tables 26 and 27, and the same isolates grown at the selected concentration of succinic, pimelic, and sebacic acid (linear aliphatic diacids) are shown in Tables 28 and 29. A majority of evolved isolates exhibited increased growth rates and/or reductions in lag time in all tested diacids. These in particular included isolates from the ADIP1, ADIP2, ADIP3, and ADIP7 populations, ADIP5-6 (a hypermutator strain) for all diacids generally. ADIP7-2 and ADIP7-5 exhibited the highest growth rates in sebacic acid, with the ADIP2 population also exhibiting significantly improved growth. ADIP7-2 and ADIP7-5 possessed different sets of mutations but notably only possessed loss-of-function mutations in kgtP plus distinct modulatory mutations (probable reduction-of-function of rpoS based on the Keio collection screen and loss-of-function of pstS plus an intergenic insertion between hns and tdk in ADIP7-2, and loss-of-function of ybjL, proV, and probable reduction-of-function of sspA based on the Keio collection screen in ADIP7.5). The common features of ADIP2 isolates were possessing only loss-of-function mutations in kgtP, deletion of proVWX, and mutations that restore expression of PyrE. Glutaric acid evolved isolates tended to exhibit a specificity toward tolerance to particular diacids. Isolates from the GLUT5 population exhibited significantly improved growth rates in fumaric acid (these isolates exhibited probable reduction-of-function of sspA and the RpoB-K203T and SpoT-R236L mutations), whereas the GLUT1 and GLUT2 populations had some of the most improved growth rates and reduced lag times in itaconic acid (these isolates featured loss-of-function of kgtP and the SpoT-V422A and RpoC-H419P mutations in GLUT1-3 and GLUT1-9; loss-of-function of kgtP, proV, probable reduction-of-function of sspA, and the SpoT-V422A mutation in GLUT1-10; loss-of-function of kgtP and optionally proV, and the SpoT-A451D mutation in GLUT2-1 and GLUT2-9; and loss-of-function of kgtP and proV plus the SpoT-A451D and RpoC-H419P mutations in GLUT2-10). Malic acid cross-tolerance was weak across all glutaric acid evolved isolates. Isolates from the GLUT8 population had dramatically improved growth rates (as well as moderate reductions in lag time) toward sebacic acid (these isolates commonly featured loss-of-function of kgtP and proV, and the RpoC-P64L and PolB-R477G mutations, with the best performing isolates GLUT8-6 and GLUT8-9 additionally possessing the SapC-G79W mutation), with GLUT1-10 and GLUT2-9 also exhibiting significantly enhanced growth rates. Notably, the GLUT8 population had relatively poor cross-tolerance for most diacids, and all tested isolates featured the PolB-R477G mutation, whereas all other populations featured isolates with mutations in SpoT.

TABLE 25
Growth rates and lag times of K-12 MG1655 in varying concentrations of the sodium salts of fumaric,
itaconic, malic, and succinic acid, as measured in the Growth Profiler testing format.

| | fumaric acid | | | | itaconic acid | | | | malic acid | | | | succinic acid | | | |
|---|---|---|---|---|---|---|---|---|---|---|---|---|---|---|---|---|
| | mean | | std. error | | mean | | std. error | | mean | | std. error | | mean | | std. error | |
| diacid | μ | t_{lag} | μ | t_{lag} | μ | t_{lag} | μ | t_{lag} | μ | t_{lag} | μ | t_{lag} | μ | t_{lag} | μ | t_{lag} |
| (g/L) | (h¹) | (h) | (h¹) | (h) | (h¹) | (h) | (h¹) | (h) | (h¹) | (h) | (h¹) | (h) | (h¹) | (h) | (h¹) | (h) |
| 0 | 0.747 | 5.1 | 0.030 | 0.2 | 0.747 | 5.1 | 0.030 | 0.2 | 0.747 | 5.1 | 0.030 | 0.2 | 0.747 | 5.1 | 0.030 | 0.2 |
| 10 | 0.662 | 5.8 | 0.021 | 0.0 | 0.703 | 5.8 | 0.037 | 0.1 | 0.686 | 5.9 | 0.053 | 0.0 | 0.665 | 5.4 | 0.084 | 0.0 |
| 20 | 0.615 | 6.9 | 0.019 | 0.0 | 0.603 | 7.0 | 0.036 | 0.2 | 0.639 | 6.6 | 0.024 | 0.1 | 0.610 | 6.7 | 0.024 | 0.1 |
| 30 | 0.482 | 9.5 | 0.009 | 0.1 | 0.520 | 9.4 | 0.012 | 0.2 | 0.526 | 8.0 | 0.002 | 0.2 | 0.496 | 8.9 | 0.028 | 0.1 |
| 40 | 0.285 | 15.4 | 0.005 | 0.2 | 0.320 | 14.1 | 0.014 | 0.2 | 0.410 | 11.1 | 0.005 | 0.2 | 0.345 | 13.4 | 0.014 | 0.1 |
| 50 | 0.116 | 30.2 | 0.011 | 1.0 | 0.117 | 29.9 | 0.008 | 0.7 | 0.287 | 16.9 | 0.002 | 0.2 | 0.179 | 22.4 | 0.008 | 0.0 |
| 60 | — | — | — | — | — | — | — | — | 0.154 | 28.3 | 0.006 | 0.3 | 0.127 | 42.5 | 0.016 | 1.6 |

TABLE 26
Growth rates and lag times of K-12 MG1655 and glutaric acid-evolved isolates in specified
inhibitory concentrations of fumaric, itaconic, and malic acid (sodium salts or neutralized
with sodium hydroxide to pH 7.0), as measured in the Growth Profiler testing format.

| | 45 g/L fumaric acid | | | | 45 g/L itaconic acid | | | | 55 g/L malic acid | | | |
|---|---|---|---|---|---|---|---|---|---|---|---|---|
| | mean | | std. error | | mean | | std. error | | mean | | std. error | |
| | μ | t_{lag} | μ | t_{lag} | μ | t_{lag} | μ | t_{lag} | μ | t_{lag} | μ | t_{lag} |
| strain | (h⁻¹) | (h) | (h⁻¹) | (h) | (h⁻¹) | (h) | (h⁻¹) | (h) | (h⁻¹) | (h) | (h⁻¹) | (h) |
| MG1655 | 0.216 | 16.6 | 0.017 | 0.3 | 0.248 | 13.4 | 0.014 | 0.4 | 0.235 | 17.1 | 0.008 | 0.3 |
| GLUT1-3 | 0.293 | 13.3 | 0.019 | 1.5 | 0.454 | 10.2 | 0.039 | 1.2 | 0.294 | 13.5 | 0.044 | 1.5 |
| GLUT1-9 | 0.274 | 14.9 | 0.028 | 1.2 | 0.446 | 10.4 | 0.028 | 0.4 | 0.259 | 15.3 | 0.019 | 0.9 |
| GLUT1-10 | 0.289 | 18.9 | 0.015 | 7.2 | 0.446 | 18.1 | 0.028 | 13.7 | 0.286 | 19.2 | 0.027 | 8.0 |
| GLUT2-1 | 0.277 | 13.5 | 0.023 | 0.7 | 0.385 | 10.8 | 0.036 | 0.3 | 0.273 | 13.7 | 0.025 | 0.4 |
| GLUT2-9 | 0.267 | 12.2 | 0.007 | 0.4 | 0.415 | 9.0 | 0.028 | 0.3 | 0.270 | 12.5 | 0.021 | 0.4 |
| GLUT2-10 | 0.312 | 12.3 | 0.013 | 1.0 | 0.494 | 8.8 | 0.030 | 0.4 | 0.282 | 12.2 | 0.038 | 0.9 |
| GLUT3-5 | 0.263 | 16.6 | 0.014 | 3.3 | 0.310 | 12.0 | 0.058 | 2.0 | 0.257 | 15.4 | 0.014 | 2.3 |
| GLUT3-7 | 0.264 | 16.2 | 0.023 | 3.3 | 0.326 | 11.7 | 0.017 | 1.7 | 0.234 | 15.5 | 0.053 | 3.5 |
| GLUT3-9 | 0.305 | 24.3 | 0.012 | 0.7 | 0.257 | 14.5 | 0.094 | 1.9 | 0.252 | 20.5 | 0.015 | 1.5 |
| GLUT4-1 | 0.295 | 14.6 | 0.026 | 0.7 | 0.414 | 11.7 | 0.016 | 0.9 | 0.264 | 15.9 | 0.014 | 0.7 |
| GLUT4-4 | 0.287 | 15.3 | 0.015 | 0.4 | 0.339 | 13.4 | 0.110 | 1.6 | 0.264 | 16.5 | 0.012 | 0.2 |
| GLUT4-10 | 0.274 | 16.3 | 0.014 | 1.6 | 0.328 | 13.5 | 0.016 | 3.2 | 0.250 | 15.6 | 0.017 | 1.5 |
| GLUT5-4 | 0.386 | 14.1 | 0.016 | 1.5 | 0.398 | 15.2 | 0.056 | 6.4 | 0.272 | 15.3 | 0.022 | 1.7 |
| GLUT5-5 | 0.375 | 15.1 | 0.008 | 0.2 | 0.368 | 11.4 | 0.078 | 0.8 | 0.275 | 16.6 | 0.032 | 0.5 |
| GLUT5-9 | 0.370 | 14.9 | 0.013 | 0.6 | 0.438 | 22.5 | 0.026 | 2.8 | 0.246 | 16.4 | 0.026 | 0.9 |
| GLUT6-4 | 0.281 | 13.8 | 0.020 | 0.6 | 0.360 | 11.3 | 0.080 | 1.0 | 0.251 | 14.5 | 0.022 | 0.7 |
| GLUT6-5 | 0.279 | 13.2 | 0.007 | 0.4 | 0.420 | 10.6 | 0.007 | 0.5 | 0.241 | 13.8 | 0.014 | 0.8 |
| GLUT6-10 | 0.277 | 14.2 | 0.018 | 0.8 | 0.502 | 10.5 | 0.150 | 0.7 | 0.245 | 14.8 | 0.071 | 1.8 |
| GLUT7-2 | 0.304 | 14.6 | 0.008 | 0.6 | 0.441 | 10.6 | 0.025 | 0.2 | 0.275 | 16.1 | 0.035 | 1.4 |
| GLUT7-6 | 0.261 | 19.5 | 0.007 | 1.1 | 0.293 | 13.7 | 0.015 | 0.3 | 0.247 | 21.9 | 0.015 | 5.5 |
| GLUT7-7 | 0.193 | 22.0 | 0.076 | 2.9 | 0.239 | 8.5 | 0.207 | 7.4 | 0.185 | 19.1 | 0.022 | 1.0 |
| GLUT8-5 | 0.247 | 14.4 | 0.024 | 0.6 | 0.372 | 17.3 | 0.024 | 8.5 | 0.180 | 15.8 | 0.034 | 0.9 |
| GLUT8-6 | 0.272 | 43.5 | 0.107 | 13.5 | 0.236 | 10.3 | 0.206 | 9.2 | 0.208 | 32.7 | 0.047 | 3.7 |
| GLUT8-9 | 0.196 | 54.3 | 0.033 | 9.0 | 0.224 | 12.0 | 0.195 | 10.4 | 0.192 | 47.7 | 0.052 | 15.3 |

TABLE 27
Growth rates and lag times of K-12 MG1655 and adipic acid-evolved
isolates in specified inhibitory concentrations of fumaric, itaconic,
and malic acid (sodium salts or neutralized with sodium hydroxide
to pH 7.0), as measured in the Growth Profiler testing format.

| | 45 g/L fumaric acid | | | | 45 g/L itaconic acid | | | | 55 g/L malic acid | | | |
|---|---|---|---|---|---|---|---|---|---|---|---|---|
| | mean | | std. error | | mean | | std. error | | mean | | std. error | |
| | μ | t_{lag} | μ | t_{lag} | μ | t_{lag} | μ | t_{lag} | μ | t_{lag} | μ | t_{lag} |
| strain | (h⁻¹) | (h) | (h⁻¹) | (h) | (h⁻¹) | (h) | (h⁻¹) | (h) | (h⁻¹) | (h) | (h⁻¹) | (h) |
| MG1655 | 0.212 | 15.5 | 0.012 | 0.8 | 0.257 | 13.0 | 0.016 | 0.4 | 0.222 | 16.5 | 0.031 | 1.6 |
| ADIP1-1 | 0.347 | 10.2 | 0.017 | 0.0 | 0.466 | 8.3 | 0.019 | 0.2 | 0.333 | 12.8 | 0.028 | 1.6 |
| ADIP1-9 | 0.328 | 9.8 | 0.001 | 0.5 | 0.434 | 8.7 | 0.007 | 0.2 | 0.336 | 10.7 | 0.015 | 1.0 |
| ADIP2-5 | 0.358 | 11.4 | 0.040 | 0.3 | 0.440 | 8.3 | 0.028 | 0.3 | 0.338 | 17.9 | 0.027 | 1.1 |
| ADIP2-6 | 0.380 | 11.0 | 0.006 | 0.3 | 0.470 | 8.2 | 0.009 | 0.1 | 0.348 | 16.8 | 0.007 | 1.1 |
| ADIP2-10 | 0.353 | 11.0 | 0.014 | 0.6 | 0.438 | 8.3 | 0.015 | 0.4 | 0.347 | 16.8 | 0.017 | 1.8 |
| ADIP3-2 | 0.327 | 9.4 | 0.011 | 1.1 | 0.459 | 7.9 | 0.004 | 0.9 | 0.318 | 10.7 | 0.016 | 2.1 |
| ADIP3-4 | 0.324 | 9.7 | 0.002 | 0.6 | 0.451 | 8.4 | 0.018 | 0.5 | 0.347 | 9.9 | 0.009 | 0.9 |
| ADIP3-8 | 0.343 | 10.1 | 0.008 | 1.3 | 0.433 | 8.5 | 0.023 | 1.1 | 0.329 | 11.4 | 0.007 | 2.0 |
| ADIP4-1 | 0.264 | 11.9 | 0.005 | 0.4 | 0.339 | 11.2 | 0.023 | 0.6 | 0.275 | 12.1 | 0.006 | 0.6 |
| ADIP4-8 | 0.254 | 12.4 | 0.009 | 1.2 | 0.326 | 10.4 | 0.029 | 1.1 | 0.238 | 13.4 | 0.016 | 2.1 |
| ADIP5-2 | 0.280 | 10.6 | 0.074 | 3.5 | 0.354 | 9.4 | 0.108 | 2.7 | 0.299 | 10.1 | 0.052 | 2.1 |
| ADIP5-6 | 0.444 | 9.0 | 0.017 | 1.0 | 0.527 | 7.2 | 0.020 | 0.6 | 0.404 | 10.2 | 0.018 | 2.4 |
| ADIP6-3 | 0.275 | 11.3 | 0.066 | 1.2 | 0.327 | 9.9 | 0.088 | 0.9 | 0.295 | 11.6 | 0.057 | 1.6 |
| ADIP6-9 | 0.250 | 12.3 | 0.047 | 2.2 | 0.337 | 10.3 | 0.057 | 1.7 | 0.250 | 12.7 | 0.073 | 2.7 |
| ADIP6-10 | 0.308 | 10.0 | 0.026 | 0.2 | 0.343 | 9.1 | 0.059 | 0.6 | 0.323 | 10.0 | 0.026 | 0.6 |
| ADIP7-2 | 0.312 | 10.1 | 0.011 | 0.2 | 0.342 | 7.9 | 0.122 | 1.2 | 0.329 | 10.3 | 0.006 | 0.3 |
| ADIP7-5 | 0.326 | 10.0 | 0.010 | 0.7 | 0.402 | 8.6 | 0.012 | 0.4 | 0.326 | 10.2 | 0.016 | 0.6 |
| ADIP8-3 | 0.202 | 14.4 | 0.003 | 0.3 | 0.308 | 11.7 | 0.006 | 0.1 | 0.194 | 14.4 | 0.038 | 0.5 |
| ADIP8-7 | 0.188 | 16.1 | 0.040 | 2.4 | 0.235 | 12.5 | 0.138 | 1.2 | 0.202 | 14.9 | 0.003 | 0.3 |
| ADIP8-10 | 0.221 | 14.3 | 0.015 | 0.5 | 0.290 | 11.3 | 0.025 | 0.3 | 0.202 | 14.2 | 0.006 | 0.4 |

TABLE 28
Growth rates and lag times of K-12 MG1655 and glutaric acid-evolved isolates in specified
inhibitory concentrations of succinic, pimelic, and sebacic acid (sodium salts or neutralized
with sodium hydroxide to pH 7.0), as measured in the Growth Profiler testing format.

| | 50 g/L succinic acid | | | | 45 g/L pimelic acid | | | | 38 g/L sebacic acid | | | |
|---|---|---|---|---|---|---|---|---|---|---|---|---|
| | mean | | std. error | | mean | | std. error | | mean | | std. error | |
| | μ | t_{lag} | μ | t_{lag} | μ | t_{lag} | μ | t_{lag} | μ | t_{lag} | μ | t_{lag} |
| strain | (h⁻¹) | (h) | (h⁻¹) | (h) | (h⁻¹) | (h) | (h⁻¹) | (h) | (h⁻¹) | (h) | (h⁻¹) | (h) |
| MG1655 | 0.169 | 20.2 | 0.006 | 0.7 | 0.275 | 13.4 | 0.019 | 0.1 | 0.167 | 26.9 | 0.008 | 1.2 |
| GLUT1-3 | 0.231 | 14.5 | 0.025 | 1.8 | 0.369 | 10.4 | 0.028 | 0.8 | 0.074 | 34.9 | 0.006 | 5.4 |
| GLUT1-9 | 0.194 | 20.8 | 0.013 | 8.0 | 0.337 | 11.9 | 0.022 | 0.2 | 0.043 | 44.7 | 0.024 | 8.4 |
| GLUT1-10 | 0.246 | 20.0 | 0.042 | 8.2 | 0.399 | 13.1 | 0.026 | 3.3 | 0.278 | 20.6 | 0.015 | 2.8 |
| GLUT2-1 | 0.232 | 14.7 | 0.016 | 0.2 | 0.362 | 11.6 | 0.002 | 0.4 | 0.153 | 21.7 | 0.005 | 2.7 |
| GLUT2-9 | 0.213 | 14.4 | 0.012 | 0.3 | 0.349 | 10.3 | 0.025 | 0.3 | 0.282 | 12.5 | 0.006 | 1.0 |
| GLUT2-10 | 0.277 | 13.4 | 0.024 | 1.7 | 0.383 | 10.2 | 0.033 | 0.5 | 0.093 | 29.8 | 0.006 | 1.3 |
| GLUT3-5 | 0.242 | 16.1 | 0.020 | 2.1 | 0.280 | 17.6 | 0.096 | 11.1 | 0.106 | 20.0 | 0.132 | 22.4 |
| GLUT3-7 | 0.235 | 15.3 | 0.023 | 2.5 | 0.360 | 11.5 | 0.035 | 1.1 | 0.068 | 6.4 | 0.117 | 11.0 |
| GLUT3-9 | 0.221 | 20.6 | 0.011 | 0.7 | 0.351 | 15.7 | 0.023 | 2.0 | 0.158 | 18.9 | 0.062 | 4.3 |
| GLUT4-1 | 0.263 | 16.3 | 0.016 | 0.9 | 0.378 | 12.8 | 0.035 | 0.4 | 0.266 | 32.9 | 0.005 | 3.6 |
| GLUT4-4 | 0.242 | 16.6 | 0.008 | 0.8 | 0.388 | 13.2 | 0.021 | 0.1 | 0.265 | 34.2 | 0.007 | 0.7 |
| GLUT4-10 | 0.231 | 15.7 | 0.004 | 1.3 | 0.371 | 11.5 | 0.030 | 0.7 | 0.113 | 20.1 | 0.121 | 20.5 |
| GLUT5-4 | 0.281 | 17.0 | 0.038 | 4.8 | 0.433 | 12.1 | 0.026 | 0.4 | 0.183 | 37.7 | 0.014 | 1.1 |
| GLUT5-5 | 0.264 | 18.4 | 0.020 | 0.5 | 0.420 | 12.8 | 0.002 | 0.4 | 0.117 | 54.9 | 0.079 | 4.3 |
| GLUT5-9 | 0.244 | 18.4 | 0.016 | 1.9 | 0.428 | 13.0 | 0.028 | 1.2 | 0.143 | 58.2 | 0.130 | 10.3 |
| GLUT6-4 | 0.241 | 14.2 | 0.031 | 0.5 | 0.356 | 11.7 | 0.022 | 0.6 | 0.085 | 26.4 | 0.012 | 6.6 |
| GLUT6-5 | 0.243 | 13.9 | 0.010 | 0.2 | 0.348 | 11.3 | 0.003 | 0.4 | 0.123 | 21.6 | 0.030 | 3.8 |
| GLUT6-10 | 0.211 | 14.4 | 0.018 | 1.2 | 0.353 | 12.1 | 0.021 | 0.1 | 0.132 | 26.2 | 0.008 | 2.4 |
| GLUT7-2 | 0.223 | 14.9 | 0.031 | 0.4 | 0.349 | 11.6 | 0.030 | 0.3 | 0.173 | 17.5 | 0.010 | 0.9 |
| GLUT7-6 | 0.195 | 17.8 | 0.019 | 1.0 | 0.357 | 14.8 | 0.008 | 0.5 | 0.141 | 18.9 | 0.017 | 1.2 |
| GLUT7-7 | 0.156 | 19.9 | 0.015 | 0.3 | 0.335 | 14.1 | 0.024 | 0.2 | 0.112 | 20.0 | 0.033 | 1.5 |
| GLUT8-5 | 0.198 | 16.4 | 0.042 | 1.2 | 0.349 | 11.8 | 0.012 | 0.4 | 0.300 | 21.4 | 0.026 | 0.9 |
| GLUT8-6 | 0.236 | 34.1 | 0.016 | 7.3 | 0.184 | 20.9 | 0.193 | 18.8 | 0.464 | 13.7 | 0.047 | 0.5 |
| GLUT8-9 | 0.250 | 38.0 | 0.011 | 5.5 | 0.259 | 11.2 | 0.224 | 10.1 | 0.471 | 14.5 | 0.048 | 1.3 |

TABLE 29
Growth rates and lag times of K-12 MG1655 and adipic acid-evolved
isolates in specified inhibitory concentrations of succinic, pimelic,
and sebacic acid (sodium salts or neutralized with sodium hydroxide
to pH 7.0), as measured in the Growth Profiler testing format.

| | 50 g/L succinic acid | | | | 45 g/L pimelic acid | | | | 38 g/L sebacic acid | | | |
|---|---|---|---|---|---|---|---|---|---|---|---|---|
| | mean | | std. error | | mean | | std. error | | mean | | std. error | |
| | μ | t_{lag} | μ | t_{lag} | μ | t_{lag} | μ | t_{lag} | μ | t_{lag} | μ | t_{lag} |
| strain | (h⁻¹) | (h) | (h⁻¹) | (h) | (h⁻¹) | (h) | (h⁻¹) | (h) | (h⁻¹) | (h) | (h⁻¹) | (h) |
| MG1655 | 0.143 | 23.8 | 0.046 | 5.4 | 0.271 | 12.4 | 0.041 | 1.2 | 0.135 | 28.2 | 0.022 | 5.7 |
| ADIP1-1 | 0.299 | 16.1 | 0.012 | 1.5 | 0.432 | 9.6 | 0.040 | 0.3 | 0.244 | 21.6 | 0.029 | 3.8 |
| ADIP1-9 | 0.290 | 14.5 | 0.006 | 2.3 | 0.421 | 8.8 | 0.013 | 0.9 | 0.248 | 17.8 | 0.005 | 1.1 |
| ADIP2-5 | 0.260 | 25.5 | 0.028 | 2.3 | 0.474 | 11.0 | 0.011 | 0.3 | 0.315 | 13.5 | 0.054 | 0.5 |
| ADIP2-6 | 0.278 | 21.7 | 0.013 | 1.1 | 0.480 | 10.6 | 0.006 | 0.2 | 0.319 | 12.9 | 0.019 | 0.3 |
| ADIP2-10 | 0.262 | 22.0 | 0.040 | 4.4 | 0.479 | 10.8 | 0.003 | 1.0 | 0.290 | 13.4 | 0.022 | 0.8 |
| ADIP3-2 | 0.311 | 13.2 | 0.013 | 3.2 | 0.438 | 8.4 | 0.003 | 1.3 | 0.254 | 17.6 | 0.010 | 4.6 |
| ADIP3-4 | 0.308 | 12.7 | 0.006 | 1.6 | 0.435 | 8.5 | 0.010 | 0.8 | 0.262 | 17.1 | 0.007 | 1.3 |
| ADIP3-8 | 0.304 | 13.8 | 0.016 | 2.0 | 0.426 | 9.1 | 0.021 | 1.4 | 0.253 | 19.8 | 0.018 | 4.1 |
| ADIP4-1 | 0.206 | 15.7 | 0.013 | 0.2 | 0.348 | 9.9 | 0.010 | 0.2 | 0.308 | 13.1 | 0.013 | 0.5 |
| ADIP4-8 | 0.228 | 15.8 | 0.037 | 4.0 | 0.349 | 10.3 | 0.011 | 0.8 | 0.289 | 12.8 | 0.008 | 1.3 |
| ADIP5-2 | 0.192 | 13.5 | 0.028 | 2.2 | 0.385 | 8.7 | 0.114 | 2.1 | 0.288 | 15.3 | 0.107 | 4.2 |
| ADIP5-6 | 0.361 | 12.1 | 0.019 | 1.9 | 0.483 | 8.5 | 0.023 | 1.9 | 0.273 | 20.7 | 0.016 | 0.6 |
| ADIP6-3 | 0.246 | 14.9 | 0.065 | 3.7 | 0.346 | 10.4 | 0.106 | 1.2 | 0.268 | 17.9 | 0.125 | 9.4 |
| ADIP6-9 | 0.210 | 18.4 | 0.062 | 5.2 | 0.278 | 11.1 | 0.074 | 1.9 | 0.181 | 23.1 | 0.051 | 7.6 |
| ADIP6-10 | 0.285 | 12.4 | 0.033 | 0.6 | 0.360 | 9.7 | 0.014 | 0.6 | 0.267 | 15.3 | 0.066 | 3.1 |
| ADIP7-2 | 0.294 | 12.5 | 0.018 | 0.3 | 0.393 | 9.0 | 0.012 | 0.4 | 0.356 | 11.1 | 0.014 | 1.1 |
| ADIP7-5 | 0.274 | 12.4 | 0.024 | 1.3 | 0.376 | 9.4 | 0.003 | 0.7 | 0.353 | 12.0 | 0.010 | 1.4 |
| ADIP8-3 | 0.146 | 22.1 | 0.016 | 1.6 | 0.235 | 12.8 | 0.010 | 0.2 | 0.171 | 28.9 | 0.017 | 1.4 |
| ADIP8-7 | 0.163 | 23.2 | 0.014 | 0.7 | 0.268 | 12.8 | 0.060 | 0.6 | 0.157 | 30.9 | 0.012 | 2.2 |
| ADIP8-10 | 0.131 | 20.0 | 0.042 | 0.9 | 0.249 | 12.8 | 0.003 | 0.3 | 0.165 | 30.0 | 0.012 | 2.3 |

### Biological Production of Diacids

Glutaric acid has been the target of two studies, both in engineered E. coli. The highest reported titer of 0.82 g/L from glucose was achieved via native L-lysine production (Adkins et al., 2013) (see FIG. 1 of Adkins et al., 2013, hereby incorporated by reference). A heterologous pathway composed of genes from Pseudomonas putida KT2440 was expressed from plasmids and consisted of a lysine monooxygenase to convert lysine to 5-aminovaleramide, a 5-aminovaleramidase to convert 5-aminovaleramide to 5-aminovaleric acid, a 5-aminovalerate transaminase to convert 5-aminovaleric acid and α-ketoglutarate to glutarate semialdehyde and L-glutamate, and a glutarate semialdehyde dehydrogenase to convert glutarate semialdehyde to glutaric acid. To improve flux toward L-lysine, previously known feedback resistance mutations were made in DapA (4-hydroxytetrahydrodipicolinate synthase) and LysC (asparate kinase III), and these modified proteins were additionally overexpressed from plasmids. Finally, cadA and IdcC, encoding two lysine decarboxylases, were deleted to prevent side conversion of L-lysine into cadaverine. In a second study (Park et al., 2013), glutarate was not able to be produced Prom glucose, however 1.7 g/L glutarate could be produced by feeding both L-lysine and α-ketoglutarate, with only 5-aminovalerate able to be produced from glucose without supplementation. This appeared to use the same or similar heterologous genes from P. putida as in the previous paper, only expressed together as an artificial operon on one plasmid instead of in two operons on two plasmids. The strain additionally contained a dapA promoter replacement to allow constitutive expression of lysine biosynthesis, and deletion of speE, speG, patA, and puuPA (which would prevent production of spermidine, acetylspermidine, putrescine degradation, and putrescine import, although likely not for any targeted purpose here; use of this background strain for production of other compounds).

Overproduction of adipic acid, as well as other diacids that can be readily converted chemically into adipic acid, has been more heavily pursued due to the use of adipic acid in existing commercial polyamides. A wide variety of routes have been explored, with the first reported direct route in E. coli being a proof-of-concept demonstration, with a maximum titer of 639 μg/mL adipic acid (Yu et al., 2014). In the best-performing strain, acetyl-CoA and succinyl-CoA were condensed by a reversible 3-oxoadipyl-CoA thiolase (PaaJ from E. coli), 3-oxoadipyl-CoA was reduced to 3-hydroxyadipyl-CoA by a 3-hydroxyacyl-CoA dehydrogenase (PaaH1 from Ralstonia eutropha), 3-hydroxyadipyl-CoA was dehydrated to 2,3-dehydroadipyl-CoA by a putative enoyl-CoA hydratase (h16_AA307 gene product from Ralstonia eutropha H16), 2,3-dehydroadipyl-CoA was reduced to adipyl-CoA by a trans-enoyl-CoA reductase (Ter from Euglena gracilis), adipyl-CoA was converted to adipyl-phosphate by a phosphate butyryltransferase (Ptb from Clostridium acetobutylicum), and adipyl-phosphate was finally dephosphorylated to adipic acid using a butyryl kinase (Buk1 from Clostridium acetobutylicum) (see FIG. 1 of Yu et al., 2014, hereby incorporated by reference). The genes encoding these enzymes were heterologously expressed on two different plasmids, and additional modifications were made to the genome to improve the succinyl-CoA supply using modifications that were previously employed for succinic acid production via succinyl-CoA (Liu et al., Process Biochem. 47:1532, 2012). These were deletions of ptsG, poxB, pta, sdhA, and iciR.

Very recently, production of 2.5 g/L adipic acid in bioreactors, as well as smaller quantities of suberic acid (C₈) and sebacic acid (C₁₀), or pimelic acid (C₇) alone, was demonstrated in E. coli from glycerol using a relatively similar modular pathway (Cheong et al., 2016). It was composed of a thiolase capable of condensing a primer and extender unit (e.g. succinyl-CoA and acetyl-CoA for adipic acid), plus a hydroxyacyl-CoA hydrogenase, an enoyl-CoA hydratase, an enoyl-CoA reductase to generate the fully reduced product (for adipic acid, this would be adipyl-CoA) (see FIG. 1a of Cheong et al., 2016, hereby incorporated by reference). The major difference from previous work was the use of an acyl-CoA thioesterase to liberate the final diacid from CoA. For production of adipic acid production, a CoA transferase (Cat1 from Clostridium kluyveri) was expressed for activation of succinic acid to succinyl-CoA, with native sucD encoding a subunit of native E. coli succinyl-CoA synthetase deleted. The thiolase was E. coli PaaJ, the hydroxyacyl-CoA hydrogenase was E. coli PaaH, the enoyl-CoA hydratase was E. coli PaaF, the enoyl-CoA reductase was Ter from Treponema denticola, and the acyl-CoA thioesterase was the dicarboxylic acyl-CoA thioesterase Acot8 from Mus musculus. Additionally, fermentative pathways leading to production of acetate (pta and poxB), lactate (IdhA), and ethanol (adhE) were deleted from the background strain. To produce approximately 25 mg/L pimelic acid, glutaric acid was fed to generate glutaryl-CoA as a primer unit via the action of Cat1 in a background strain deficient in ldhA, poxB, pta, adhE, and the gene encoding fumarate reductase, frdA. To generate a mixture containing predominantly adipic acid at 95 mg/L, but also 34 mg/L suberic acid and 13 mg/L sebacic acid, an alternative thiolase (DcaF), hydroxyacyl-CoA dehydrogenase (DcaH), and enoyl-CoA hydratase (DcaE) from Acinetobacter sp. ADP1, with other enzymes the same as for producing adipic acid. In this case, the background strain was the same as that used for pimelic acid, with additional deletions in a number of other native E. coli acyl-CoA thioesterases (yciA, ybgC, ydiI, tesA, fadM, and tesB).

An alternative native adipate production pathway (reverse adipate degradation) has been reported in the bacterium Thermobifida fusca B6, where succinyl-CoA and acetyl-CoA are condensed by a p-ketothioase (EC 2.3.1.174) to form 3-oxoadipyl-CoA, followed by a series of reactions that are the same as those shown in FIG. 1 of Yu et al., 2014, to form adipyl-CoA. Adipyl-CoA is subsequently converted to adipic acid by a succinyl-CoA synthetase (Tfu_2577, Tfu_2576). A titer of over 2 g/L of adipic acid was obtained by fermentation of T. fusca B6 on glucose and milled corncob (Deng and Mao, 2015). The pathway has not yet been heterologously expressed in other organisms.

Adipic acid production from S. cerevisiae has been described by Verdezyne (e.g. WO 2011/003034 A2), however the starting substrates are fatty acids and the pathway for adipic acid production is therefore very different. The engineered microorganisms described have genetic modifications that add or increase the 6-oxohexanoic acid dehydrogenase, omega oxo fatty acid dehydrogenase, 6-hydroxyhexanoic acid dehydrogenase, omega hydroxyl fatty acid dehydrogenase, hexanoate synthase, monooxygenase, monooxygenase reductase, fatty alcohol oxidase, acyl-CoA ligase, acyl-CoA oxidase, enoyl-CoA hydratase, 3-L-hydroxyacyl-CoA dehydrogenase, and/or acetyl-CoA C-acetyltransferase activities. These modifications suggest a pathway where a fatty acid is broken down into multiple shorter chain diacids, derivatives are condensed with acetyl-CoA to increase the chain length where necessary, and C₆ diacid products are reduced to adipic acid following some similar steps to those shown in FIG. 1a of Cheong et al., supra).

In addition to directly producing the final diacid products, other groups have developed alternative pathways to cis,cis-muconic acid, which can be chemically or enzymatically reduced to adipic acid, or glucaric acid, which can be produced in few steps from glucose and can be chemically reduced to adipic acid. Basic pathway schematics are shown in FIG. 4 of Polen et al. (2013) for muconic acid, and FIG. 5 of Polen et al. (2013) for glucaric acid. FIGS. 4 and 5 of Polen et al. (2013) are hereby incorporated by reference.

Muconic acid has been produced at 141 mg/L from glucose in S. cerevisiae (Curran et al., 2013) in a strain possessing a deletion in ARO3 (a 3-deoxy-D-arabino-heptulosonate-7-phosphate synthase isoform), expression of a feedback resistant version of ARO4 (another 3-deoxy-D-arabino-heptulosonate-7-phosphate synthase isoform; the modifications included both a feedback resistant coding mutation and a constitutive promoter replacement), and a deletion in ZWF1 (glucose-6-phosphate dehydrogenase). The heterologous pathway was expressed on 3 plasmids containing codon-optimized DHS from Podospora anserina, catechol 1,2-dioxygenase from Candida albicans, overexpressed TKL1 (transketolase) from S. cerevisiae, and protocatechuate decarboxylase from Enterobacter cloacae. To obtain higher expression of protocatechuate decarboxylase, another copy of the gene was also integrated onto the chromosome. The combination of non-pathway mutations (deletion of ZWF1 and ARO3; expression of feedback-resistant ARO4; overexpression of TKL1) served to relieve feedback inhibition of the shikimate pathway that is ordinarily employed for aromatic amino acid biosynthesis, and to direct flux into the pentose phosphate pathway via transketolase instead of glucose-6-phosphate dehydrogenase, increasing the supply of the erythrose-4-phosphate precursor.

Muconic acid has additionally been produced (170 mg/L of muconic acid) from glucose in E. coli possessing deletions in ptsH, ptsI, crr, and pykF and overexpressing ubiC, a feedback resistant version of aroF, aroE, and aroL (Sengupta et al., 2015). These mutations increase the supply of the precursors erythrose-4-phosphate and phosphoenolpyruvate. The heterologous pathway was composed of pobA from Pseudomonas putida KT2440, aroY from Klebsiella pneumoniae, and catA from Acinetobacter sp. strain ADP1. E. coli co-cultures consisting of strains engineered to overproduce DHS, and to convert DHS to muconic acid, have additionally been engineered to produce muconic acid from glycerol at a final titer of 2 g/L (Zhang et al., Microb. Cell Factories 14:134, 2015).

### LIST OF REFERENCES

Adkins et al. Engineering Escherichia coli for renewable production of the 5-carbon polyamide building-blocks 5-aminovalerate and glutarate. Biotechnol. Bioeng. 110:1726-34 (2013).
Park et al. Metabolic engineering of Escherichia coli for the production of 5-aminovalerate and glutarate as C5 platform chemicals. Metab. Eng. 16:42-7 (2013).
Yu et al. Direct biosynthesis of adipic acid from a synthetic pathway in recombinant Escherichia coli. Biotechnol. Bioeng. 111:2580-6 (2014).
Cheong et al. Energy- and carbon-efficient synthesis of functionalized small molecules in bacteria using non-decarboxylative Claisen condensation reactions. Nat Biotech 34:556-61 (2016).
Deng and Mao. Production of adipic acid by the native-occurring pathway in Thermobifida fusca B6. J. Appl. Microbiol., 119:1057-63 (2015).
Curran et al. Metabolic engineering of muconic acid production in Saccharomyces cerevisiae. Metab. Eng. 15:55-66 (2013).
Polen et al. Toward biotechnological production of adipic acid and precursors from biorenewables. J. Biotechnol, 167:75-84 (2013).
Sengupta et al. Metabolic engineering of a novel muconic acid biosynthesis pathway via 4-hydroxybenzoic acid in Escherichia coli. Appl. Environ. Microbiol. 81:8037-43 (2015).
Zhang et al. Engineering E. coli-E. coli cocultures for production of muconic acid from glycerol. Microb. Cell Factories 14:134-(2015).
Van Dien S. From the first drop to the first truckload: commercialization of microbial processes for renewable chemicals. Gurr. Opin. Biotechno. 24:1-8 (2013).
Zingaro K A, et al. Dissecting the assays to assess microbial tolerance to toxic chemicals in bioprocessing. Trends Biotechnol. 31:643-653 (2013).
Haft R J F, et al. Correcting direct effects of ethanol on translation and transcription machinery confers ethanol tolerance in bacteria. Proc. Natl. Acad. Sci. USA 111:E2576-E2585 (2014).
Sandberg T E, et al. Evolution of Escherichia coli to 42° C. and subsequent genetic engineering reveals adaptive mechanisms and novel mutations. Mol. Biol. Evol. 31:2647-2662 (2014).
Lennen R M, Herrgard M J. Combinatorial strategies for improving multiple-stress resistance in industrially relevant Escherichia coli strains. Appl. Environ. Microbiol. 80:6223-6242 (2014).
Tenaillon O, Rodriguez-Verdugo A, Gaut R L, McDonald P, Bennett A F, Long A D, Gaut B S. The molecular diversity of adaptive convergence. Science 335:457-461 (2012).
Minty J J, et al. Evolution combined with genomic study elucidates genetic bases of isobutanol tolerance in Escherichia coli. Microbial Cell Factories 10:18 (2011).
Dragosits M, Mozhayskiy V, Quinones-Soto S, Park J, Tagkopoulos I. Evolutionary potential, cross-stress behavior and the genetic basis of acquired stress resistance in Escherichia coli. Mol. Syst. Biol. 9:643 (2013a).
Dragosits M, Mattanovich D. Adaptive laboratory evolution—principles and applications for biotechnology, Microbial Gell Factories 12:64 (2013b).
Winkler J D, et al. Evolved osmotolerant Escherichia coli mutants frequently exhibit defective Nacetylglucosamine catabolism and point mutations in the cell shape-regulating protein MreB. Appl. Environ. Microbiol. 80:3729-3740 (2014).
Wu X, Altman R, Eiteman M A, Altman E. Adaptation of Escherichia coli to elevated sodium concentrations increases cation tolerance and enables greater lactic acid production. Appl. Environ. Microbiol. 80:2880-2888 (2014).
LaCroix R A, et al. Use of adaptive laboratory evolution to discover key mutations enabling rapid growth of Escherichia coli K-12 MG1655 on glucose minimal medium. Appl. Environ. Microbiol. 81:17-30 (2015).
Jensen S I, Lennen R M, Herrgard M J, Nielsen A T. Seven deletions in seven days: Fast generation of Escherichia coli strains tolerant to acetate and osmotic stress. Sci. Rep., accepted.
Doukyu N, Ishikawa K, Watanabe R, Ogino H. Improvement in organic solvent tolerance by double disruption of proV and marR genes in Escherichia coli. J. Appl. Microbiol. 112:464-474 (2012).
Baba T et al., Construction of Escherichia coli K-12 in-frame, single-gene knockout mutants: the Keio collection. Mol. Syst. Biol. 2:2006.0008 (2006).
Jensen et al. The Escherichia coli K-12 “wild types” W3110 and MG1655 have an rph frameshift mutation that leads to pyrimidine starvation due to low pyrE expression levels. J. Bacteriol. 175:3401-3407 (1993).
Conrad et al. Whole-genome resequencing of Escherichia coli K-12 MG1655 undergoing short-term laboratory evolution in lactate minimal media reveals flexible selection of adaptive mutations. Genome Biol. 10:R118 (2009).
Datsenko K A, Wanner B L. One-step inactivation of chromosomal genes in Escherichia coli K-12 using PCR products. Proc. Natl. Acad. Sci. USA 97:6640-6645 (2000).
Thomason L C, Constantino N, Court D L. E. coli genome manipulation by P1 transduction. Curr. Protoc. Molec. Biol. 1.17 (2007a).
Thomason L, et al. Recombineering: Genetic engineering in bacteria using homologous recombination. Curr. Protoc. Molec. Biol. 1.16 (2007b).
Wang H H, Isaacs F J, Carr P A, Sun Z Z, Xu G, Forest C R, Church G M. Programming cells by multiplex genome engineering and accelerated evolution. Nature 460:894-898 (2009).
Byrne et al. Comparative multi-goal tradeoffs in systems engineering of microbial metabolism. BMC Systems Biology, vol. 6, no. 1, 2012, page 127.
Lacroix et al. Use of Adaptive Laboratory Evolution To Discover Key Mutations Enabling Rapid Growth of Escherichia coli K-12 MG1655 on Glucose Minimal Medium. Applied and Environmental Microbiology, vol. 81, no. 1, 2014, pages 17-30.
Lee et al. Characteristics of acid tolerance of adipic acid-resistant mutant strain, Leuconostoc mesenteroides. Korean Journal of Applied Microbiology and Bioengineering, vol. 28, no. 2, April 2000, pages 63-70.
Adkins et al. Engineering Escherichia coli for renewable production of the 5-carbon polyamide building-blocks 5-aminovalerate and glutarate. Biotechnology and Bioengineering, vol. 110, no. 6, 2013, pages 1726-1734.
WO 2011/003034 A2 (Verdezyne)
US 2012/0282661 A1 (Genomatica Inc.)
WO 2009/086075 A1 and US 2009/0162911 A1 (E I Du Pont De Nemours and Company)
US 2003/0068611 A1 (E I DuPont De Nemours and Company)
WO 2009/143455 A2 (Microbia Inc.)
WO 2011/154503 A1 (Evonik Degussa GmbH)
US 2014/0135526 A1 (OPX Biotechnologies Inc.)
WO 2014/029592 A1 (Evonik Degussa GmbH)
EP 2 032 711 A1 (TNO)
EP 2 580 315 A2 (Cobalt Technologies Inc.)
WO 01/05959 A1 (Ajinomoto K K)
WO 2004/033421 A2 (Genencor Int)
WO 2016/162442 A1 (Metabolic Explorer SA)

1. A bacterial cell comprising a biosynthetic pathway for producing an aliphatic dicarboxylic acid and at least one genetic modification which reduces expression of an endogenous gene selected from the group consisting of kgtP, ybjL, proV, proW, proX, proQ, cspE, rfaE, yfbP, yfjM, pstS, pstA, pstB, pstC, rph, rpoS, sspA, tdk, uvrB, ycjG, and yeaR, or a combination of any thereof.

2. The bacterial cell of claim 1, comprising a genetic modification which reduces the expression of kgtP.

3. The bacterial cell of claim 1, comprising at least one genetic modification which reduces the expression of ybjL, proV, proW, proX, sspA or a combination of any thereof.

4. The bacterial cell of claim 1, comprising genetic modifications which reduce the expression of
(a) kgtP and at least one of proV, proW and proX;
(b) kgtP and ybjL;
(c) kgtP, ybjL, and at least one of proV, proW and proX;
(d) kgtP, ybjL, and at least one of nagA and nagC;
(e) kgtP, ybjL, at least one of nagA and nagC, and at least one of proV, proW and proX;
(f) kgtP and sspA; or
(g) kgtP, tdk and pstS.

5. The bacterial cell of claim 1, wherein the genetic modification comprises a knock-down or knock-out of the endogenous gene or genes.

6. The bacterial cell of claim 1, wherein the genetic modification provides for an increased growth rate, a reduced lag time, or both, of the cell in the presence of at least one of glutaric acid and adipic acid as compared to the bacterial cell without the genetic modification.

7. The bacterial cell of claim 1, genetically modified from a parent bacterial cell so as to comprise
(a) a mutant SpoT, comprising at least one mutation in the threonyl-tRNA synthetase GTPase and SpoT (TGS) domain corresponding to amino acid residues 1388 to T447 and/or the linker segment between the TGS and the aspartokinase, chorismate mutase and TyrA (ACT) domain corresponding to amino acid residues A448 to T621, optionally in one or more amino acid residues selected from A451, R236, V422, W457, N454, D580, M247, T442, S434, N601, 1602 and R603;
(b) a mutant PolB, comprising a mutation in amino acid residue R477;
(c) a mutant RpoC, comprising a mutation in at least one of the amino acid residues corresponding to H419 and P64;
(d) a mutant RpoB, comprising a mutation in an amino acid residue corresponding to K203;
(e) a mutant Rnt, comprising a mutation in at least one of the amino acid residues corresponding to Q179, A27, F194 and A180;
(f) a mutant SapC, comprising a mutation in the amino acid residue corresponding to G79;
(g) increased expression of PyrE as compared to the parent bacterial cell; or
(h) a combination of any two or more of (a) to (g),
wherein the genetic modification provides for an increased growth rate, a reduced lag time, or both, in the presence of at least one of glutaric acid and adipic acid as compared to the parent bacterial cell.

8. The bacterial cell of claim 7, comprising
(a) at least one mutant protein selected from the group consisting of SpoT-V422A, SpoT-A451D, SpoT-A451V, SpoT-W457C, SpoT-N454H, SpoT-D580Y, SpoT-R236L, SpoT-R236S, SpoT-M247K, SpoT-NIR(601-603)S, SpoT-T442I, SpoT-S434L, PolB-R477G, RpoC-H419P, RpoC-P64L, RpoB-K203T, Rnt-Q179P, Rnt-A27T, Rnt-F194L, Rnt-A180T, SapC-G79W; and/or
(b) a mutation in rph or the pyrE/rph intergenic region which increases the expression of PyrE.

9. The bacterial cell of claim 1, comprising a recombinant biosynthetic pathway for producing at least one of glutaric acid, adipic acid, succinic acid, muconic acid, fumaric acid, itaconic acid, malic acid, malonic acid, maleic acid, glucaric acid, pimelic acid, suberic acid, sebacic acid, 2,5-furandicarboxylic acid, terephthalic acid, or azelaic acid, mesaconic acid, citraconic acid, tartaric acid, tartronic acid, diaminopimelic acid and glutaconic acid.

10. A process for preparing a bacterial cell of claim 5, the process comprising genetically modifying an E. coli cell to
(a) introduce a recombinant biosynthetic pathway for producing the aliphatic dicarboxylic acid, and
(b) knock-down or knock-out of the endogenous gene or genes according to claim 5, and/or
wherein steps (a) and (b) are performed in any order.

11. A process for improving the tolerance of a bacterial cell to an aliphatic dicarboxylic acid comprising genetically modifying the bacterial cell to
(a) knock-down or knock-out of the endogenous gene or genes according to claim 5.

12. The bacterial cell of claim 1, wherein the bacterial cell is of the Escherichia, Lactobacillus, Lactococcus, Bacillus, Pseudomonas, Corynebacterium, Deinococcus or Ralstonia species, such as of the Escherichia coli species.

13. A method for producing an aliphatic dicarboxylic acid, comprising culturing the bacterial cell of claim 1, in the presence of a carbon source, and, optionally, isolating the aliphatic dicarboxylic acid.

14. A composition comprising glutaric acid or adipic acid at a concentration of at least 5 g/L and a plurality of bacterial cells according to claim 1

15. The bacterial cell of claim 1, wherein the biosynthetic pathway for producing an aliphatic dicarboxylic acid comprises
(a) a lysine monooxygenase, a 5-aminovaleramidase, a 5-aminovalerate transaminase, and a glutaraldehyde semialdehyde dehydrogenase;
(b) a reversible 3-oxoadipyl-CoA thiolase, a 3-hydroxyacyl-CoA dehydrogenase, an enoyl-CoA hydratase, an enoyl-CoA reductase, and either a terminal carboxyacyl-CoA thioesterase, or a terminal carboxyacyl-CoA phosphotransferase and a reversible alkyl-1,n-dicarboxylate kinase, where n is the carbon chain length of the product; and, optionally a malonyl-CoA or glutaryl-CoA transferase; or
(c) a 2-dehydro-3-deoxy-D-arabinoheptonate-7-phosphate synthase, a 3-dehydroquinate synthase, a 3-dehydroxyquinate dehydratase, a dehydroshikimic acid dehydratase, a protocatechuate decarboxylase, and a catechol 1,2-dioxygenase.

16. The process of claim 10 comprising genetically modifying the E. coli cell to
(c) express a mutant of at least one of SpoT, PolB, RpoC, RpoB, Rnt and SapC and/or increase expression of PyrE,
wherein steps (a), (b) and (c) are performed in any order.

17. The process of claim 11 comprising genetically modifying the bacterial cell to
(b) express a mutant of at least one of SpoT, PolB, RpoC, RpoB, Rnt and SapC and/or increase expression of PyrE according to claim 7;
Wherein steps (a) and (b) are performed in any order.
